(19) United States
(12) Patent Application Publication
(10) Pub. No.: US 2021/0393802 A1
(43) Pub. Date: Dec. 23, 2021

(54) GENE THERAPY FOR NEURONAL CEROID LIPOFUSCINOSES

(71) Applicant: REGENXBIO INC., Rockville, MD (US)

(72) Inventors: Bhargavi Kondragunta, Clarksburg, MD (US); Subha Karumuthil Melethil, Germantown, MD (US); Matthieu Pierre Guibert, Germantown, MD (US); Andrew Christopher Mercer, Poolesville, MD (US); Micheal James Carrell, Mt. Airy, MD (US); Robert Thomas Stadelman, New Market, MD (US); Franz Michael Gerner, Myersville, MD (US); Nicholas Alexander Piers Sascha Buss, Rockville, MD (US); Jared Bee, Gaithersburg, MD (US); Tristan James Marshall, Potomac, MD (US); Roberto DePaz, Bethesda, MD (US)

(21) Appl. No.: 17/292,750

(22) PCT Filed: Nov. 13, 2019
(86) PCT No.: PCT/US2019/061206

§ 371 Date: May 11, 2021

Related U.S. Application Data
(60) Provisional application No. 62/767,410, filed on Nov. 14, 2018.
(60) Provisional application No. 62/924,060, filed on Oct. 21, 2019.

(51) Int. Cl. A61K 48/00; A61P 25/00

(52) U.S. Cl. CPC A61K 48/0008; A61P 25/00; A61K 48/0075

(57) ABSTRACT

Provided herein are methods and compositions for treatment of Batten disease. Such compositions include a recombinant adeno-associated virus (rAAV), said rAAV comprising an AAV capsid, and a vector genome packaged therein, said vector genome comprising (a) an AAV 5′ inverted terminal repeat (ITR) sequence; (b) a promoter; (c) a CLN2 coding sequence encoding a human TPP1; (d) an AAV 3′ ITR. Also provided herein are methods of treating Batten disease comprising administering to a subject in need thereof the rAAV described herein via more than one route. Also provide herein are pharmaceutical compositions comprising the rAAV described herein and related methods of treating Batten disease.

This application claims the benefit of U.S. Provisional Application Ser. No. 62/767,410, filed on Nov. 14, 2018 and U.S. Provisional Application Ser. No. 62/924,060, filed on Oct. 21, 2019, both of which are incorporated herein by reference in their entirety.

### REFERENCE TO SEQUENCE LISTING SUBMITTED ELECTRONICALLY

This application incorporates by reference a Sequence Listing submitted with this application as text file entitled “12656-125-228 SEQ LISTING” created on Nov. 3, 2019 and having a size of 36,393 bytes.

### BACKGROUND OF THE INVENTION

The neuronal ceroid lipofuscinoses (NCLs) are a group of rare and inherited neurodegenerative disorders. They are considered the most common of the neurogenetic storage diseases, with the accumulation of autofluorescent lipopigments resembling ceroid and lipofuscin seen in patients. NCLs are associated with variable, yet progressive, symptoms, including abnormally increased muscle tone or spasm, blindness or vision problems, dementia, lack of muscle coordination, intellectual disability, movement disorder, seizures and unsteady walk. The frequency of this disease is approximately 1 per 12,500 individuals. There are three main types of NCL: adult (Kufs or Parry disease); juvenile and late infantile (Jansky-Bielschowsky disease). The neuronal ceroid lipofuscinoses (NCLs) originally were defined by their age of onset and clinical symptoms (as noted herein). However, they have since been reclassified on the basis of newer molecular findings, which have provided evidence of far more overlap for the different genetic variants than had previously been suggested by the clinical phenotypes.

At least twenty genes have been identified in association with NCL. NCL patients with CLN2 mutations are deficient in a pepstatin-insensitive lysosomal peptidase called tripeptidyl peptidase 1 (TTP1). TTP1 removes tripeptides from the N-terminal of polypeptides. Mutations have been reported in all 13 exons of the CLN2 gene. Some mutations result in a more protracted course. Although onset is usually in late infancy, later onset has been described. More than 58 mutations have been described in CLN2.

CLN2 disease, a form of Batten disease, is a rare lysosomal storage disorder (LSD) with an estimated incidence of 0.07-0.51 per 100,000 live births (Augestad et al., 2006; Claussen et al., 1992; Mole et al., 2013; National Batten Disease Registry; Poupetova et al., 2010; Santorelli et al., 2013; Teixeira et al., 2003). CLN2 disease is a fatal autosomal recessive neurodegenerative LSD caused by mutations in the CLN2 gene, located on chromosome 11q15 and encoding for the soluble lysosomal enzyme tripeptidyl-peptidase-1 (TPP1). Mutations in the CLN2 gene, and subsequent deficiency in TPP1 enzymatic activity, result in lysosomal accumulation of storage material and neurodegeneration of the brain and retina (Liu et al., 1998; Wlodawer et al., 2003). CLN2 disease is characterized by early onset at 2-4 years of age with initial features usually including recurrent seizures (epilepsy) and difficulty coordinating movements (ataxia). The disease also results in the loss of previously acquired skills (developmental regression). Epilepsy is often refractory to medical therapy, and the general decay of psychomotor functions is rapid and uniform between the third and fifth birthday (Schulz et al., 2013) before premature death by mid-childhood (Nickel M et al., 2016; Worgall et al., 2007).

Enzyme replacement therapy (ERT) with recombinant TPP1 (Brineura® cerliponase alfa, BioMarin Pharmaceuticals) was recently approved in the United States (US) and European Union (EU) for the treatment of CLN2 disease and is administered as a biweekly infusion into the lateral ventricles via a permanently implanted device. The clinical benefit of Brineura® was designated to be limited to stabilization of motor function by the FDA, while the European Medicines Agency (EMA) determined that there was a positive impact on language skills as well (Brineura®, FDA Summary Basis of Approval; Brineura® European Public Assessment Report [EPAR]; Schulz et al., 2016). Brineura® requires specialized expertise for the implantation of a port directly into the brain and must be administered during a 4-hour infusion every two weeks in a healthcare setting by a trained professional knowledgeable in intracerebroventricular (ICV) administration. Repeat infusions are necessary in part due to the short CSF and lysosomal half-lives of Brineura® which are estimated to be 7 hours and 11.5 days, respectively (Brineura®, EPAR). Thus, there remains a significant unmet need for new therapies that can provide durable and long-term TPP1 enzymatic activity in the central nervous system (CNS) of patients with CLN2 disease, without the high patient burden and morbidities associated with repeat administration of ERT. Therefore, compositions useful for delivering and expressing TPP1 in subjects in need for treating CLN2 disease are needed. A one-time administration of recombinant adeno-associated virus (rAAV) expressing canine TPP1 (rAAV2.caTPP1) was shown to result in high expression of TPP1 predominantly in ependymal cells and secretion of the enzyme into the cerebrospinal fluid leading to clinical benefit. See Katz et al, Sci Transl Med. 2015 Nov. 11; 7(313): 313ra180; and KATZ, et al, Gene therapy 2017 Feb. 24(4): 215-223, which are incorporated herein by reference. However AAV2 does not penetrate the brain parenchyma and does not target neurons, thus limiting the expected benefits compared to what can be achieved with novel neurotropic AAVs.

### SUMMARY OF THE INVENTION

Provided herein is an aqueous suspension suitable for administration to subject having Batten disease is provided. In one embodiment, the suspension includes an aqueous suspending liquid and about 7.5×10¹² GC (7.5×10⁹ GC/gram of brain) to about 2.7×10¹⁵ GC (2.1×10¹² GC/gram of brain) or viral particles of a recombinant adeno-associated virus (rAAV) useful as a therapeutic for Batten, said rAAV having an AAV capsid, and having packaged therein a vector genome comprising: (a) an AAV 5′ inverted terminal repeat (ITR) sequence; (b) a promoter; (c) a CLN2 coding sequence encoding a human TPP1; and (d) an AAV 3′ ITR.

Also provided herein is a method of treating CLN2 Batten disease in a subject comprising administering to a subject in need thereof a recombinant adeno-associated virus (rAAV) via a first route and a second route, and said first route and said second route are into the central nervous system (CNS), and said first route is into the brain region and said second route is into the spinal cord region, and said recombinant adeno-associated virus (rAAV) comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR.

In certain embodiments, said first route is intracerebroventricular (ICV) or intracisternal (IC). In certain embodiments, said second route is intrathecal-lumbar (IT-L). In certain embodiments, said method further comprises administering to said subject said rAAV via a third route, wherein said third route is selected from the group consisting of intracerebroventricular (ICV), intracisternal (IC), intrathecal-lumbar, intracranial, intravenous, intravascular, intraarterial, intramuscular, intraocular, subcutaneous, and intradermal.

In certain embodiments, the method of treating CLN2 Batten disease in a subject comprises co-administering to a subject in need thereof said rAAV via intracerebroventricular (ICV) and intrathecal-lumbar (IT-L) routes. In certain embodiments, the method of treating CLN2 Batten disease in a subject comprises co-administering to a subject in need thereof said rAAV via intracisternal (IC) and intrathecal-lumbar (IT-L) routes. In certain embodiments, the method of treating CLN2 Batten disease in a subject comprises co-administering to a subject in need thereof said rAAV via intracerebroventricular (ICV), intrathecal-lumbar (IT-L), and intravenous routes. In certain embodiments, the method of treating CLN2 Batten disease in a subject comprises co-administering to a subject in need thereof said rAAV via intracisternal (IC), intrathecal-lumbar (IT-L), and intravenous routes.

In another aspect, provided is a method of treating CLN2 Batten disease in a subject comprising administering to a subject in need thereof a recombinant adeno-associated virus (rAAV) via a first route and a second route, and said first route is into the central nervous system (CNS), and said second route delivers the rAAV outside of the CNS, and said recombinant adeno-associated virus (rAAV) comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, said first route is intrathecal-lumbar (IT-L), intracerebroventricular (ICV) or intracisternal (IC). In certain embodiments, said second route is selected from the group consisting of intravenous, intravascular, intraarterial, intramuscular, intraocular, subcutaneous, and intradermal. In a specific embodiment, said second route is intravenous.

In another aspect, provided is a method of treating CLN2 Batten disease in a subject comprising administering to a subject in need thereof a recombinant adeno-associated virus (rAAV) via a first route and a second route, and said first route is into the central nervous system (CNS), and said second route delivers the rAAV to the liver, and said recombinant adeno-associated virus (rAAV) comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, said first route is intrathecal-lumbar (IT-L), intracerebroventricular (ICV) or intracisternal (IC). In certain embodiments, said second route is selected from the group consisting of intravenous, intravascular, intraarterial, intramuscular, intraocular, subcutaneous, and intradermal. In a specific embodiment, said second route is intravenous.

In certain embodiments, the method of treating CLN2 Batten disease in a subject comprises co-administering to a subject in need thereof said rAAV via intrathecal and intravenous routes. In certain embodiments, the method of treating CLN2 Batten disease in a subject comprises co-administering to a subject in need thereof said rAAV via intrathecal-lumbar (IT-L) and intravenous routes. In certain embodiments, the method of treating CLN2 Batten disease in a subject comprises co-administering to a subject in need thereof said rAAV via intracerebroventricular (ICV) and intravenous routes. In certain embodiments, the method of treating CLN2 Batten disease in a subject comprises co-administering to a subject in need thereof said rAAV via intracisternal (IC) and intravenous routes.

In certain embodiments, methods of treating CLN2 Batten disease provided herein may comprise administering said rAAV via said first route simultaneously with administering said rAAV via said second route.

In certain embodiments, methods of treating CLN2 Batten disease provided herein may comprise administering said rAAV via said first route prior to administering said rAAV via said second route. In certain embodiments, methods of treating CLN2 Batten disease provided herein may comprise administering said rAAV via said first route after administering said rAAV via said second route.

In certain embodiments, the methods of treating CLN2 Batten disease provided herein may result in an increased TPP1 activity in the spinal cord of said subject. In certain embodiments, the methods of treating CLN2 Batten disease provided herein may result in an increased hepatic TPP1 activity of said subject. In certain embodiments, the methods of treating CLN2 Batten disease provided herein may result in an increased serum TPP1 activity of said subject. In certain embodiments, the methods of treating CLN2 Batten disease provided herein may result in a reduced microglial activity in the cortex of said subject. In certain embodiments, the methods of treating CLN2 Batten disease provided herein may result in an increase TPP1 activity in the brain of said subject.

In certain embodiments, said rAAV is administered in a therapeutically effective amount.

In certain embodiments, said subject is human.

In certain embodiments, the coding sequence of (c) is a codon optimized human CLN2, which is at least 70% identical to the native human coding sequence of SEQ ID NO: 2. In certain embodiments, the coding sequence of (c) is SEQ ID NO: 3.

In certain embodiments, the rAAV capsid is an AAV9 or a variant thereof.

In certain embodiments, the promoter is a chicken beta actin (CBA) promoter. In certain embodiments, the promoter is a hybrid promoter comprising a CBA promoter sequence and cytomegalovirus enhancer elements.

In certain embodiments, the AAV 5′ ITR and/or AAV3′ ITR is from AAV2.

In certain embodiments, the vector genome further comprises a polyA. In certain embodiments, the polyA is a synthetic polyA or from bovine growth hormone (bGH), human growth hormone (hGH), SV40, rabbit β-globin (RGB), or modified RGB (mRGB).

In certain embodiments, the vector genome further comprises an intron. In certain embodiments, the intron is from CBA, human beta globin, IVS2, SV40, bGH, alpha-globulin, beta-globulin, collagen, ovalbumin, or p53.

In certain embodiments, the vector genome further comprises an enhancer. In certain embodiments, the enhancer is a CMV enhancer, an RSV enhancer, an APB enhancer, ABPS enhancer, an alpha mic/bik enhancer, TTR enhancer, en34, ApoE.

In certain embodiments, the vector genome is about 3 kilobases to about 5.5 kilobases in size. In certain embodiments, the vector genome is about 4 kilobases in size.

In certain embodiments, the rAAV is manufactured using a method comprising growing in suspension culture a suspension cell line that is capable of producing the rAAV. In certain embodiments, said suspension cell line is HEK293 suspension cell line.

Also provided herein are pharmaceutical compositions. In certain embodiments, provided herein is a pharmaceutical composition comprising:

(a) a recombinant adeno-associated virus (rAAV),
(b) sodium chloride,
(c) magnesium chloride,
(d) potassium chloride,
(e) dextrose,
(f) poloxamer 188,
(g) sodium phosphate monobasic, and
(h) sodium phosphate dibasic,
wherein said recombinant adeno-associated virus (rAAV) comprises an AAV capsid and a vector genome packaged therein, and wherein said vector genome comprising: (i) an AAV 5′ inverted terminal repeat (ITR) sequence; (ii) a promoter; (iii) a CLN2 coding sequence encoding a human TPP1; and (iv) an AAV 3′ ITR.

In certain embodiments, the pharmaceutical composition further comprising calcium chloride.

In certain embodiments, said sodium chloride, said magnesium chloride, said potassium chloride, said dextorse, said poloxamer 188, said sodium phosphate monobasic, said sodium phosphate dibasic, and said calcium chloride are each in anhydrous, monohydrate, dihydrate, 3-hydrate, 4-hydrate, 5-hydrate, 6-hydrate, 7-hydrate, 8-hydrate, 9-hydrate, or 10-hydrate form.

In certain embodiments, the pharmaceutical composition comprises

(a) said rAAV,
(b) sodium chloride at a concentration of about 8.77 g/L,
(c) magnesium chloride 6-hydrate, at a concentration of about 0.244 g/L,
(d) potassium chloride at a concentration of about 0.224 g/L,
(e) calcium chloride dihydrate at a concentration of about 0.206 g/L,
(f) dextorse anhydrous at a concentration of about 0.793 g/L,
(g) poloxamer 188 at a concentration of about 0.001% (volume/volume),
(h) sodium phosphate monobasic monohydrate at a concentration of about 0.0278 g/L, and
(i) sodium phosphate dibasic anhydrous at a concentration of about 0.114 g/L.

In certain embodiments, the vector genome concentration (VGC) of the pharmaceutical composition is about 1×10¹¹ GC/mL, about 3×10¹¹ GC/mL, about 6×10¹¹ GC/mL, about 1×10¹² GC/mL, about 3×10¹² GC/mL, about 6×10¹² GC/mL, about 1×10¹³ GC/mL, about 2×10¹³ GC/mL, about 3×10¹³ GC/mL, about 4×10¹³ GC/mL, about 5×10¹³ GC/mL, about 6×10¹³ GC/mL, about 7×10¹³ GC/mL, about 8×10¹³ GC/mL, about 9×10¹³ GC/mL, or about 1×10¹⁴ GC/mL, about 3×10¹⁴ GC/mL, about 6×10¹⁴ GC/mL, or about 1×10¹⁵ GC/mL.

In certain embodiments, the pH of the pharmaceutical composition is in a range from about 6.0 to about 9.0. In certain embodiments, the pH of the pharmaceutical composition is about 7.4.

In certain embodiments, said rAAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant rAAV in a reference pharmaceutical composition. In certain embodiments, the stability of said rAAV in the pharmaceutical composition is determined by

(a) the infectivity of rAAV,
(b) the levels of aggregation of rAAV, or
(c) the levels of free DNA released by the rAAV.

In certain embodiments, the pharmaceutical composition is a liquid composition. In certain embodiments, the pharmaceutical composition is a frozen composition. In certain embodiments, the pharmaceutical composition is a lyophilized composition or a reconstituted lyophilized composition.

In certain embodiments, the pharmaceutical composition has a property that is suitable for intracerebroventricular (ICV), intracisternal (IC), intrathecal-lumbar, intracranial, intravenous, intravascular, intraarterial, intramuscular, intraocular, intramuscular, subcutaneous, or intradermal administration.

In certain embodiments, the coding sequence of (iii) of the rAAV in the pharmaceutical composition is a codon optimized human CLN2, which is at least 70% identical to the native human coding sequence of SEQ ID NO: 2. In certain embodiments, the coding sequence of (iii) of the rAAV in the pharmaceutical composition is SEQ ID NO: 3.

In certain embodiments, the rAAV capsid of the rAAV in the pharmaceutical composition is an AAV9 or a variant thereof.

In certain embodiments, the promoter of the rAAV in the pharmaceutical composition is a chicken beta actin (CBA) promoter. In certain embodiments, the promoter of the rAAV in the pharmaceutical composition is a hybrid promoter comprising a CBA promoter sequence and cytomegalovirus enhancer elements.

In certain embodiments, the AAV 5′ ITR and/or AAV3′ ITR of the rAAV in the pharmaceutical composition is from AAV2.

In certain embodiments, the vector genome of the rAAV in the pharmaceutical composition further comprises a polyA. In certain embodiments, the polyA is a synthetic polyA or from bovine growth hormone (bGH), human growth hormone (hGH), SV40, rabbit 3-globin (RGB), or modified RGB (mRGB).

In certain embodiments, the vector genome of the rAAV in the pharmaceutical composition further comprises an intron. In certain embodiments, the intron is from CBA, human beta globin, IVS2, SV40, bGH, alpha-globulin, beta-globulin, collagen, ovalbumin, or p53.

In certain embodiments, the vector genome of the rAAV in the pharmaceutical composition further comprises an enhancer. In certain embodiments, the enhancer is a CMV enhancer, an RSV enhancer, an APB enhancer, ABPS enhancer, an alpha mic/bik enhancer, TTR enhancer, en34, ApoE.

In certain embodiments, the vector genome of the rAAV in the pharmaceutical composition is about 3 kilobases to about 5.5 kilobases in size. In certain embodiments, the vector genome of the rAAV in the pharmaceutical composition is about 4 kilobases in size.

In certain embodiments, the rAAV in the pharmaceutical composition is manufactured using a method comprising growing in suspension culture a suspension cell line that is capable of producing the rAAV.

In another aspect, provide herein is a method of treating CLN2 Batten disease in a subject comprising administering to said subject the pharmaceutical composition provided herein. In certain embodiments, said pharmaceutical composition is administered in a therapeutically effective amount. In certain embodiments, said subject is human.

In yet another aspect, provide herein is a kit comprising one or more containers and instructions for use, wherein the one or more containers comprise the pharmaceutical composition provided herein.

Also provided herein are methods of manufacturing a rAAV. In certain embodiments, the method comprises growing in suspension culture a suspension cell line that is capable of producing the rAAV.

Other aspects and embodiments will be readily apparent based on the information described herein.

### Illustrative Embodiments

1. A method of treating CLN2 Batten disease in a subject comprising administering to a subject in need thereof a recombinant adeno-associated virus (rAAV) via a first route and a second route, wherein said first route and said second route are into the central nervous system (CNS), wherein said first route is into the brain region and said second route is into the spinal cord region, and
wherein said recombinant adeno-associated virus (rAAV) comprises an AAV capsid and a vector genome packaged therein, and wherein said vector genome comprising:

(a) an AAV 5′ inverted terminal repeat (ITR) sequence;

(b) a promoter;

(c) a CLN2 coding sequence encoding a human TPP1; and

(d) an AAV 3′ ITR.

2. The method of paragraphs 1, wherein said first route is intracerebroventricular (ICV) or intracisternal (IC).
3. The method according to any one of paragraphs 1 to 2, wherein said second route is intrathecal-lumbar (IT-L).
4. The method according to any one of paragraphs 1 to 3, wherein said method further comprises administering to said subject said rAAV via a third route, wherein said third route is selected from the group consisting of intracerebroventricular (ICV), intracisternal (IC), intrathecal-lumbar, intracranial, intravenous, intravascular, intraarterial, intramuscular, intraocular, subcutaneous, and intradermal.
5. A method of treating CLN2 Batten disease in a subject comprising administering to a subject in need thereof a recombinant adeno-associated virus (rAAV) via a first route and a second route, wherein said first route is into the central nervous system (CNS), wherein said second route delivers the rAAV to the liver, and
wherein said recombinant adeno-associated virus (rAAV) comprises an AAV capsid and a vector genome packaged therein, and wherein said vector genome comprising:

(a) an AAV 5′ inverted terminal repeat (ITR) sequence;

(b) a promoter;

(c) a CLN2 coding sequence encoding a human TPP1; and

(d) an AAV 3′ ITR.

6. The method of paragraphs 5, wherein said first route is intrathecal-lumbar (IT-L), intracerebroventricular (ICV) or intracisternal (IC).
7. The method according to any one of paragraphs 5 to 6, wherein said second route is selected from the group consisting of intravenous, intravascular, intraarterial, intramuscular, intraocular, subcutaneous, and intradermal.
8. The method according to paragraph 7, wherein said second route is intravenous.
9. The method according to any one of paragraphs 1 to 8, wherein said method comprises administering said rAAV via said first route simultaneously with administering said rAAV via said second route.
10. The method according to any one of paragraphs 1 to 8, wherein said method comprises administering said rAAV via said first route prior to administering said rAAV via said second route.
11. The method according to any one of paragraphs 1 to 8, wherein said method comprises administering said rAAV via said first route after administering said rAAV via said second route.
12. The method according to any one of paragraphs 10 to 11, wherein the interval between administration said rAAV via said first route and administering said rAAV via said second route is about 0.5 hour, about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.
13. The method according to any one of paragraphs 1 to 12, wherein said method results in a TPP1 activity in the spinal cord of said subject that is at least 2%, 3%, 5%, 6%, 7%, 8%, 9%, 10%, 12%, 15%, 17%, 20%, 25%, 30%, 35%, 40%, 45%, or 50% higher than a reference TPP1 activity in the spinal cord of a second subject, and wherein the reference TPP1 activity in the spinal cord is measured when said second subject does not receive the treatment using said method, and wherein said second subject is the same or different from said subject.
14. The method according to any one of paragraphs 1 to 13, wherein said method results in a hepatic TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference hepatic TPP1 activity in a second subject, and wherein the reference hepatic TPP1 activity is measured when said second subject does not receive the treatment using said method, and wherein said second subject is the same or different from said subject.
15. The method according to any one of paragraphs 1 to 14, wherein said method results in a serum TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference serum TPP1 activity in a second subject, and wherein the reference serum TPP1 activity is measured when said second subject does not receive the treatment using said method, and wherein said second subject is the same or different from said subject.
16. The method according to any one of paragraphs 1 to 15, wherein said method results in a microglial activity in the cortex of said subject that is at least 2%, 3%, 5%, 6%, 7%, 8%, 9%, 10%, 12%, 15%, 17%, 20%, 25%, 30%, 35%, 40%, 45%, or 50% lower than a reference microglial activity in the cortex in a second subject, and wherein the reference microglial activity in the cortex is measured when said second subject does not receive the treatment using said method, and wherein said second subject is the same or different from said subject.
17. The method according to any one of paragraphs 1 to 16, wherein said method results in a TPP1 activity in the brain of said subject that is at least 2%, 3%, 5%, 6%, 7%, 8%, 9%, 10%, 12%, 15%, 17%, 20%, 25%, 30%, 35%, 40%, 45%, or 50% higher than a reference TPP1 activity in the brain of a second subject, wherein the reference TPP1 activity in the brain is measured when said second subject does not receive the treatment using said method, and wherein said second subject is the same or different from said subject.
18. The method according to any one of paragraphs 1 to 17, wherein said rAAV is administered in a therapeutically effective amount.
19. The method according to any one of paragraphs 1 to 18, wherein said subject is human.
20. The method according to any one of paragraphs 1 to 19, wherein the coding sequence of (c) is a codon optimized human CLN2, which is at least 70% identical to the native human coding sequence of SEQ ID NO: 2.
21. The method according to any one of paragraphs 1 to 20, wherein the coding sequence of (c) is SEQ ID NO: 3.
22. The method according to any one of paragraphs 1 to 21, wherein the rAAV capsid is an AAV9 or a variant thereof.
23. The method according to any one of paragraphs 1 to 22, wherein the promoter is a chicken beta actin (CBA) promoter.
24. The method according to any one of paragraphs 1 to 23, wherein the promoter is a hybrid promoter comprising a CBA promoter sequence and cytomegalovirus enhancer elements.
25. The method according to any of paragraphs 1 to 24, wherein the AAV 5′ ITR and/or AAV3′ ITR is from AAV2.
26. The method according to any of paragraphs 1 to 25, wherein the vector genome further comprises a polyA.
27. The method according to paragraph 26, wherein the polyA is a synthetic polyA or from bovine growth hormone (bGH), human growth hormone (hGH), SV40, rabbit β-globin (RGB), or modified RGB (mRGB).
28. The method according to any of paragraphs 1 to 27, wherein the vector genome further comprises an intron.
29. The method according to paragraph 28, wherein the intron is from CBA, human beta globin, IVS2, SV40, bGH, alpha-globulin, beta-globulin, collagen, ovalbumin, or p53.
30. The method according to any of paragraphs 1 to 29, wherein the vector genome further comprises an enhancer.
31. The method according to paragraph 30, wherein the enhancer is a CMV enhancer, an RSV enhancer, an APB enhancer, ABPS enhancer, an alpha mic/bik enhancer, TTR enhancer, en34, ApoE.
32. The method according to any of paragraphs 1 to 31, wherein the vector genome is about 3 kilobases to about 5.5 kilobases in size.
33. The method according to any of paragraphs 1 to 32, wherein the vector genome is about 4 kilobases in size.
34. The method according to any of paragraphs 1 to 33, wherein the rAAV is manufactured using a method comprising growing in suspension culture a suspension cell line that is capable of producing the rAAV.
35. The method according to paragraph 34, wherein said suspension cell line is HEK293 suspension cell line.
36. A pharmaceutical composition comprising:
(a) a recombinant adeno-associated virus (rAAV),
(b) sodium chloride,
(c) magnesium chloride,
(d) potassium chloride,
(e) dextrose,
(f) poloxamer 188,
(g) sodium phosphate monobasic, and
(h) sodium phosphate dibasic,
wherein said recombinant adeno-associated virus (rAAV) comprises an AAV capsid and a vector genome packaged therein, and wherein said vector genome comprising: (i) an AAV 5′ inverted terminal repeat (ITR) sequence; (ii) a promoter; (iii) a CLN2 coding sequence encoding a human TPP1; and (iv) an AAV 3′ ITR.
37. The pharmaceutical composition according to paragraph 36 further comprising calcium chloride.
38. The pharmaceutical composition according to paragraph 37, wherein said sodium chloride, said magnesium chloride, said potassium chloride, said dextorse, said poloxamer 188, said sodium phosphate monobasic, said sodium phosphate dibasic, and said calcium chloride are each in anhydrous, monohydrate, dihydrate, 3-hydrate, 4-hydrate, 5-hydrate, 6-hydrate, 7-hydrate, 8-hydrate, 9-hydrate, or 10-hydrate form.
39. The pharmaceutical composition according to any one of paragraphs 36 to 38 comprising:
(a) said rAAV,
(b) sodium chloride at a concentration of about 8.77 g/L,
(c) magnesium chloride 6-hydrate, at a concentration of about 0.244 g/L,
(d) potassium chloride at a concentration of about 0.224 g/L,
(e) calcium chloride dihydrate at a concentration of about 0.206 g/L,
(f) dextorse anhydrous at a concentration of about 0.793 g/L,
(g) poloxamer 188 at a concentration of about 0.001% (volume/volume),
(h) sodium phosphate monobasic monohydrate at a concentration of about 0.0278 g/L, and
(i) sodium phosphate dibasic anhydrous at a concentration of about 0.114 g/L.
40. The pharmaceutical composition according to any one of paragraphs 36 to 39, wherein the vector genome concentration (VGC) of the pharmaceutical composition is about 1×10¹¹ GC/mL, about 3×10¹¹ GC/mL, about 6×10¹¹ GC/mL, about 1×10¹² GC/mL, about 3×10¹² GC/mL, about 6×10¹² GC/mL, about 1×10¹³ GC/mL, about 2×10¹³ GC/mL, about 3×10¹³ GC/mL, about 4×10¹³ GC/mL, about 5×10¹³ GC/mL, about 6×10¹³ GC/mL, about 7×10¹³ GC/mL, about 8×10¹³ GC/mL, about 9×10¹³ GC/mL, or about 1×10¹⁴ GC/mL, about 3×10¹⁴ GC/mL, about 6×10¹⁴ GC/mL, or about 1×10¹⁵ GC/mL.
41. The pharmaceutical composition according to any one of paragraphs 36 to 40, wherein the pH of the pharmaceutical composition is in a range from about 6.0 to about 9.0.
42. The pharmaceutical composition according to paragraph 41, wherein the pH of the pharmaceutical composition is about 7.4.
43. The pharmaceutical composition according to any one of paragraphs 36 to 42, wherein said rAAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant rAAV in a reference pharmaceutical composition.
44. The pharmaceutical composition according to paragraph 43, wherein the stability of said rAAV is determined by

(a) the infectivity of rAAV,

(b) the levels of aggregation of rAAV, or

(c) the levels of free DNA released by the rAAV.

45. The pharmaceutical composition according to any one of paragraphs 36 to 44, wherein the pharmaceutical composition is a liquid composition.
46. The pharmaceutical composition according to any one of paragraphs 36 to 44, wherein the pharmaceutical composition is a frozen composition.
47. The pharmaceutical composition according to any one of paragraphs 36 to 44, wherein the pharmaceutical composition is a lyophilized composition or a reconstituted lyophilized composition.
48. The pharmaceutical composition according to any one of paragraphs 36 to 47, wherein the pharmaceutical composition has a property that is suitable for intracerebroventricular (ICV), intracisternal (IC), intrathecal-lumbar, intracranial, intravenous, intravascular, intraarterial, intramuscular, intraocular, intramuscular, subcutaneous, or intradermal administration.
49. The pharmaceutical composition according to any one of paragraphs 36 to 48, wherein the coding sequence of (iii) is a codon optimized human CLN2, which is at least 70% identical to the native human coding sequence of SEQ ID NO: 2.
50. The pharmaceutical composition according to any one of paragraphs 36 to 49, wherein the coding sequence of (iii) is SEQ ID NO: 3.
51. The pharmaceutical composition according to any one of paragraphs 36 to 50, wherein the rAAV capsid is an AAV9 or a variant thereof.
52. The pharmaceutical composition according to any one of paragraphs 36 to 51, wherein the promoter is a chicken beta actin (CBA) promoter.
53. The pharmaceutical composition according to any one of paragraphs 36 to 52, wherein the promoter is a hybrid promoter comprising a CBA promoter sequence and cytomegalovirus enhancer elements.
54. The pharmaceutical composition according to any one of paragraphs 36 to 53, wherein the AAV 5′ ITR and/or AAV3′ ITR is from AAV2.
55. The pharmaceutical composition according to any one of paragraphs 36 to 54, wherein the vector genome further comprises a polyA.
56. The pharmaceutical composition according to any one of paragraphs 36 to 55, wherein the polyA is a synthetic polyA or from bovine growth hormone (bGH), human growth hormone (hGH), SV40, rabbit β-globin (RGB), or modified RGB (mRGB).
57. The pharmaceutical composition according to any one of paragraphs 36 to 56, wherein the vector genome further comprises an intron.
58. The pharmaceutical composition according to any one of paragraphs 36 to 57, wherein the intron is from CBA, human beta globin, IVS2, SV40, bGH, alpha-globulin, beta-globulin, collagen, ovalbumin, or p53.
59. The pharmaceutical composition according to any one of paragraphs 36 to 58, wherein the vector genome further comprises an enhancer.
60. The pharmaceutical composition according to any one of paragraphs 36 to 59, wherein the enhancer is a CMV enhancer, an RSV enhancer, an APB enhancer, ABPS enhancer, an alpha mic/bik enhancer, TTR enhancer, en34, ApoE.
61. The pharmaceutical composition according to any one of paragraphs 36 to 60, wherein the vector genome is about 3 kilobases to about 5.5 kilobases in size.
62. The pharmaceutical composition according to any one of paragraphs 36 to 61, wherein the vector genome is about 4 kilobases in size.
63. The pharmaceutical composition according to any one of paragraphs 36 to 62, wherein the rAAV is manufactured using a method comprising growing in suspension culture a suspension cell line that is capable of producing the rAAV.
64. A method of treating CLN2 Batten disease in a subject comprising administering to said subject the pharmaceutical composition according to any one of paragraphs 36 to 63.
65. The method according to paragraph 64, wherein said pharmaceutical composition is administered in a therapeutically effective amount.
66. The method according to any one of paragraphs 64 to 65, wherein said subject is human.
67. A kit comprising one or more containers and instructions for use, wherein the one or more containers comprise the pharmaceutical composition according to any one of paragraphs 36 to 63.

### BRIEF DESCRIPTION OF THE DRAWINGS

FIG. 1A is a schematic representation of the AAV.CB7.CI.hTPP1co.RBG vector genome. ITR represents an AAV2 inverted terminal repeat. CB7 represents a chicken beta actin promoter with cytomegalovirus enhancer. RBG PolyA represents a rabbit beta globin polyadenylation signal.

FIG. 1B provides a map of the production plasmid of the AAV.hTPP1co vector.

FIG. 1C provides a map of the AAV cis plasmid construct. ITR: inverted terminal repeat; CMV IE promoter: cytomegalovirus immediate-early promoter; CB promoter: chicken β-actin promoter Chicken β-actin intron; hCLN2: Human CLN2 cDNA; Rabbit globin poly A: Rabbit beta-globin polyadenylation signal; Kan-r: kanamycin resistance gene.

FIG. 1D provides a map of the AAV trans packaging plasmid construct.

FIG. 1E provides a map of the adenovirus helper plasmid.

FIG. 2 demonstrates that survival increased in AAV9.CB7.hCLN2-treated TPP1^{mlJ} KO mice.

FIG. 3 demonstrates that TPP1 activity increased in the brain of AAV9.CB7.hCLN2-treated TPP1^{mlJ} KO mice. *p<0.05; **p<0.01 compared to untreated TPP1^{mlJ} KO mice using the Wilcoxon test. Individual values presented alongside mean and SEM.

FIG. 4 demonstrates that TPP1 activity increased in the spinal cord of AAV9.CB7.hCLN2-treated TPP1^{mlJ} KO mice. **p<0.01 compared to untreated TPP1^{mlJ} KO mice using the Wilcoxon test. Individual values presented alongside mean and SEM.

FIG. 5 demonstrates that astrocytosis decreased in AAV9.CB7.hCLN2-treated TPP1^{mlJ} KO mice. *p<0.05; **p<0.01 compared to untreated TPP1^{mlJ} KO mice using a one-way ANOVA. Individual values presented alongside mean and SEM.

FIG. 6 demonstrates that microglian activation decreased in AAV9.CB7.hCLN2-treated TPP1^{mlJ} KO mice. *p<0.05; **p<0.01 compared to untreated TPP1^{mlJ} KO mice using a one-way ANOVA. Individual values presented alongside mean and SEM.

FIG. 7 demonstrates that hepatic TPP1 activity increased in AAV9.CB7.hCLN2-treated TPP1^{mlJ} KO mice. *p<0.05; **p<0.01 compared to untreated TPP1^{mlJ} KO mice using the Wilcoxon test. Individual values presented alongside mean and SEM.

FIG. 8 demonstrates that serum TPP1 activity increased in AAV9.CB7.hCLN2-treated TPP1^{mlJ} KO mice. *p<0.05; **p<0.01 compared to untreated TPP1^{mlJ} KO mice using the Wilcoxon test. Individual values presented alongside mean and SEM.

FIG. 9 demonstrates that AAV9.CB7.hCLN2 therapy provided gain of survival in KO animals after treatment with HD at 1-month of age. All surviving animals were necropsied 26 weeks post-ICV and no differences were observed between controls and LD treated KO. WT males (black circle), WT females (grey square), and HD KO males (black X) are overlaid in the plot because no mortality has been observed in these 3 groups. WT: Wild-Type; KO: TPP KO; M: Male Mice, F: Female Mice, LD: Low dose (3×10⁹GC/animal); HD: High dose (3×10¹¹ GC/animal).

FIGS. 10A-10C show correction of astrocytosis. A. Brainstem. B. Hippocampus. C. Cortex. Results are an average number of astrocytes per ×20 power field. “KO PBS treated” (no ICV) are 3-month-old animals (pre-disease onset) coming from the natural history study (W2553A). P-value by unpaired Mann Whitney test. WT: Wild-Type; KO: TPP1^{mlJ} KO; and HD: High dose (3×10¹¹ GC/animal).

FIG. 11 shows survival data. All vehicle-treated TPP1^{mlJ} KO mice were found dead or were humanely euthanized before the age of 19 weeks, whereas 67% of AAV9.CB7.hCLN2-treated females (3×10¹¹ GC/animal) and 57% of AAV9.CB7.hCLN2-treated males (3×10¹¹ GC/animal) were alive at the scheduled 23-week endpoint. KO3e11 M: knock out AAV9.CB7.HCLN2-treated males (3×10¹¹); KO3e11 F: knock out AAV9.CB7.hCLN2-treated females (3×10¹¹); KO M: knock out vehicle-treated males; KO F: knock out vehicle-treated females; WT M: Wild-Type males; WT F: Wild-Type females.

FIGS. 12A-12B demonstrate AAV9.CB7.hCLN2 increased survival in TPP1^{mlJ} KO mice. Groups of TPP1^{mlJ} KO mice were administered doses of 0, 1.25×10¹⁰, 5×10¹⁰, 2×10¹¹ or 8.5×10¹¹ GC/animal. An additional group of WT mice were untreated (study ongoing).

FIGS. 13A-13B demonstrate that AAV9.CB7.hCLN2 increased TPP1 activity in the brain of TPP1^{mlJ} KO mice. Groups of TPP1^{mlJ} KO mice were administered doses of 0, 1.25×10¹⁰, 5×10¹⁰, 2×10¹¹ or 8.5×10¹¹ GC/animal. An additional group of WT mice were untreated. At 13 weeks of age (Week 9), animals were euthanized and the right hemisphere of brain analyzed for TPP1 activity for A) males and B) females. *p≤0.05; **p≤0.01. P-values are obtained using the 2-sided exact Wilcoxon rank-sum test, comparing each dosed group against an independent control group, with the null hypothesis of no difference between the two groups.

FIG. 14 demonstrates that AAV9.CB7.hCLN2 increased TPP1 activity in the spinal cord of TPP1^{mlJ} KO mice. Groups of TPP1^{mlJ} KO mice were administered doses of 0, 1.25×10¹⁰, 5×10¹⁰, 2×10¹¹ or 8.5×10¹¹ GC/animal. An additional group of WT mice were untreated. At 13 weeks of age (Week 9), animals were euthanized and the spinal cord (thoracic) analyzed for TPP1 activity, black represents males, grey represents females. Male and female data combined due to the limited number of samples analyzed. *p≤0.05; **p≤0.01. P-values are obtained using the 2-sided exact Wilcoxon rank-sum test, comparing each dosed group against an independent control group, with the null hypothesis of no difference between the two groups.

FIGS. 15A-15B demonstrate that AAV9.CB7.hCLN2 increased TPP1 activity in the liver of TPP1^{mlJ} KO mice. Groups of TPP1^{mlJ} KO mice were administered doses of 0, 1.25×10¹⁰, 5×10¹⁰, 2×10¹¹ or 8.5×10¹¹ GC/animal. An additional group of WT mice were untreated. At 13 weeks of age (Week 9), animals were euthanized and the liver analyzed for TPP1 activity for A) males and B) females. *p≤0.05; **p≤0.01. P-values are obtained using the 2-sided exact Wilcoxon rank-sum test, comparing each dosed group against an independent control group, with the null hypothesis of no difference between the two groups.

FIGS. 16A-16B demonstrate that AAV9.CB7.hCLN2 increased TPP1 activity in the serum of TPP1^{mlJ} KO mice. Groups of TPP1^{mlJ} KO mice were administered doses of 0, 1.25×10¹⁰, 5×10¹⁰, 2×10¹¹ or 8.5×10¹¹ GC/animal. An additional group of WT mice were untreated. At 13 weeks of age (Week 9), animals were euthanized and blood collected for serum TPP1 activity for A) males and B) females. *p≤0.05; **p≤0.01. P-values are obtained using the 2-sided exact Wilcoxon rank-sum test, comparing each dosed group against an independent control group, with the null hypothesis of no difference between the two groups.

FIGS. 17A-17B demonstrate that AAV9.CB7.hCLN2 decreased astrocytosis in TPP1^{mlJ} KO mice. Groups of TPP1^{mlJ} KO mice were administered doses of 0 (Group 2) 1.25×10¹⁰ (Group 3), 5×10¹⁰ (Group 4), 2×10¹¹ (Group 5) or 8.5×10¹¹ (Group 6) GC/animal. An additional group of WT mice were untreated (Group 1). Sections of brain were stained with GFAP to measure the relative level of immunofluorescence of astrocytes in A) the somatosensory barrel cortex (S1BF) and B) thalamus (ventral posterolateral nucleus [VPL] and ventral posteromedial nucleus [VPM]). *p≤0.05, **p≤0.01, ***p≤0.001.

FIGS. 18A-18B demonstrate that AAV9.CB7.hCLN2 decreased microglial activation in TPP1^{mlJ} KO mice. Groups of TPP1^{mlJ} KO mice were administered doses of 0 (Group 2) 1.25×10¹⁰ (Group 3), 5×10¹⁰ (Group 4), 2×10¹¹ (Group 5) or 8.5×10¹¹ (Group 6) GC/animal. An additional group of WT mice were untreated (Group 1). Sections of brain were stained with CD68 to measure the relative level of immunofluorescence of microglia in A) the somatosensory barrel cortex (S1BF) and B) thalamus (ventral posterolateral nucleus [VPL] and ventral posteromedial nucleus [VPM]).

FIGS. 19A-19B show the TPP1 activity in the serum of C57Bl/6 mice. Groups of C57Bl/6 mice were administered doses of 0, 1.25×10¹⁰, 5×10¹⁰, 2×10¹¹ or 8.5×10¹¹ GC/animal. 4 or 13 weeks after dosing, animals were euthanized and blood collected for serum TPP1 activity for A) males and B) females. *p≤0.05; **p≤0.01 Vs time-matched control group. P-values are obtained using the 2-sided exact Wilcoxon rank-sum test, comparing each dosed group against an independent control group, with the null hypothesis of no difference between the two groups.

FIGS. 20A-20B show the TPP1 activity in the brain of C57Bl/6 mice. Groups of C57Bl/6 mice were administered doses of 0, 1.25×10¹⁰, 5×10¹⁰, 2×10¹¹ or 8.5×10¹¹ GC/animal. 4 or 13 weeks after dosing, animals were euthanized and brain collected for TPP1 activity for A) males and B) females. *p≤0.05; **p≤0.01 Vs time-matched control group. P-values are obtained using the 2-sided exact Wilcoxon rank-sum test, comparing each dosed group against an independent control group, with the null hypothesis of no difference between the two groups.

FIGS. 21A-21B show TPP1 activity in the liver of C57Bl/6 mice. Groups of, C57Bl/6 mice were administered doses of 0, 1.25×10¹⁰, 5×10¹⁰, 2×10¹¹ or 8.5×10¹¹ GC/animal. 4 or 13 weeks after dosing, animals were euthanized and liver collected for TPP1 activity for A) males and B) females. *p≤0.05; **p≤0.01 Vs time-matched control group. P-values are obtained using the 2-sided exact Wilcoxon rank-sum test, comparing each dosed group against an independent control group, with the null hypothesis of no difference between the two groups.

FIGS. 22A-22B show the differences in TPP1 activity and concentration from baseline in serum. Groups of cynomolgus monkeys (n=3/dose) were administered AAV9.CB7.hCLN2 via injection into the cisterna magna (CM) at doses of 0, 3.4×10¹¹, 3.2×10¹² or 2.9×10¹³ genome copies (GC)/animal (dose volume of 1 mL). Blood samples were collected pre-dose (Day −1 or 1) and on Days 4, 8, 11, 15, 18, 22, 25 and 29 for analysis of (A) TPP1 activity and (B) TPP1 concentration. Difference from baseline mean values are presented with standard error of the mean.

FIGS. 23A-23B show the differences in TPP1 activity and concentration from baseline in CSF. Groups of cynomolgus monkeys (n=3/dose) were administered AAV9.CB7.HCLN2 via injection into the cisterna magna (CM) at doses of 0, 3.4×10¹¹, 3.2×10¹² or 2.9×10¹³ genome copies (GC)/animal (dose volume of 1 mL). CSF samples were collected pre-dose (Day −1 or 1) and on Days 4, 8, 11, 15, 18, 22, 25 and 29 for analysis of (A) TPP1 activity and (B) TPP1 concentration. Difference from baseline mean values are presented with standard error of the mean.

FIGS. 24A-24D show TPP1 concentration in the brain areas of (A) frontal cortex, (B) striatum, (C) thalamus and (D) midbrain. Groups of cynomolgus monkeys (n=3/dose) were administered AAV9.CB7.HCLN2 via injection into the cisterna magna (CM) at doses of 0, 3.4×10¹¹, 3.2×10¹² or 2.9×10¹³ genome copies (GC)/animal (dose volume of 1 mL). At necropsy on Day 29, two tissue punches were collected from either the deep (>3 mm; D) or superficial (<3 mm deep; S) areas of (A) frontal cortex, (B) striatum, (C) thalamus and (D) midbrain. Individual values presented alongside mean and standard deviation.

FIGS. 25A-25C show TPP1 concentration in the brain areas of (A) occipital cortex, (B) medulla oblongata and (C) cerebellum. Groups of cynomolgus monkeys (n=3/dose) were administered AAV9.CB7.HCLN2 via injection into the cisterna magna (CM) at doses of 0, 3.4×10¹¹, 3.2×10¹² or 2.9×10¹³ genome copies (GC)/animal (dose volume of 1 mL). At necropsy on Day 29, two tissue punches were collected from either the deep (>3 mm; D) or superficial (<3 mm deep; S) areas of (A) occipital cortex, (B) medulla oblongata and (C) cerebellum. Individual values presented alongside mean and standard deviation.

FIGS. 26A-26B show TPP1 concentration in the spinal cord. Groups of cynomolgus monkeys (n=3/dose) were administered AAV9.CB7.HCLN2 via injection into the cisterna magna (CM) at doses of 0, 3.4×10¹¹, 3.2×10¹² or 2.9×10¹³ genome copies (GC)/animal (dose volume of 1 mL). At necropsy on Day 29, two tissue punches were collected from either the cervical, thoracic or lumbar regions of the spinal cord of (A) TPP1 activity and (B) TPP1 concentration. Individual values presented alongside mean and standard deviation.

FIG. 27 shows temperature profile measured for 2 different fill volumes in Nalgene HDPE BD S bottles.

FIG. 28 shows temperature profiles recorded for 0.6 mL fills in 2 mL cryovials cycled between −80° C. and room temperature or −20° C.

FIG. 29 shows Fast Freeze/Fast Thaw (FF/FT) temperature profile.

FIG. 30 shows Fast Freeze/Fast Thaw (FF/FT) temperature profile (left axis) and Rates for the Shelf and Probes (right axis).

FIG. 31 shows Fast Freeze/Slow Thaw (FF/ST) temperature profile.

FIG. 32 shows Slow Freeze/Fast Thaw (SF/FT) temperature profile.

FIG. 33 shows Slow Freeze/Slow Thaw (SF/ST) temperature profile.

FIG. 34 shows Slow Freeze/Slow Thaw (SF/ST) temperature profile (left axis) and rates for the shelf and probes (right axis).

FIG. 35 shows zoomed-in view of SEC result profiles for AAV9.CB7.HCLN2 in intrathecal buffer.

FIG. 36 shows DLS diameter results for AAV9.CB7.HCLN2 freeze-thaw samples.

FIG. 37 shows Low temperature DSC thermogram for AAV9.CB7.HCLN2 modified Elliott's B formulation buffer.

FIG. 38 is a flow diagram for manufacture of bulk drug substance (upstream).

FIG. 39 is a flow diagram for manufacture of bulk drug substance (downstream).

### DETAILED DESCRIPTION OF THE INVENTION

Provided herein are methods and compositions for treatment of Batten disease. Such compositions include a recombinant adeno-associated virus (rAAV), said rAAV comprising an AAV capsid, and a vector genome packaged therein, said vector genome comprising (a) an AAV 5′ inverted terminal repeat (ITR) sequence; (b) a promoter; (c) a CLN2 coding sequence encoding a human TPP1; (d) an AAV 3′ ITR (See Section I). Also provided herein are methods of treating Batten disease using the rAAV provided herein and related pharmaceutical compositions (See Section II). More specifically, provided herein are methods of treating Batten disease comprising administering to a subject in need thereof the rAAV described herein via more than one route (See Section II). Also provided herein are methods of manufacturing the rAAV described herein using suspension cell culture (See Section III).

I. Recombinant Adeno-associated Virus (rAAV)

In certain embodiments, the AAV9.CB7.hCLN2 provided herein is described in the following embodiments. The methods and compositions described herein involve compositions and methods for delivering a CLN2 nucleic acid sequence encoding human tripeptidyl peptidase1 (TPP1) protein to subjects in need thereof for the treatment of NCL. In one embodiment, such compositions involve codon optimization of the CLN2 coding sequence, such as that shown in SEQ ID NO: 3. It is desirable to increase the efficacy of the product, and thus, increase safety, since a lower dose of reagent may be used. Also encompassed herein are compositions which include the native CLN2 coding sequences, as shown in SEQ ID NO: 2.

The TPP1 gene, also known as CLN2, encodes Tripeptidyl-peptidase 1, a lysosomal serine protease with tripeptidyl-peptidase I activity. It is also thought to act as a non-specific lysosomal peptidase which generates tripeptides from the breakdown products produced by lysosomal proteinases and requires substrates with an unsubstituted N-terminus. As used herein, the terms “TPP1”, “CLN2”, and “Tripeptidyl-peptidase 1” are used interchangeably when referring to the coding sequence. The native nucleic acid sequence encoding human Tripeptidyl-peptidase 1 is reported at NCBI Reference Sequence NM_000391.3 and reproduced here in SEQ ID NO: 2. Two isoforms of human Tripeptidyl-peptidase 1 has been reported as UniProtKB/Swiss-Prot Accessions O14773-1 and O14773-2 (reproduced here as SEQ ID NOs: 1 and 4). Mutations in the CLN2 gene are associated with late-infantile NCL (LINCL) disease.

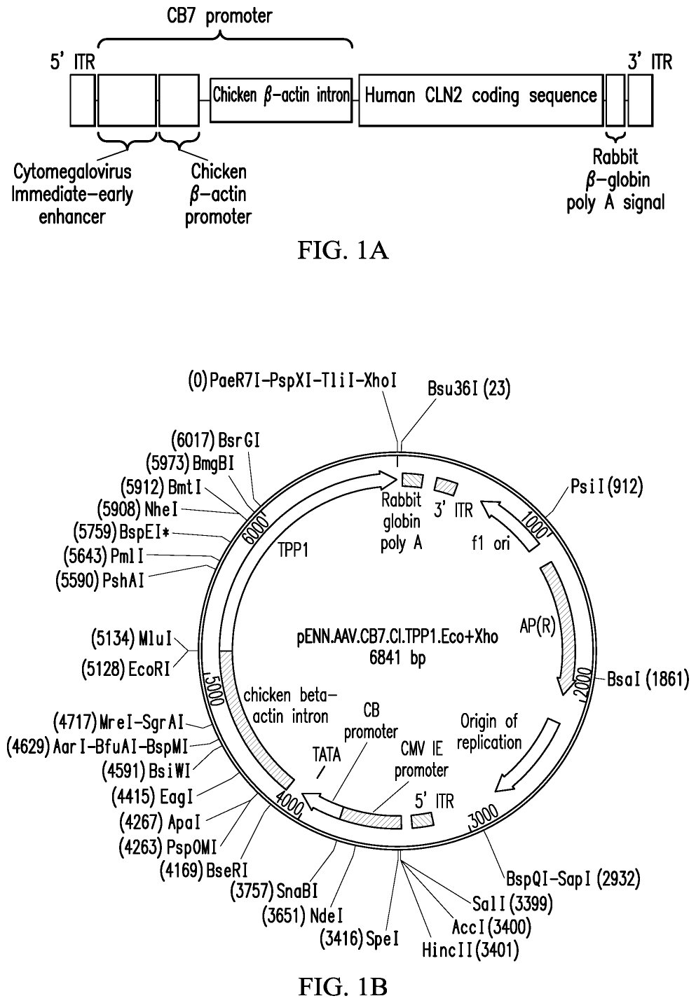

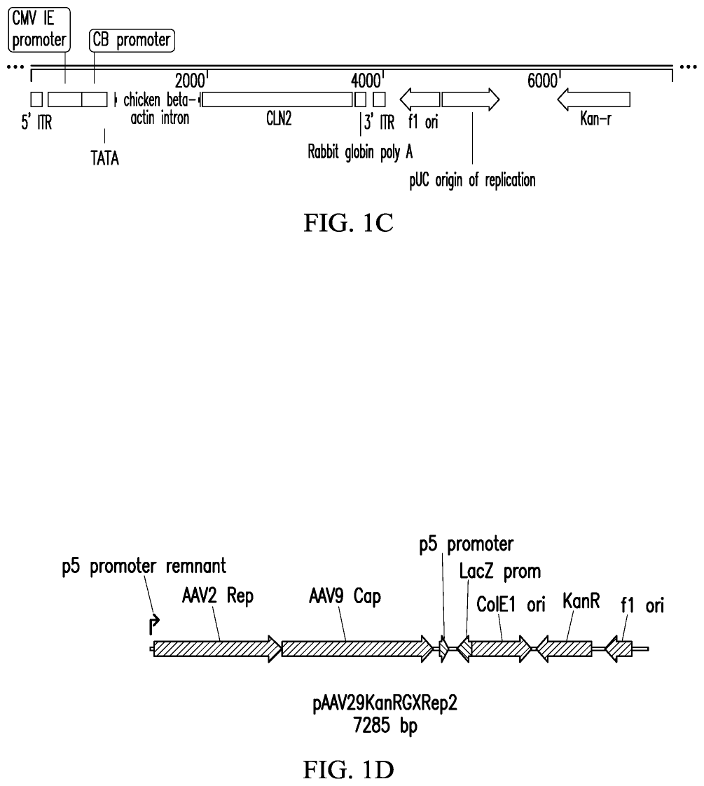

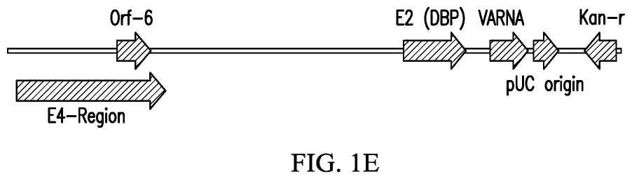

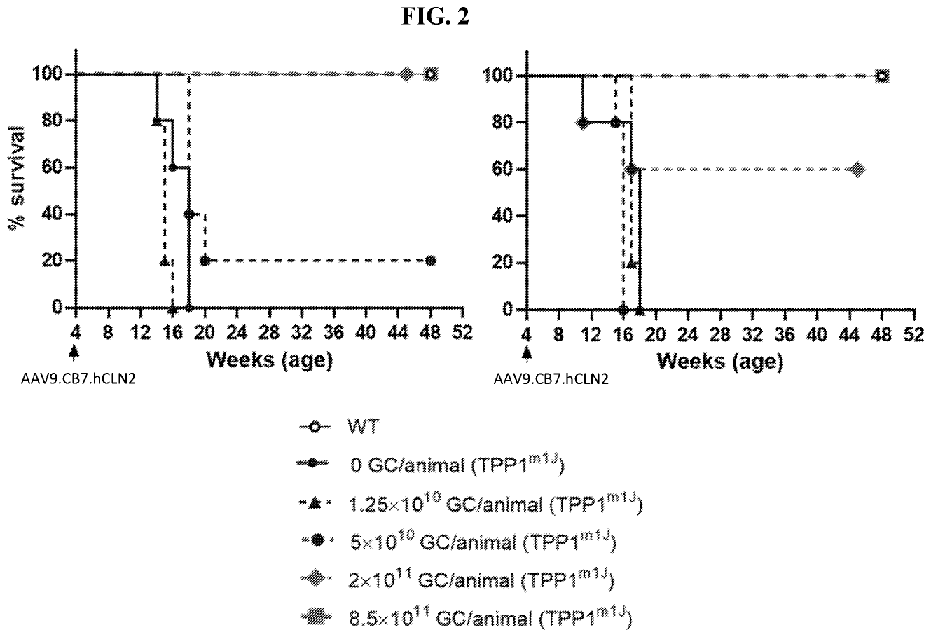

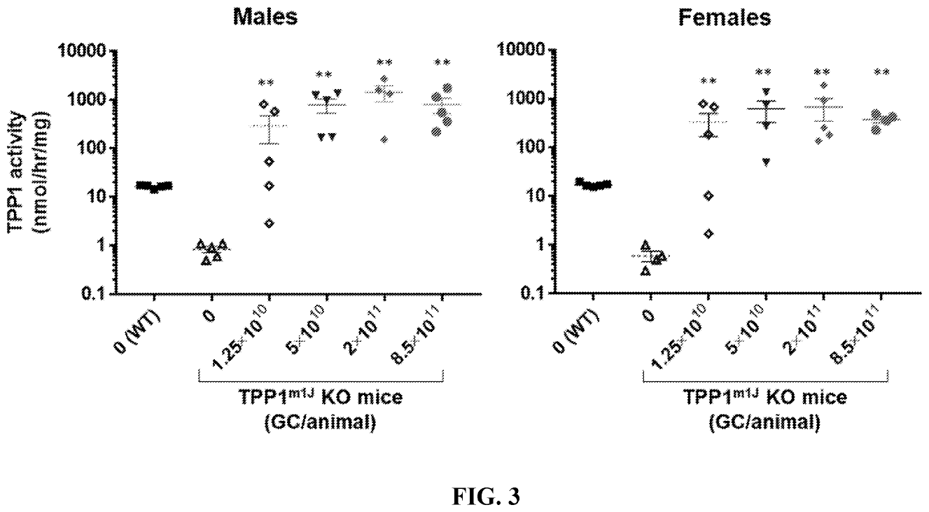

In certain embodiments, AAV.hTPP1co vectors may be designed as described in WO 2018209205A1. In certain embodiments, the human (h) TPP1-encoding optimized cDNA may be custom-designed for optimal codon usage and synthesized. In certain embodiments, the hTPP1co cDNA as reproduced as SEQ ID NO: 3 may be then placed in a transgene expression cassette which was driven by a CB7 promoter, a hybrid between a cytomegalovirus (CMV) immediate early enhancer (C4) and the chicken beta actin promoter, while transcription from this promoter is enhanced by the presence of the chicken beta actin intron (CI) (FIGS. 1A and 1B). In certain embodiments, the polyA signal for the expression cassette is the rabbit beta-globin (RBG) polyA.

In certain embodiments, a 6841 bp production plasmid of AAV.hTPP1co vector (AAV.CB7.CI.hTPP1co.RBG) may be constructed with the hTPP1co expression cassette described herein flanked by AAV2 derived ITRs as well as resistance to Ampicillin as a selective marker (FIG. 1B). In certain embodiments, a similar AAV.hTPP1co production plasmid with resistance to Kanamycin may also be constructed. In certain embodiments, the vectors derived from both plasmids may be single-stranded DNA genome with AAV2 derived ITRs flanking the hTPP1co expression cassette described herein.

In certain embodiments, the AAV.hTPP1co vectors may be made by triple transfection and formulated in excipient consisting of phosphate-buffered saline (PBS) containing and 0.001% Pluronic F68 (PF68). See, e.g. Mizukami, Hiroaki, et al., A Protocol for AAV vector production and purification, Diss. Di-vision of Genetic Therapeutics, Center for Molecular Medicine, 1998. In certain embodiments, the genome titers of the vector produced may be determined via droplet digital PCR (ddPCR). See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Described herein is an exemplary AAV.hTPP1co vector, which is sometimes referred to herein as AAV9.CB7.hCLN2. The use of these terms is interchangeable. In addition, where, in one embodiment, the AAV9.CB7.hCLN2 vector is referred to, alternate embodiments are contemplated utilizing the components as described herein.

In certain embodiments of this invention, a subject has neuronal ceroid lipofuscinosis (NCL), for which the components, compositions and methods of this invention are designed to treat. As used herein, the term “subject” as used herein means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

The neuronal ceroid-lipofuscinoses (NCLs) are a group of inherited, neurodegenerative, lysosomal storage disorders characterized by progressive intellectual and motor deterioration, seizures, and early death. Visual loss is a feature of most forms. Clinical phenotypes have been characterized traditionally according to the age of onset and order of appearance of clinical features into infantile, late-infantile, juvenile, adult, and Northern epilepsy (also known as progressive epilepsy with mental retardation [EPMR]). There is however genetic and allelic heterogeneity; a proposed new nomenclature and classification system has been developed to take into account both the responsible gene and the age at disease onset; for example, CLN2 disease, classic late infantile. The first symptoms typically appear between age two and four years, usually starting with epilepsy, followed by regression of developmental milestones, myoclonic ataxia, and pyramidal signs. Visual impairment typically appears at age four to six years and rapidly progresses to light/dark awareness only. Life expectancy ranges from age six years to early teenage. As used herein, the term “Batten disease” is used to refer to a CLN2 disease, which is used interchangeably with “NCL”.

In one aspect, a codon optimized, engineered nucleic acid sequence encoding human (h) TPP1 is provided. In certain embodiments, an engineered human (h) TPP1 cDNA is provided herein (as SEQ ID NO: 3), which was designed to maximize translation as compared to the native TPP1 sequence (SEQ ID NO: 2). Preferably, the codon optimized TPP1 coding sequence has less than about 80% identity, preferably about 75% identity or less to the full-length native TPP1 coding sequence (SEQ ID NO: 2). In one embodiment, the codon optimized TPP1 coding sequence has about 74% identity with the native TPP1 coding sequence of SEQ ID NO: 2. In one embodiment, the codon optimized TPP1 coding sequence is characterized by improved translation rate as compared to native TPP1 following AAV-mediated delivery (e.g., rAAV). In one embodiment, the codon optimized TPP1 coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the full length native TPP1 coding sequence of SEQ ID NO: 2. In one embodiment, the codon optimized nucleic acid sequence is a variant of SEQ ID NO: 3. In another embodiment, the codon optimized nucleic acid sequence a sequence sharing about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or greater identity with SEQ ID NO: 3. In one embodiment, the codon optimized nucleic acid sequence is SEQ ID NO: 3. In another embodiment, the nucleic acid sequence is codon optimized for expression in humans. In other embodiments, a different TPP1 coding sequence is selected.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available [e.g., BLAST, ExPASy; ClustalO; FASTA; using, e.g., Needleman-Wunsch algorithm, Smith-Waterman algorithm]. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

Codon-optimized coding regions can be designed by various different methods. This optimization may be performed using methods which are available on-line (e.g., GeneArt), published methods, or a company which provides codon optimizing services, e.g., DNA2.0 (Menlo Park, Calif.). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

By “engineered” is meant that the nucleic acid sequences encoding the TPP1 protein described herein are assembled and placed into any suitable genetic element, e.g., naked DNA, phage, transposon, cosmid, episome, etc., which transfers the TPP1 sequences carried thereon to a host cell, e.g., for generating non-viral delivery systems (e.g., RNA-based systems, naked DNA, or the like) or for generating viral vectors in a packaging host cell and/or for delivery to a host cells in a subject. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

As used herein, the term “host cell” may refer to the packaging cell line in which a recombinant AAV is produced from a production plasmid. In the alternative, the term “host cell” may refer to any target cell in which expression of the coding sequence is desired. Thus, a “host cell,” refers to a prokaryotic or eukaryotic cell that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to the cells employed to generate and package the viral vector or recombinant virus. In other embodiments herein, the term “host cell” refers to cultures of CNS cells of various mammalian species for in vitro assessment of the compositions described herein. Still in other embodiments, the term “host cell” is intended to reference the brain cells of the subject being treated in vivo for Batten disease. Such host cells include epithelial cells of the CNS including ependyma, the epithelial lining of the brain ventricular system. Other host cells include neurons, astrocytes, oligoedendrocytes, and microglia.

As used herein, the term “treatment” or “treating” is defined encompassing administering to a subject one or more compounds or compositions described herein for the purposes of amelioration of one or more symptoms of Batten disease. “Treatment” can thus include one or more of reducing onset or progression of neuronal ceroid lipofuscinosis (NCL), preventing disease, reducing the severity of the disease symptoms, or retarding their progression, including the progression of blindness, removing the disease symptoms, delaying onset of disease or monitoring progression of disease or efficacy of therapy in a given subject.

In one embodiment, the nucleic acid sequence encoding TPP1 further comprises a nucleic acid encoding a tag polypeptide covalently linked thereto. The tag polypeptide may be selected from known “epitope tags” including, without limitation, a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, a flag tag polypeptide, and a maltose binding protein tag polypeptide.

In another aspect, an expression cassette comprising a nucleic acid sequence that encodes TPP1 is provided. In one embodiment, the sequence is a codon optimized sequence. In another embodiment, the codon optimized nucleic acid sequence is SEQ ID NO: 3 encoding human TPP1.

As used herein, an “expression cassette” refers to a nucleic acid molecule which comprises the coding sequences for TPP1 protein, promoter, and may include other regulatory sequences therefor, which cassette may be packaged into the capsid of a viral vector (e.g., a viral particle). Typically, such an expression cassette for generating a viral vector contains the CLN2 sequences described herein flanked by packaging signals of the viral genome and other expression control sequences such as those described herein. For example, for an AAV viral vector, the packaging signals are the 5′ inverted terminal repeat (ITR) and the 3′ ITR. When packaged into the AAV capsid, the ITRs in conjunction with the expression cassette may be referred to herein as the “recombinant AAV (rAAV) genome” or “vector genome”. In one embodiment, an expression cassette comprises a codon optimized nucleic acid sequence that encodes TPP1 protein. In one embodiment, the cassette provides the codon optimized CLN2 operatively associated with expression control sequences that direct expression of the codon optimized nucleic acid sequence that encodes TPP1 in a host cell.

In another embodiment, an expression cassette for use in an AAV vector is provided. In that embodiment, the AAV expression cassette includes at least one AAV inverted terminal repeat (ITR) sequence. In another embodiment, the expression cassette comprises 5′ ITR sequences and 3′ ITR sequences. In one embodiment, the 5′ and 3′ ITRs flank the codon optimized nucleic acid sequence that encodes TPP1, optionally with additional sequences which direct expression of the codon optimized nucleic acid sequence that encodes TPP1 in a host cell. Thus, as described herein, a AAV expression cassette is meant to describe an expression cassette as described above flanked on its 5′ end by a 5′AAV inverted terminal repeat sequence (ITR) and on its 3′ end by a 3′ AAV ITR. Thus, this rAAV genome contains the minimal sequences required to package the expression cassette into an AAV viral particle, i.e., the AAV 5′ and 3′ ITRs. The AAV ITRs may be obtained from the ITR sequences of any AAV, such as described herein. These ITRs may be of the same AAV origin as the capsid employed in the resulting recombinant AAV, or of a different AAV origin (to produce an AAV pseudotype). In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, the AAV vector genome comprises an AAV 5′ ITR, the TPP1 coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used. Each rAAV genome can be then introduced into a production plasmid.

As used herein, the term “regulatory sequences”, “transcriptional control sequence” or “expression control sequence” refers to DNA sequences, such as initiator sequences, enhancer sequences, and promoter sequences, which induce, repress, or otherwise control the transcription of protein encoding nucleic acid sequences to which they are operably linked.

As used herein, the term “operably linked” or “operatively associated” refers to both expression control sequences that are contiguous with the nucleic acid sequence encoding the TPP1 and/or expression control sequences that act in trans or at a distance to control the transcription and expression thereof.

In one aspect, a vector comprising any of the expression cassettes described herein is provided. As described herein, such vectors can be plasmids of variety of origins and are useful in certain embodiments for the generation of recombinant replication defective viruses as described further herein.

A “vector” as used herein is a nucleic acid molecule into which an exogenous or heterologous or engineered nucleic acid transgene may be inserted which can then be introduced into an appropriate host cell. Vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and (primarily in yeast and bacteria) “artificial chromosomes.” Certain plasmids are described herein.

In one embodiment, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference. Such non-viral TPP1 vector may be administered by the routes described herein. The viral vectors, or non-viral vectors, can be formulated with a physiologically acceptable carrier for use in gene transfer and gene therapy applications.

In another embodiment, the vector is a viral vector that comprises an expression cassette described therein. “Virus vectors” are defined as replication defective viruses containing the exogenous or heterologous CLN2 nucleic acid transgene. In one embodiment, an expression cassette as described herein may be engineered onto a plasmid which is used for drug delivery or for production of a viral vector. Suitable viral vectors are preferably replication defective and selected from amongst those which target brain cells. Viral vectors may include any virus suitable for gene therapy, including but not limited to adenovirus; herpes virus; lentivirus; retrovirus; parvovirus, etc. However, for ease of understanding, the adeno-associated virus is referenced herein as an exemplary virus vector.

A “replication-defective virus” or “viral vector” refers to a synthetic or recombinant viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In another embodiment, a recombinant adeno-associated virus (rAAV) vector is provided. The rAAV compromises an AAV capsid, and a vector genome packaged therein.

The vector genome comprises, in one embodiment: (a) an AAV 5′ inverted terminal repeat (ITR) sequence; (b) a promoter; (c) a coding sequence encoding a human TPP1; and (d) an AAV 3′ ITR. In another embodiment, the vector genome is the expression cassette described herein. In one embodiment, the CLN2 sequence encodes a full length TPP1 protein. In one embodiment, the TPP1 sequence is the protein sequence of SEQ ID NO: 1. In another embodiment, the coding sequence is SEQ ID NO: 3 or a variant thereof.

Adeno-associated virus (AAV), a member of the Parvovirus family, is a small nonenveloped, icosahedral virus with single-stranded linear DNA genomes of 4.7 kilobases (kb) to 6 kb. Among known AAV serotypes are AAV1, AAV2, AAV3, AAV4, AAV5, AAV6, AAV7, AAV8, AAV9 and others. The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

Fragments of AAV may be readily utilized in a variety of vector systems and host cells. Among desirable AAV fragments are the cap proteins, including the vp1, vp2, vp3 and hypervariable regions, the rep proteins, including rep 78, rep 68, rep 52, and rep 40, and the sequences encoding these proteins. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a novel AAV sequence of the invention (e.g., a fragment of a vp1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. In one embodiment, a vector contains AAV9 cap and/or rep sequences. See, U.S. Pat. No. 7,906,111, which is incorporated by reference herein.

In one embodiment, an AAV vector having AAV9 capsid characterized by the amino acid sequence of SEQ ID NO: 6, is provided herein, in which a nucleic acid encoding a classic late infantile neuronal ceroid lipofuscinosis 2 (CLN2) gene under control of regulatory sequences directing expression thereof in patients in need thereof.

As used herein, an “AAV9 capsid” is characterized by DNAse-resistant particle which is an assembly of about 60 variable proteins (vp) which are typically expressed as alternative splice variants resulting in proteins of different length of SEQ ID NO: 6. See also Genbank Accession No. AAS99264.1, which is incorporated herein by reference. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809. The amino acid sequence is reproduced in SEQ ID NO: 6 and the coding sequence is reproduced in SEQ ID NO: 7. In one embodiment, the AAV9 capsid includes a capsid encoded by SEQ ID NO: 7, or a sequence sharing at least about 90%, 95%, 95%, 98% or 99% identity therewith.

The largest protein, vp1, is generally the full-length of the amino acid sequence of SEQ ID NO: 6 (aa 1-736 of SEQ ID NO: 6). In certain embodiments, the AAV9 vp2 protein has the amino acid sequence of 138 to 736 of SEQ ID NO: 6. In certain embodiments, the AAV9 vp3 has the amino acid sequence of 203 to 736 of SEQ ID NO: 6. In certain embodiments, the vp 1, 2 or 3 proteins may be have truncations (e.g., 1 or more amino acids at the N-terminus or C-terminus). An AAV9 capsid is composed of about 60 vp proteins, in which vp1, vp2 and vp3 are present in a ratio of about 1 vp, to about 1 vp2, to about 10 to 20 vp3 proteins within the assembled capsid. This ratio may vary depending upon the production system used. In certain embodiments, an engineered AAV9 capsid may be generated in which vp2 is absent.

It is within the skill in the art to design nucleic acid sequences encoding this AAV9 capsid, including DNA (genomic or cDNA), or RNA (e.g., mRNA). In certain embodiments, the nucleic acid sequence encoding the AAV9 vp1 capsid protein is provided in SEQ ID NO: 7. In other embodiments, a nucleic acid sequence of 70% to 99.9% identity to SEQ ID NO: 7 may be selected to express the AAV9 capsid. In certain other embodiments, the nucleic acid sequence is at least about 75% identical, at least 80% identical, at least 85%, at least 90%, at least 95%, at least 97% identical, or at least 99% to 99.9% identical to SEQ ID NO: 7.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics, Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another Glade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321. AAV9 is further characterized by being within Clade F. Other Clade F AAV include AAVhu31 and AAVhu32.

As used herein, relating to AAV, the term variant means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV9 capsid. When determining the percent identity of an AAV capsid, the comparison may be made over any of the variable proteins (e.g., vp1, vp2, or vp3). In one embodiment, the AAV capsid shares at least 95% identity with the AAV9 over the vp1, vp2 or vp3.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/9 and AAV2/rh.10 are exemplary pseudotyped vectors.

In another embodiment, a self-complementary AAV is used. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g. a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence with which the protein or nucleic acid in question is not found in the same relationship to each other in nature.

In still another embodiment, the expression cassette, including any of those described herein is employed to generate a recombinant AAV genome.

In one embodiment, the expression cassette described herein is engineered into a suitable genetic element (vector) useful for generating viral vectors and/or for delivery to a host cell, e.g., naked DNA, phage, transposon, cosmid, episome, etc., which transfers the CLN2 sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

For packaging an expression cassette or rAAV genome or production plasmid into virions, the ITRs are the only AAV components required in cis in the same construct as the expression cassette. In one embodiment, the coding sequences for the replication (rep) and/or capsid (cap) are removed from the AAV genome and supplied in trans or by a packaging cell line in order to generate the AAV vector.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In a specific embodiment, the producer cell line or packaging cell line is a suspension cell line such that the AAV viral vectors described herein can be manufactured by growing the producer cell line or packaging cell line in suspension culture. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

In yet another system, the expression cassette flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety.

In one aspect, provided herein is a method of manufacturing an rAAV described herein, comprising growing in suspension culture a suspension cell line that is capable of producing the rAAV.

In certain embodiments, the suspension cell line is derived from an adherent cell line by adaptation of cells into suspension culture using serum-free and animal component-free culture medium. In a specific embodiment, the suspension cell line is HEK293 suspension cell line.

The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, N.Y. (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

“Plasmids” generally are designated herein by a lower case p preceded and/or followed by capital letters and/or numbers, in accordance with standard naming conventions that are familiar to those of skill in the art. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

In one embodiment, the production plasmid is that described herein, or as described in WO2012/158757, which is incorporated herein by reference. Various plasmids are known in the art for use in producing rAAV vectors, and are useful herein. The production plasmids are cultured in the host cells which express the AAV cap and/or rep proteins. In the host cells, each rAAV genome is rescued and packaged into the capsid protein or envelope protein to form an infectious viral particle.

In one aspect, a production plasmid comprising an expression cassette described above is provided. In one embodiment, the production plasmid is that shown in FIG. 1B. This plasmid is used in the examples for generation of the rAAV-human codon optimized TPP1 vector. Such a plasmid is one that contains a 5′ AAV ITR sequence; a selected promoter; a polyA sequence; and a 3′ ITR; additionally, it also contains an intron sequence, such as the chicken beta-actin intron. An exemplary schematic is shown in FIG. 1A. In a further embodiment, the intron sequence keeps the rAAV vector genome with a size between about 3 kilobases (kb) to about 6 kb, about 4.7 kb to about 6 kb, about 3 kb to about 5.5 kb, or about 4.7 kb to 5.5 kb. An example of a production plasmid which includes the TPP1 encoding sequence can be found in SEQ ID NO: 5. In another embodiment, the production plasmid is modified to optimized vector plasmid production efficiency. Such modifications include addition of other neutral sequences, or inclusion of a lambda stuffer sequence to modulate the level of supercoil of the vector plasmid. Such modifications are contemplated herein. In other embodiments, terminator and other sequences are included in the plasmid.

In certain embodiments, the rAAV expression cassette, the vector (such as rAAV vector), the virus (such as rAAV), and/or the production plasmid comprises AAV inverted terminal repeat sequences, a codon optimized nucleic acid sequence that encodes TPP1, and expression control sequences that direct expression of the encoded proteins in a host cell. In other embodiments, the rAAV expression cassette, the virus, the vector (such as rAAV vector), and/or the production plasmid further comprise one or more of an intron, a Kozak sequence, a polyA, post-transcriptional regulatory elements and others. In one embodiment, the post-transcriptional regulatory element is Woodchuck Hepatitis Virus (WHP) Posttranscriptional Regulatory Element (WPRE).

The expression cassettes, vectors and plasmids include other components that can be optimized for a specific species using techniques known in the art including, e.g, codon optimization, as described herein. The components of the cassettes, vectors, plasmids and viruses or other compositions described herein include a promoter sequence as part of the expression control sequences. In another embodiment, the promoter is cell-specific. The term “cell-specific” means that the particular promoter selected for the recombinant vector can direct expression of the optimized TPP1 coding sequence in a particular cell or tissue type. In one embodiment, the promoter is specific for expression of the transgene in ependyma, the epithelial lining of the brain ventricular system. In another embodiment, the promoter is specific for expression in a brain cell selected from neurons, astrocytes, oligoedendrocytes, and microglia. In one embodiment, the promoter is modified to add one or more restriction sites to facilitate cloning.

In another embodiment, the promoter is a ubiquitous or consistutive promoter. An example of a suitable promoter is a hybrid chicken β-actin (CBA) promoter with cytomegalovirus (CMV) enhancer elements, such as the sequence shown in SEQ ID NO: 5 at nt 3396 to 4061. In another embodiment, the promoter is the CB7 promoter. Other suitable promoters include the human β-actin promoter, the human elongation factor-1a promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLoS ONE 9(8): e106472. Still other suitable promoters include viral promoters, constitutive promoters, regulatable promoters [see, e.g., WO 2011/126808 and WO 2013/04943]. Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

In a further embodiment, the promoter is selected from SV40 promoter, the dihydrofolate reductase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In another embodiment, the promoter is an inducible promoter. The inducible promoter may be selected from known promoters including the rapamycin/rapalog promoter, the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24; 5:17105 and Daber R, Lewis M., A novel molecular switch. J Mol Biol. 2009 Aug. 28; 391(4):661-70, Epub 2009 Jun. 21 which are both incorporated herein by reference in their entirety.

In other embodiments, the expression cassette, vector, plasmid and virus described herein contain other appropriate transcription initiation, termination, enhancer sequences, efficient RNA processing signals such as splicing and polyadenylation (polyA) signals; TATA sequences; sequences that stabilize cytoplasmic mRNA; sequences that enhance translation efficiency (i.e., Kozak consensus sequence); introns; sequences that enhance protein stability; and when desired, sequences that enhance secretion of the encoded product. The expression cassette or vector may contain none, one or more of any of the elements described herein.

Examples of suitable polyA sequences include, e.g., a synthetic polyA or from bovine growth hormone (bGH), human growth hormone (hGH), SV40, rabbit β-globin (RGB), or modified RGB (mRGB). In a further embodiment, the poly A has a nucleic acid sequence from nt 33 to 159 of SEQ ID NO: 5.

Examples of suitable enhancers include, e.g., the CMV enhancer, the RSV enhancer, the alpha fetoprotein enhancer, the TTR minimal promoter/enhancer, LSP (TH-binding globulin promoter/alpha1-microglobulin/bikunin enhancer), an APB enhancer, ABPS enhancer, an alpha mic/bik enhancer, TTR enhancer, en34, ApoE amongst others.

In one embodiment, a Kozak sequence is included upstream of the TPP1 coding sequence to enhance translation from the correct initiation codon. In another embodiment, CBA exon 1 and intron are included in the expression cassette. In one embodiment, the TPP1 coding sequence is placed under the control of a hybrid chicken actin (CBA) promoter. This promoter consists of the cytomegalovirus (CMV) immediate early enhancer, the proximal chicken β actin promoter, and CBA exon 1 flanked by intron 1 sequences.

In another embodiment, the intron is selected from CBA, human beta globin, IVS2, SV40, bGH, alpha-globulin, beta-globulin, collagen, ovalbumin, p53, or a fragment thereof.

In one embodiment, the expression cassette, the vector, the plasmid and the virus contain a 5′ ITR, chicken beta-actin (CBA) promoter, CMV enhancer, CBA exon 1 and intron, human codon optimized CLN2 sequence, rabbit globin poly A and 3′ ITR. In a further embodiment, the expression cassette includes nt 1 to 4020 of SEQ ID NO: 8. In yet a further embodiment, the 5′ ITR has a nucleic acid sequence from nt 3199 to nt 3328 of SEQ ID NO: 5 and the 3′ITR has a nucleic acid sequence from nt 248 to nt 377 of SEQ ID NO: 5. In a further embodiment, the production plasmid has a sequence of SEQ ID NO: 5, also shown in FIGS. 1C-1E.

### II. Method of Treating Batten Disease and Pharmaceutical Compositions

### II-1. Method of Treating Batten Disease

In another aspect, a method for treating Batten disease caused by a defect in the CLN2 gene comprises delivering to a subject in need thereof a vector (such as rAAV) which encodes TPP1, as described herein. In one embodiment, a method of treating a subject having Batten disease with a rAAV described herein is provided. Also provided herein are methods of treating Batten disease comprising administering to a subject in need thereof the rAAV described herein via more than one route.

Provided herein in one aspect, is a method of treating CLN2 Batten disease in a subject comprising administering to a subject in need thereof a recombinant adeno-associated virus (rAAV) via a first route and a second route, and said first route and said second route are into the central nervous system (CNS), and said first route is into the brain region and said second route is into the spinal cord region, and said recombinant adeno-associated virus (rAAV) comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR.

In certain embodiments, the brain region may be the intrathecal space covering the brain. In certain embodiments, the brain region may be the cerebral ventricles. In certain embodiments, the brain region may be the cisterna magna. In certain embodiments, delivery into the brain region may be delivering into the cerebrospinal fluid (CSF).

In certain embodiments, the spinal cord region may be the intrathecal space around the spinal cord. In certain embodiments, the spinal cord region may be the spinal canal. In certain embodiments, the spinal cord region may be the subarachnoid space. In certain embodiments, delivery into the spinal cord region may be delivering into the cerebrospinal fluid (CSF).

In certain embodiments, said first route is intracerebroventricular (ICV) or intracisternal (IC). In other embodiments, said first route is an administration route into the brain region that is other than intracerebroventricular (ICV) or intracisternal (IC).

In certain embodiments, said second route is intrathecal-lumbar (IT-L). In other embodiments, said first route is an administration route into the spinal cord region that is other than intrathecal-lumbar (IT-L).

In certain embodiments, said method further comprises administering to said subject said rAAV via a third route, wherein said third route is selected from the group consisting of intracerebroventricular (ICV), intracisternal (IC), intrathecal-lumbar, intracranial, intravenous, intravascular, intraarterial, intramuscular, intraocular, subcutaneous, and intradermal. In certain embodiments, said third route delivers the rAAV to the liver. In a specific embodiment, said third route is intravenous.

In certain embodiments, the method of treating CLN2 Batten disease in a subject comprises co-administering to a subject in need thereof said rAAV via intracerebroventricular (ICV) and intrathecal-lumbar (IT-L) routes. In certain embodiments, the method of treating CLN2 Batten disease in a subject comprises co-administering to a subject in need thereof said rAAV via intracisternal (IC) and intrathecal-lumbar (IT-L) routes. In certain embodiments, the method of treating CLN2 Batten disease in a subject comprises co-administering to a subject in need thereof said rAAV via intracerebroventricular (ICV), intrathecal-lumbar (IT-L), and intravenous routes. In certain embodiments, the method of treating CLN2 Batten disease in a subject comprises co-administering to a subject in need thereof said rAAV via intracisternal (IC), intrathecal-lumbar (IT-L), and intravenous routes.

In another aspect, provided is a method of treating CLN2 Batten disease in a subject comprising administering to a subject in need thereof a recombinant adeno-associated virus (rAAV) via a first route and a second route, and said first route is into the central nervous system (CNS), and said second route delivers the rAAV outside of the CNS, and said recombinant adeno-associated virus (rAAV) comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, said first route is intrathecal-lumbar (IT-L), intracerebroventricular (ICV) or intracisternal (IC). In certain embodiments, said second route is selected from the group consisting of intravenous, intravascular, intraarterial, intramuscular, intraocular, subcutaneous, and intradermal. In a specific embodiment, said second route is intravenous.

In another aspect, provided is a method of treating CLN2 Batten disease in a subject comprising administering to a subject in need thereof a recombinant adeno-associated virus (rAAV) via a first route and a second route, and said first route is into the central nervous system (CNS), and said second route delivers the rAAV to the liver, and said recombinant adeno-associated virus (rAAV) comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR.

In certain embodiments, said first route is intrathecal-lumbar (IT-L), intracerebroventricular (ICV) or intracisternal (IC). In other embodiments, said first route is an administration route into the CNS that is other than intrathecal-lumbar (IT-L), intracerebroventricular (ICV) or intracisternal (IC).

In certain embodiments, said second route is selected from the group consisting of intravenous, intravascular, intraarterial, intramuscular, intraocular, subcutaneous, and intradermal. In a specific embodiment, said second route is intravenous. In other embodiments, said second route is an administration route delivering the rAAV to the liver that is other than intravenous, intravascular, intraarterial, intramuscular, intraocular, subcutaneous, and intradermal.

In certain embodiments, the method of treating CLN2 Batten disease in a subject comprises co-administering to a subject in need thereof said rAAV into the CNS and intravenous route. In certain embodiments, the method of treating CLN2 Batten disease in a subject comprises co-administering to a subject in need thereof said rAAV via intrathecal and intravenous routes. In certain embodiments, the method of treating CLN2 Batten disease in a subject comprises co-administering to a subject in need thereof said rAAV via intrathecal-lumbar (IT-L) and intravenous routes. In certain embodiments, the method of treating CLN2 Batten disease in a subject comprises co-administering to a subject in need thereof said rAAV via intracerebroventricular (ICV) and intravenous routes. In certain embodiments, the method of treating CLN2 Batten disease in a subject comprises co-administering to a subject in need thereof said rAAV via intracisternal (IC) and intravenous routes. In certain embodiments, the method of treating CLN2 Batten disease in a subject comprises co-administering to a subject in need thereof said rAAV via a route into the CNS, which is other than intrathecal-lumbar (IT-L), intracerebroventricular (ICV) or intracisternal (IC), and intravenous routes.

In certain embodiments, the method of treating CLN2 Batten disease in a subject comprises co-administering to a subject in need thereof said rAAV via a route into the CNS and intravascular route. In certain embodiments, the method of treating CLN2 Batten disease in a subject comprises co-administering to a subject in need thereof said rAAV via intrathecal and intravascular routes. In certain embodiments, the method of treating CLN2 Batten disease in a subject comprises co-administering to a subject in need thereof said rAAV via intrathecal-lumbar (IT-L) and intravascular routes. In certain embodiments, the method of treating CLN2 Batten disease in a subject comprises co-administering to a subject in need thereof said rAAV via intracerebroventricular (ICV) and intravascular routes. In certain embodiments, the method of treating CLN2 Batten disease in a subject comprises co-administering to a subject in need thereof said rAAV via intracisternal (IC) and intravascular routes. In certain embodiments, the method of treating CLN2 Batten disease in a subject comprises co-administering to a subject in need thereof said rAAV via a route into the CNS, which is other than intrathecal-lumbar (IT-L), intracerebroventricular (ICV) or intracisternal (IC), and intravascular routes.

In certain embodiments, the method of treating CLN2 Batten disease in a subject comprises co-administering to a subject in need thereof said rAAV via a route into the CNS and intraarterial route. In certain embodiments, the method of treating CLN2 Batten disease in a subject comprises co-administering to a subject in need thereof said rAAV via intrathecal and intraarterial routes. In certain embodiments, the method of treating CLN2 Batten disease in a subject comprises co-administering to a subject in need thereof said rAAV via intrathecal-lumbar (IT-L) and intraarterial routes. In certain embodiments, the method of treating CLN2 Batten disease in a subject comprises co-administering to a subject in need thereof said rAAV via intracerebroventricular (ICV) and intraarterial routes. In certain embodiments, the method of treating CLN2 Batten disease in a subject comprises co-administering to a subject in need thereof said rAAV via intracisternal (IC) and intraarterial routes. In certain embodiments, the method of treating CLN2 Batten disease in a subject comprises co-administering to a subject in need thereof said rAAV via a route into the CNS, which is other than intrathecal-lumbar (IT-L), intracerebroventricular (ICV) or intracisternal (IC), and intraarterial routes.

In certain embodiments, the method of treating CLN2 Batten disease in a subject comprises co-administering to a subject in need thereof said rAAV via a route into the CNS and intramuscular route. In certain embodiments, the method of treating CLN2 Batten disease in a subject comprises co-administering to a subject in need thereof said rAAV via intrathecal and intramuscular routes. In certain embodiments, the method of treating CLN2 Batten disease in a subject comprises co-administering to a subject in need thereof said rAAV via intrathecal-lumbar (IT-L) and intramuscular routes. In certain embodiments, the method of treating CLN2 Batten disease in a subject comprises co-administering to a subject in need thereof said rAAV via intracerebroventricular (ICV) and intramuscular routes. In certain embodiments, the method of treating CLN2 Batten disease in a subject comprises co-administering to a subject in need thereof said rAAV via intracisternal (IC) and intramuscular routes. In certain embodiments, the method of treating CLN2 Batten disease in a subject comprises co-administering to a subject in need thereof said rAAV via a route into the CNS, which is other than intrathecal-lumbar (IT-L), intracerebroventricular (ICV) or intracisternal (IC), and intramuscular routes.

In certain embodiments, the method of treating CLN2 Batten disease in a subject comprises co-administering to a subject in need thereof said rAAV via a route into the CNS and intraocular route. In certain embodiments, the method of treating CLN2 Batten disease in a subject comprises co-administering to a subject in need thereof said rAAV via intrathecal and intraocular routes. In certain embodiments, the method of treating CLN2 Batten disease in a subject comprises co-administering to a subject in need thereof said rAAV via intrathecal-lumbar (IT-L) and intraocular routes. In certain embodiments, the method of treating CLN2 Batten disease in a subject comprises co-administering to a subject in need thereof said rAAV via intracerebroventricular (ICV) and intraocular routes. In certain embodiments, the method of treating CLN2 Batten disease in a subject comprises co-administering to a subject in need thereof said rAAV via intracisternal (IC) and intraocular routes. In certain embodiments, the method of treating CLN2 Batten disease in a subject comprises co-administering to a subject in need thereof said rAAV via a route into the CNS, which is other than intrathecal-lumbar (IT-L), intracerebroventricular (ICV) or intracisternal (IC), and intraocular routes.

In certain embodiments, the method of treating CLN2 Batten disease in a subject comprises co-administering to a subject in need thereof said rAAV via a route into the CNS and subcutaneous routes. In certain embodiments, the method of treating CLN2 Batten disease in a subject comprises co-administering to a subject in need thereof said rAAV via intrathecal and subcutaneous routes. In certain embodiments, the method of treating CLN2 Batten disease in a subject comprises co-administering to a subject in need thereof said rAAV via intrathecal-lumbar (IT-L) and subcutaneous routes. In certain embodiments, the method of treating CLN2 Batten disease in a subject comprises co-administering to a subject in need thereof said rAAV via intracerebroventricular (ICV) and subcutaneous routes. In certain embodiments, the method of treating CLN2 Batten disease in a subject comprises co-administering to a subject in need thereof said rAAV via intracisternal (IC) and subcutaneous routes. In certain embodiments, the method of treating CLN2 Batten disease in a subject comprises co-administering to a subject in need thereof said rAAV via a route into the CNS, which is other than intrathecal-lumbar (IT-L), intracerebroventricular (ICV) or intracisternal (IC), and subcutaneous routes.

In certain embodiments, the method of treating CLN2 Batten disease in a subject comprises co-administering to a subject in need thereof said rAAV via a route into the CNS and intradermal route. In certain embodiments, the method of treating CLN2 Batten disease in a subject comprises co-administering to a subject in need thereof said rAAV via intrathecal and intradermal routes. In certain embodiments, the method of treating CLN2 Batten disease in a subject comprises co-administering to a subject in need thereof said rAAV via intrathecal-lumbar (IT-L) and intradermal routes. In certain embodiments, the method of treating CLN2 Batten disease in a subject comprises co-administering to a subject in need thereof said rAAV via intracerebroventricular (ICV) and intradermal routes. In certain embodiments, the method of treating CLN2 Batten disease in a subject comprises co-administering to a subject in need thereof said rAAV via intracisternal (IC) and intradermal routes. In certain embodiments, the method of treating CLN2 Batten disease in a subject comprises co-administering to a subject in need thereof said rAAV via a route into the CNS, which is other than intrathecal-lumbar (IT-L), intracerebroventricular (ICV) or intracisternal (IC), and intradermal routes.

In certain embodiments, methods of treating CLN2 Batten disease provided herein may comprise administering said rAAV via said first route simultaneously with administering said rAAV via said second route.

In certain embodiments, methods of treating CLN2 Batten disease provided herein may comprise administering said rAAV via said first route prior to administering said rAAV via said second route. In certain embodiments, methods of treating CLN2 Batten disease provided herein may comprise administering said rAAV via said first route after administering said rAAV via said second route.

In certain embodiments, the interval between administration said rAAV via said first route and administering said rAAV via said second route may be about 0.5 hour, 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

In certain embodiments, the interval between administration said rAAV via said first route and administering said rAAV via said second route may be 0.5 hour, 1 hour, 2 hours, 3 hours, 4 hours, 5 hours, 6 hours, 7 hours, 8 hours, 9 hours, 10 hours, 11 hours, 12 hours, about 1 day, 2 days, 3 days, 4 days, 5 days, 6 days, 1 week, 8 days, 9 days, 10 days, 11 days, 12 days, 13 days, 2 weeks, 3 weeks, 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, or more.

In certain embodiments, the methods of treating CLN2 Batten disease provided herein may result in an increased TPP1 activity in the spinal cord of said subject. In certain embodiments, the methods of treating CLN2 Batten disease provided herein comprise may result in a TPP1 activity in the spinal cord of said subject that is at least 2%, 3%, 5%, 6%, 7%, 8%, 9%, 10%, 12%, 15%, 17%, 20%, 25%, 30%, 35%, 40%, 45%, or 50% higher than a reference TPP1 activity in the spinal cord of a second subject, and wherein the reference TPP1 activity in the spinal cord is measured when said second subject does not receive the treatment using said method, and wherein said second subject is the same or different from said subject. In certain embodiments, the methods of treating CLN2 Batten disease provided herein comprise may result in a TPP1 activity in the spinal cord of said subject that is 2%, 3%, 5%, 6%, 7%, 8%, 9%, 10%, 12%, 15%, 17%, 20%, 25%, 30%, 35%, 40%, 45%, or 50% higher than a reference TPP1 activity in the spinal cord of a second subject, and wherein the reference TPP1 activity in the spinal cord is measured when said second subject does not receive the treatment using said method, and wherein said second subject is the same or different from said subject.

In certain embodiments, the methods of treating CLN2 Batten disease provided herein may result in an increased hepatic TPP1 activity of said subject. In certain embodiments, the methods of treating CLN2 Batten disease provided herein comprise may result in a hepatic TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference hepatic TPP1 activity in a second subject, and wherein the reference hepatic TPP1 activity is measured when said second subject does not receive the treatment using said method, and wherein said second subject is the same or different from said subject. In certain embodiments, the methods of treating CLN2 Batten disease provided herein comprise may result in a hepatic TPP1 activity of said subject that is 2%, 3%, 5%, 6%, 7%, 8%, 9%, 10%, 12%, 15%, 17%, 20%, 25%, 30%, 35%, 40%, 45%, or 50% higher than a reference hepatic TPP1 activity in a second subject, and wherein the reference hepatic TPP1 activity is measured when said second subject does not receive the treatment using said method, and wherein said second subject is the same or different from said subject.

In certain embodiments, the methods of treating CLN2 Batten disease provided herein may result in an increased serum TPP1 activity of said subject. In certain embodiments, the methods of treating CLN2 Batten disease provided herein comprise may result in a serum TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference serum TPP1 activity in a second subject, and wherein the reference serum TPP1 activity is measured when said second subject does not receive the treatment using said method, and wherein said second subject is the same or different from said subject. In certain embodiments, the methods of treating CLN2 Batten disease provided herein comprise may result in a serum TPP1 activity of said subject that is 2%, 3%, 5%, 6%, 7%, 8%, 9%, 10%, 12%, 15%, 17%, 20%, 25%, 30%, 35%, 40%, 45%, or 50% higher than a reference serum TPP1 activity in a second subject, and wherein the reference serum TPP1 activity is measured when said second subject does not receive the treatment using said method, and wherein said second subject is the same or different from said subject.

In certain embodiments, the methods of treating CLN2 Batten disease provided herein may result in a reduced microglial activity in the cortex of said subject. In certain embodiments, the methods of treating CLN2 Batten disease provided herein comprise may result in a microglial activity in the cortex of said subject that is at least 2%, 3%, 5%, 6%, 7%, 8%, 9%, 10%, 12%, 15%, 17%, 20%, 25%, 30%, 35%, 40%, 45%, or 50% lower than a reference microglial activity in the cortex in a second subject, and wherein the reference microglial activity in the cortex is measured when said second subject does not receive the treatment using said method, and wherein said second subject is the same or different from said subject. In certain embodiments, the methods of treating CLN2 Batten disease provided herein comprise may result in a microglial activity in the cortex of said subject that is 2%, 3%, 5%, 6%, 7%, 8%, 9%, 10%, 12%, 15%, 17%, 20%, 25%, 30%, 35%, 40%, 45%, or 50% lower than a reference microglial activity in the cortex in a second subject, and wherein the reference microglial activity in the cortex is measured when said second subject does not receive the treatment using said method, and wherein said second subject is the same or different from said subject.

In certain embodiments, the methods of treating CLN2 Batten disease provided herein may result in an increase TPP1 activity in the brain of said subject. In certain embodiments, the methods of treating CLN2 Batten disease provided herein may result in a TPP1 activity in the brain of said subject that is at least 2%, 3%, 5%, 6%, 7%, 8%, 9%, 10%, 12%, 15%, 17%, 20%, 25%, 30%, 35%, 40%, 45%, or 50% higher than a reference TPP1 activity in the brain of a second subject, wherein the reference TPP1 activity in the brain is measured when said second subject does not receive the treatment using said method, and wherein said second subject is the same or different from said subject. In certain embodiments, the methods of treating CLN2 Batten disease provided herein may result in a TPP1 activity in the brain of said subject that is 2%, 3%, 5%, 6%, 7%, 8%, 9%, 10%, 12%, 15%, 17%, 20%, 25%, 30%, 35%, 40%, 45%, or 50% higher than a reference TPP1 activity in the brain of a second subject, wherein the reference TPP1 activity in the brain is measured when said second subject does not receive the treatment using said method, and wherein said second subject is the same or different from said subject.

In certain embodiments, said rAAV is administered in a therapeutically effective amount.

In certain embodiments, said subject is human.

In certain embodiments, the coding sequence of (c) is a codon optimized human CLN2, which is at least 70% identical to the native human coding sequence of SEQ ID NO: 2. In certain embodiments, the coding sequence of (c) is SEQ ID NO: 3.

In certain embodiments, the rAAV capsid is an AAV9 or a variant thereof.

In certain embodiments, the promoter is a chicken beta actin (CBA) promoter. In certain embodiments, the promoter is a hybrid promoter comprising a CBA promoter sequence and cytomegalovirus enhancer elements.

In certain embodiments, the AAV 5′ ITR and/or AAV3′ ITR is from AAV2.

In certain embodiments, the vector genome further comprises a polyA. In certain embodiments, the polyA is a synthetic polyA or from bovine growth hormone (bGH), human growth hormone (hGH), SV40, rabbit β-globin (RGB), or modified RGB (mRGB). In certain embodiments, the vector genome further comprises an intron. In certain embodiments, the intron is from CBA, human beta globin, IVS2, SV40, bGH, alpha-globulin, beta-globulin, collagen, ovalbumin, or p53.

In certain embodiments, the vector genome further comprises an enhancer. In certain embodiments, the enhancer is a CMV enhancer, an RSV enhancer, an APB enhancer, ABPS enhancer, an alpha mic/bik enhancer, TTR enhancer, en34, ApoE.

In certain embodiments, the vector genome is about 3 kilobases to about 5.5 kilobases in size. In certain embodiments, the vector genome is about 4 kilobases in size.

In certain embodiments, the rAAV is manufactured using a method comprising growing in suspension culture a suspension cell line that is capable of producing the rAAV. In certain embodiments, said suspension cell line is HEK293 suspension cell line.

In certain embodiments of this invention, a subject has neuronal ceroid lipofuscinosis (NCL), for which the components, compositions and methods of this invention are designed to treat. As used herein, the term “subject” as used herein means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

The neuronal ceroid-lipofuscinoses (NCLs) are a group of inherited, neurodegenerative, lysosomal storage disorders characterized by progressive intellectual and motor deterioration, seizures, and early death. Visual loss is a feature of most forms. Clinical phenotypes have been characterized traditionally according to the age of onset and order of appearance of clinical features into infantile, late-infantile, juvenile, adult, and Northern epilepsy (also known as progressive epilepsy with mental retardation [EPMR]). There is however genetic and allelic heterogeneity; a proposed new nomenclature and classification system has been developed to take into account both the responsible gene and the age at disease onset; for example, CLN2 disease, classic late infantile. The first symptoms typically appear between age two and four years, usually starting with epilepsy, followed by regression of developmental milestones, myoclonic ataxia, and pyramidal signs. Visual impairment typically appears at age four to six years and rapidly progresses to light/dark awareness only. Life expectancy ranges from age six years to early teenage. As used herein, the term “Batten disease” is used to refer to a CLN2 disease, which is used interchangeably with “NCL”.

As used herein, the term “treatment” or “treating” is defined encompassing administering to a subject one or more compounds or compositions described herein for the purposes of amelioration of one or more symptoms of Batten disease. “Treatment” can thus include one or more of reducing onset or progression of neuronal ceroid lipofuscinosis (NCL), preventing disease, reducing the severity of the disease symptoms, or retarding their progression, including the progression of blindness, removing the disease symptoms, delaying onset of disease or monitoring progression of disease or efficacy of therapy in a given subject.

By “administering” as used in the methods means delivering the composition to the target selected cell which is characterized by a defect in the CLN2 gene. In one embodiment, the method involves delivering the composition by intrathecal injection. In another embodiment, ICV injection to the subject is employed. In another embodiment, intrathecal-lumbar (IT-L) injection to the subject is employed. In one embodiment, the method involves delivering the composition via intracisternal (IC) injection (i.e., intrathecal delivery via image-guided suboccipital puncture into the cisterna magna). As used herein, the term intrathecal may, in some embodiments, refer to intraci sternal injection. In still another method, intravascular injections may be employed. In another embodiment, intramuscular injection is employed. Still other methods of administration may be selected by one of skill in the art given this disclosure.

By “administering” or “route of administration” is delivery of composition described herein, with or without a pharmaceutical carrier or excipient, of the subject. Routes of administration may be combined, if desired. In some embodiments, the administration is repeated periodically. The pharmaceutical compositions described herein are designed for delivery to subjects in need thereof by any suitable route or a combination of different routes. In some embodiments, direct delivery to the brain (optionally via intrathecal, intracisternal, ICV or IT-L injection), or delivery via systemic routes is employed, e.g., intravascular, intraarterial, intraocular, intravenous, intramuscular, subcutaneous, intradermal, and other parental routes of administration. The nucleic acid molecules, the expression cassette and/or vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus), alone or in combination with proteins. As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube. A device which is useful for delivering the compositions described herein into cerebrospinal fluid is described in PCT/US2017/16133, which is incorporated herein by reference.

### II-2. Pharmaceutical Compositions

In another aspect, also provided herein are pharmaceutical compositions.

In certain embodiments, the pharmaceutical compositions provided herein comprises (a) a recombinant adeno-associated virus (rAAV), (b) sodium chloride, (c) magnesium chloride, (d) potassium chloride, (e) dextrose, (f) poloxamer 188, (g) sodium phosphate monobasic, and (h) sodium phosphate dibasic. In certain embodiments, the pharmaceutical composition further comprises calcium chloride.

In certain embodiments, the rAAV in the pharmaceutical composition can be any rAAV that is known in the art. In certain embodiments, the rAAV in the pharmaceutical composition is any rAAV that is disclosed in the following patent applications, PCT/US2017/027650 (published as International Publication No.: WO 2017/181021), PCT/US2018/027568 (published as International Publication No.: WO 2018/191666), PCT/US2018/015910 (published as International Publication No.: WO 2018/144441), PCT/US2018/052855 (published as International Publication No.: WO 2019/067540), PCT/US2019/042205, PCT/US2019/043631, WO 2019079494 A1, WO 2019164854 A1, WO 2019079496 A2, US 20190211091A1, US 2019038777 A1, US 2018289839 A1, US 2019127455 A1, KR 20160010526 A, KR 20190086503 A, TW 201903146 A, WO 2019204514 A1, WO 2019204514 A1, WO 2019191114 A1, WO 2019169004 A1, WO 2019168961 A1, WO 2019164854 A1, WO 2019113224 A1, WO 2019108856 A1, WO 2019108857 A1, WO 2019060662 A1, WO 2019035066 A1, WO 2019036484 A1, WO 2019010335 A1, WO 2018232149 A1, WO 2018218359 A1, WO 2018209205 A1, WO 2018204626 A1, WO 2018200542 A1, WO 2018200419 A1, WO 2018191490 A1, WO 2018183293 A1, WO 2018160849 A1, WO 2018160582 A8, WO 2018160573 A1, WO 2018160585 A2, WO 2018152485 A1, WO 2018144709 A2, WO 2018126112 A1, WO 2018126116 A1, WO 2018059549 A1, WO 2018057916 A1, WO 2018022905 A2, WO 2018022511 A1, WO 2018009814 A1, WO 2017196814 A1, WO 2017184463 A1, WO 2017181068 A1, WO 2017180936 A1, WO 2017180854 A1, WO 2017180857 A1, WO 2017151884 A1, WO 2017151823 A1, WO 2017147180 A1, WO 2017136500 A1, WO 2017136533 A1, WO 2017120294 A1, WO 2017114497 A1, WO 2017106345 A1, WO 2017106354 A1, WO 2017106326 A1, WO 2017106244 A1, WO 2017106202 A2, WO 2017100676 A1, WO 2017100704 A1, WO 2017100674 A1, WO 2017100682 A1, WO 2017160360 A9, WO 2017087900 A1, WO 2017079656 A2, WO 2017062750 A1, WO 2017053732 A2, WO 2017040524 A1, WO 2017040528 A1, WO 2017024198 A1, WO 2017015102 A1, WO 2016196328 A1, WO 2016200543 A8, WO 2016179034 A2, WO 2016176191 A1, WO 2016176212 A1, WO 2016073556 A1, WO 2016019364 A1, WO 2015175639 A1, WO 2015164723 A1, WO 2015138870 A2, WO 2015138357 A2, WO 2015066627 A1, WO 2015009575 A1, WO 2015012924 A2, WO 2014151341 A1, WO 2014151265 A1, WO 2014124282 A1, WO 2014059068 A1, WO 2014052693 A2, WO 2014012025 A2, WO 2013173702 A2, WO 2013162748 A1, WO 2013142337 A1, WO 2014011210 A1, WO 2013049493 A1, WO 2012158757 A1, WO 2012145572 A1, WO 2012112832 A1, WO 2012071318 A2, WO 2011126808 A9, WO 2011112554 A1, WO 2011060233 A1, WO 2011041502 A1, WO 2011038187 A1, WO 2011038063 A1, WO 2010138675 A1, WO 2010127097 A1, WO 2010102140 A1, WO 2010056759 A1, WO 2010051367 A1, WO 2010062562 A1, WO 2010040135 A1, WO 2010011642 A2, WO 2010008782 A1, WO 2009134681 A2, WO 2009136977 A2, WO 2009105084 A2, WO 2009073104 A2, WO 2009073103 A2, WO 2008150459 A1, WO 2008140812 A2, WO 2008085486 A1, WO 2008079172 A2, WO 2008019131 A2, WO 2008013928 A2, WO 2007130455 A2, WO 2007127264 A2, WO 2008027084 A2, WO 2007106476 A2, WO 2007070705 A2, WO 2007024708 A2, WO 2007002285 A2, WO 2006110689 A2, WO 2006102072 A2, WO 2006039218 A2, WO 2006078279 A2, WO 2005118611 A2, WO 2005062957 A2, WO 2005033321 A2, WO 2005030292 A2, WO 2005027995 A2, WO 2005018431 A2, WO 2005001103 A2, WO 2004108922 A3, WO 2004094606 A2, WO 2004009769 A2, WO 03093460 A1, WO 03057171 A2, WO 03046124 A2, WO 03052051 A3, WO 03052052 A3, WO 03042397 A3, WO 03038062 A2, WO 03024502 A2, WO 03014367 A1, WO 03000851 A2, WO 02100317 A2, WO 02082904 A2, WO 0230410 A2, WO 0220718 A2, WO 0210410 A1, WO 0183692 A2, WO 0174163 A1, WO 0172329 A1, WO 0123001 A2, WO 0123597 A9, WO 0057837 A2, WO 0055342 A1, WO 0028061 A2, WO 9944645 A1, WO 9943360 A1, WO 9931982 A1, WO 9915677 A1, WO 9914354 A1, WO 9915685 A1, WO 9910013 A1, WO 9639530 A3, WO 9639416 A1, WO 9626286 A1, and WO 9613598 A2 (all publications, patents and patent applications referred to herein are incorporated by reference in their entirety).

In certain embodiments, the rAAV in the pharmaceutical composition may be selected from the group consisting of RGX-121 (REGENXBIO Inc.), RGX-111 (REGENXBIO Inc.), RGX-314 (REGENXBIO Inc.), RGX-181 (REGENXBIO Inc.), RGX-501 (REGENXBIO Inc.), Glybera® (alipogene tiparvovec) (uniQure), Voretigene neparvovec (SPK-RPE65) (Spark Therapeutics; MieraGTx UK II Ltd/Syne Qua Non Ltd/UCL), rAAV2-CBSB-hRPE65 (UPenn; NEI), rAAV2-hRPE65 (HMO), SPK-CHM (Spark Therapeutics), CNGA3-ACHM (AGTC), CNGB3-ACHM (AGTC), scAAV2-P1ND4 (NEI), XLRS gene therapy (Biogen/AGTC), BMN-270 (Biomarin), SB-525 (Sangamo), DTX101 (Dimension Therapeutics), SPK-9001 (SPK-FIX) (Spark Therapeutics/Pfizer), AMT-060 (uniQure/St. Jude's Hospital), SB-FIX (Sangamo), scAAV2/8-LP1-hFIXco (St. Jude's Hospital/UCL), ADVM-043 (Adverum), AVXS-101 (AveXis), rAAVrh74.MCK. micro-Dystrophin (NICHD), LGMD2D (NCH), rAAV1.CMV. huFollistatin344 (NCH), rAAVrh74.MHCK7.DYSF.DV (NCH), ART-102 (Arthrogen), Intracerebral gene therapy (INSERM), CERE-110 (Ceregene), CERE-120 (Ceregene/Sangamo), AAV-hAADC (NIH), AAV2CUhCLN2 (Weill Cornell University; Abeona Therapeutics), SAF-301 (Lysogene), DTX301 (Dimension Therapeutics), and TT-034 (Tacere Therapeutics) (see Naso et al. BioDrugs. 2017; 31(4): 317-334).

In certain embodiments, the rAAV in the pharmaceutical compositions may comprise components from one or more adeno-associated virus serotypes selected from the group consisting of AAV1, AAV2, AAV2tYF, AAV3, AAV4, AAVS, AAV6, AAV7, AAV8, AAV9, AAV10, AAV11, and AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16. In some embodiments, the rAAV in the pharmaceutical composition comprises a capsid protein of the AAV8 or AAV9 serotype. In preferred embodiments, the rAAV in the pharmaceutical composition comprises components from AAV9.

In certain embodiments, the pharmaceutical composition comprise multiple compounds. In certain embodiments, the compounds are in different hydrate forms, for example the hydrate forms selected from the group consisting of but not limited to anhydrous, monohydrate, dihydrate, 3-hydrate, 4-hydrate, 5-hydrate, 6-hydrate, 7-hydrate, 8-hydrate, 9-hydrate, and 10-hydrate forms.

In certain embodiments, the weight/volume concentration of a compound in the pharmaceutical composition may be expressed based on the compound in anhydrous form having a molar amount that is equivalent to the compound in a different hydrate form. In certain embodiments, the anhydrous form may not exist in nature.

In certain embodiments, the compound in certain hydrate form in the pharmaceutical composition may represent the same compound in a different hydrate form that has the equivalent molar amount.

In certain embodiments, the pharmaceutical composition comprises calcium chloride, for example calcium chloride in dihydrate form. In other embodiments, the pharmaceutical composition does not contain calcium chloride.

In certain embodiments, the pH of the pharmaceutical composition is about 7.4. In certain embodiments, the pH of the pharmaceutical composition is about 6.0 to 8.8. In certain embodiments, the pH of the pharmaceutical composition is about 6.0 to 9.0. In certain embodiments, the pH of the pharmaceutical composition is about 6.0. In certain embodiments, the pH of the pharmaceutical composition is about 6.1. In certain embodiments, the pH of the pharmaceutical composition is about 6.2. In certain embodiments, the pH of the pharmaceutical composition is about 6.3. In certain embodiments, the pH of the pharmaceutical composition is about 6.4. In certain embodiments, the pH of the pharmaceutical composition is about 6.5. In certain embodiments, the pH of the pharmaceutical composition is about 6.6. In certain embodiments, the pH of the pharmaceutical composition is about 6.7. In certain embodiments, the pH of the pharmaceutical composition is about 6.8. In certain embodiments, the pH of the pharmaceutical composition is about 6.9. In certain embodiments, the pH of the pharmaceutical composition is about 7.0. In certain embodiments, the pH of the pharmaceutical composition is about 7.1. In certain embodiments, the pH of the pharmaceutical composition is about 7.2. In certain embodiments, the pH of the pharmaceutical composition is about 7.3. In certain embodiments, the pH of the pharmaceutical composition is about 7.4. In certain embodiments, the pH of the pharmaceutical composition is about 7.5. In certain embodiments, the pH of the pharmaceutical composition is about 7.6. In certain embodiments, the pH of the pharmaceutical composition is about 7.7. In certain embodiments, the pH of the pharmaceutical composition is about 7.8. In certain embodiments, the pH of the pharmaceutical composition is about 7.9. In certain embodiments, the pH of the pharmaceutical composition is about 8.0. In certain embodiments, the pH of the pharmaceutical composition is about 8.1. In certain embodiments, the pH of the pharmaceutical composition is about 8.2. In certain embodiments, the pH of the pharmaceutical composition is about 8.3. In certain embodiments, the pH of the pharmaceutical composition is about 8.4. In certain embodiments, the pH of the pharmaceutical composition is about 8.5. In certain embodiments, the pH of the pharmaceutical composition is about 8.6. In certain embodiments, the pH of the pharmaceutical composition is about 8.7. In certain embodiments, the pH of the pharmaceutical composition is about 8.8. In certain embodiments, the pH of the pharmaceutical composition is about 8.9. In certain embodiments, the pH of the pharmaceutical composition is about 9.0.

In certain embodiments, the pH of the pharmaceutical composition is 7.4. In certain embodiments, the pH of the pharmaceutical composition is 6.0 to 8.8. In certain embodiments, the pH of the pharmaceutical composition is 6.0 to 9.0. In certain embodiments, the pH of the pharmaceutical composition is 6.0. In certain embodiments, the pH of the pharmaceutical composition is 6.1. In certain embodiments, the pH of the pharmaceutical composition is 6.2. In certain embodiments, the pH of the pharmaceutical composition is 6.3. In certain embodiments, the pH of the pharmaceutical composition is 6.4. In certain embodiments, the pH of the pharmaceutical composition is 6.5. In certain embodiments, the pH of the pharmaceutical composition is 6.6. In certain embodiments, the pH of the pharmaceutical composition is 6.7. In certain embodiments, the pH of the pharmaceutical composition is 6.8. In certain embodiments, the pH of the pharmaceutical composition is 6.9. In certain embodiments, the pH of the pharmaceutical composition is 7.0. In certain embodiments, the pH of the pharmaceutical composition is 7.1. In certain embodiments, the pH of the pharmaceutical composition is 7.2. In certain embodiments, the pH of the pharmaceutical composition is 7.3. In certain embodiments, the pH of the pharmaceutical composition is 7.4. In certain embodiments, the pH of the pharmaceutical composition is 7.5. In certain embodiments, the pH of the pharmaceutical composition is 7.6. In certain embodiments, the pH of the pharmaceutical composition is 7.7. In certain embodiments, the pH of the pharmaceutical composition is 7.8. In certain embodiments, the pH of the pharmaceutical composition is 7.9. In certain embodiments, the pH of the pharmaceutical composition is 8.0. In certain embodiments, the pH of the pharmaceutical composition is 8.1. In certain embodiments, the pH of the pharmaceutical composition is 8.2. In certain embodiments, the pH of the pharmaceutical composition is 8.3. In certain embodiments, the pH of the pharmaceutical composition is 8.4. In certain embodiments, the pH of the pharmaceutical composition is 8.5. In certain embodiments, the pH of the pharmaceutical composition is 8.6. In certain embodiments, the pH of the pharmaceutical composition is 8.7. In certain embodiments, the pH of the pharmaceutical composition is 8.8. In certain embodiments, the pH of the pharmaceutical composition is 8.9. In certain embodiments, the pH of the pharmaceutical composition is 9.0.

In certain embodiments, the pharmaceutical composition provided herein comprises a recombinant adeno-associated virus (rAAV) and one or more compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and one compound selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and two compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and three compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and four compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and five compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and six compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and all seven compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, provided herein is a pharmaceutical composition comprising:

(a) a recombinant adeno-associated virus (rAAV),
(b) sodium chloride,
(c) magnesium chloride,
(d) potassium chloride,
(e) dextrose,
(f) poloxamer 188,
(g) sodium phosphate monobasic, and
(h) sodium phosphate dibasic,
wherein said recombinant adeno-associated virus (rAAV) comprises an AAV capsid and a vector genome packaged therein, and wherein said vector genome comprising: (i) an AAV 5′ inverted terminal repeat (ITR) sequence; (ii) a promoter; (iii) a CLN2 coding sequence encoding a human TPP1; and (iv) an AAV 3′ ITR.

In certain embodiments, the pharmaceutical composition further comprising calcium chloride.

In certain embodiments, said sodium chloride, said magnesium chloride, said potassium chloride, said dextorse, said poloxamer 188, said sodium phosphate monobasic, said sodium phosphate dibasic, and said calcium chloride are each in anhydrous, monohydrate, dihydrate, 3-hydrate, 4-hydrate, 5-hydrate, 6-hydrate, 7-hydrate, 8-hydrate, 9-hydrate, or 10-hydrate form.

In certain embodiments, the pharmaceutical composition comprises

(a) said rAAV,
(b) sodium chloride at a concentration of about 8.77 g/L,
(c) magnesium chloride 6-hydrate, at a concentration of about 0.244 g/L,
(d) potassium chloride at a concentration of about 0.224 g/L,
(e) calcium chloride dihydrate at a concentration of about 0.206 g/L,
(f) dextorse anhydrous at a concentration of about 0.793 g/L,
(g) poloxamer 188 at a concentration of about 0.001% (volume/volume),
(h) sodium phosphate monobasic monohydrate at a concentration of about 0.0278 g/L, and
(i) sodium phosphate dibasic anhydrous at a concentration of about 0.114 g/L.

In certain embodiments, the vector genome concentration (VGC) of the pharmaceutical composition is about 1×10¹¹ GC/mL, about 3×10¹¹ GC/mL, about 6×10¹¹ GC/mL, about 1×10¹² GC/mL, about 3×10¹² GC/mL, about 6×10¹² GC/mL, about 1×10¹³ GC/mL, about 2×10¹³ GC/mL, about 3×10¹³ GC/mL, about 4×10¹³ GC/mL, about 5×10¹³ GC/mL, about 6×10¹³ GC/mL, about 7×10¹³ GC/mL, about 8×10¹³ GC/mL, about 9×10¹³ GC/mL, or about 1×10¹⁴ GC/mL, about 3×10¹⁴ GC/mL, about 6×10¹⁴ GC/mL, or about 1×10¹⁵ GC/mL. In certain embodiments, the vector genome concentration (VGC) of the pharmaceutical composition is 1×10¹¹ GC/mL, 3×10¹¹ GC/mL, 6×10¹¹ GC/mL, 1×10¹² GC/mL, 3×10¹² GC/mL, 6×10¹² GC/mL, 1×10¹³ GC/mL, 2×10¹³ GC/mL, about 3×10¹³ GC/mL, 4×10¹³ GC/mL, 5×10¹³ GC/mL, 6×10¹³ GC/mL, 7×10¹³ GC/mL, 8×10¹³ GC/mL, 9×10¹³ GC/mL, or 1×10¹⁴ GC/mL, 3×10¹⁴ GC/mL, 6×10¹⁴ GC/mL, or 1×10¹⁵ GC/mL.

In certain embodiments, the pH of the pharmaceutical composition is in a range from about 6.0 to about 9.0. In certain embodiments, the pH of the pharmaceutical composition is about 7.4.

In certain embodiments, the rAAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant rAAV in a reference pharmaceutical composition. In certain embodiments, the stability of the recombinant AAV is determined by an assay or assays disclosed in Section IV and EXAMPLES.

In certain embodiments, the stability of said rAAV in the pharmaceutical composition is determined by

(a) the infectivity of rAAV,
(b) the levels of aggregation of rAAV, or
(c) the levels of free DNA released by the rAAV.

In certain embodiments, the pharmaceutical composition is a liquid composition. In certain embodiments, the pharmaceutical composition is a frozen composition. In certain embodiments, the pharmaceutical composition is a lyophilized composition or a reconstituted lyophilized composition.

In certain embodiments, the pharmaceutical composition has a property that is suitable for intracerebroventricular (ICV), intracisternal (IC), intrathecal-lumbar, intracranial, intravenous, intravascular, intraarterial, intramuscular, intraocular, intramuscular, subcutaneous, or intradermal administration.

In certain embodiments, the coding sequence of (iii) of the rAAV in the pharmaceutical composition is a codon optimized human CLN2, which is at least 70% identical to the native human coding sequence of SEQ ID NO: 2. In certain embodiments, the coding sequence of (iii) of the rAAV in the pharmaceutical composition is SEQ ID NO: 3.

In certain embodiments, the rAAV capsid of the rAAV in the pharmaceutical composition is an AAV9 or a variant thereof.

In certain embodiments, the promoter of the rAAV in the pharmaceutical composition is a chicken beta actin (CBA) promoter. In certain embodiments, the promoter of the rAAV in the pharmaceutical composition is a hybrid promoter comprising a CBA promoter sequence and cytomegalovirus enhancer elements.

In certain embodiments, the AAV 5′ ITR and/or AAV3′ ITR of the rAAV in the pharmaceutical composition is from AAV2.

In certain embodiments, the vector genome of the rAAV in the pharmaceutical composition further comprises a polyA. In certain embodiments, the polyA is a synthetic polyA or from bovine growth hormone (bGH), human growth hormone (hGH), SV40, rabbit 3-globin (RGB), or modified RGB (mRGB).

In certain embodiments, the vector genome of the rAAV in the pharmaceutical composition further comprises an intron. In certain embodiments, the intron is from CBA, human beta globin, IVS2, SV40, bGH, alpha-globulin, beta-globulin, collagen, ovalbumin, or p53.

In certain embodiments, the vector genome of the rAAV in the pharmaceutical composition further comprises an enhancer. In certain embodiments, the enhancer is a CMV enhancer, an RSV enhancer, an APB enhancer, ABPS enhancer, an alpha mic/bik enhancer, TTR enhancer, en34, ApoE.

In certain embodiments, the vector genome of the rAAV in the pharmaceutical composition is about 3 kilobases to about 5.5 kilobases in size. In certain embodiments, the vector genome of the rAAV in the pharmaceutical composition is about 4 kilobases in size.

In certain embodiments, the rAAV in the pharmaceutical composition is manufactured using a method comprising growing in suspension culture a suspension cell line that is capable of producing the rAAV.

In another aspect, provide herein is a method of treating CLN2 Batten disease in a subject comprising administering to said subject the pharmaceutical composition provided herein. In certain embodiments, said pharmaceutical composition is administered in a therapeutically effective amount. In certain embodiments, said subject is human.

In yet another aspect, provide herein is a kit comprising one or more containers and instructions for use, wherein the one or more containers comprise the pharmaceutical composition provided herein.

In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.0005% (weight/volume, 0.005 g/L) to 0.05% (weight/volume, 0.5 g/L. In certain embodiments, the pharmaceutical composition comprises poloxamer 188 at a concentration of 0.0001% (weight/volume, 0.001 g/L) to 0.01% (weight/volume, 0.1 g/L). In certain embodiments, the pharmaceutical composition comprises poloxamer 188 at a concentration of 0.0005% (weight/volume, 0.005 g/L) to 0.001% (weight/volume, 0.01 g/L). In certain embodiments, the pharmaceutical composition comprises poloxamer 188 at a concentration of 0.001% (weight/volume, 0.01 g/L) to 0.05% (weight/volume, 0.5 g/L). In certain embodiments, the pharmaceutical composition comprises poloxamer 188 at a concentration of 0.0005% (weight/volume, 0.005 g/L). In certain embodiments, the pharmaceutical composition comprises poloxamer 188 at a concentration of 0.0006% (weight/volume, 0.006 g/L). In certain embodiments, the pharmaceutical composition comprises poloxamer 188 at a concentration of 0.0007% (weight/volume, 0.007 g/L). In certain embodiments, the pharmaceutical composition comprises poloxamer 188 at a concentration of 0.0008% (weight/volume, 0.008 g/L). In certain embodiments, the pharmaceutical composition comprises poloxamer 188 at a concentration of 0.0009% (weight/volume, 0.009 g/L). In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.002% (weight/volume, 0.02 g/L). In certain embodiments, the pharmaceutical composition comprises poloxamer 188 at a concentration of 0.003% (weight/volume, 0.03 g/L). In certain embodiments, the pharmaceutical composition comprises poloxamer 188 at a concentration of 0.004% (weight/volume, 0.04 g/L). In certain embodiments, the pharmaceutical composition comprises poloxamer 188 at a concentration of 0.005% (weight/volume, 0.05 g/L). In certain embodiments, the pharmaceutical composition comprises poloxamer 188 at a concentration of 0.01% (weight/volume, 0.1 g/L). In certain embodiments, the pharmaceutical composition comprises poloxamer 188 at a concentration of 0.05% (weight/volume, 0.5 g/L).

As used herein and unless otherwise specified, the term “about” means within plus or minus 10% of a given value or range.

The pharmaceutical compositions described herein are designed for delivery to subjects in need thereof by any suitable route or a combination of different routes.

In yet other aspects, these nucleic acid sequences, vectors, expression cassettes and rAAV viral vectors are useful in a pharmaceutical composition, which also comprises a pharmaceutically acceptable carrier, excipient, buffer, diluent, surfactant, preservative and/or adjuvant, etc. Such pharmaceutical compositions are used to express the optimized TPP1 in the host cells through delivery by such recombinantly engineered AAVs or artificial AAVs.

To prepare these pharmaceutical compositions containing the nucleic acid sequences, vectors, expression cassettes and rAAV viral vectors, the sequences or vectors or viral vector is preferably assessed for contamination by conventional methods and then formulated into a pharmaceutical composition suitable for administration to the patient. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, surfactant, or excipient etc. For injection, the carrier will typically be a liquid. Exemplary physiologically acceptable carriers include sterile, pyrogen-free water and sterile, pyrogen-free, phosphate buffered saline. A variety of such known carriers are provided in US Patent Publication No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

In one exemplary specific embodiment, the composition of the carrier or excipient contains 180 mM NaCl, 10 mM NaPi, pH7.3 with 0.0001%-0.01% Pluronic F68 (PF68). The exact composition of the saline component of the buffer ranges from 160 mM to 180 mM NaCl. Optionally, a different pH buffer (potentially HEPES, sodium bicarbonate, TRIS) is used in place of the buffer specifically described. Still alternatively, a buffer containing 0.9% NaCl is useful.

As used herein, the term “dosage” can refer to the total dosage delivered to the subject in the course of treatment, or the amount delivered in a single unit (or multiple unit or split dosage) administration. The pharmaceutical virus compositions can be formulated in dosage units to contain an amount of replication-defective virus carrying the codon optimized nucleic acid sequences encoding TPP1 as described herein that is in the range of about 1.0×10⁹ GC to about 1.0×10¹⁶ GC per dose including all integers or fractional amounts within the range. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰ or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹²×10¹² 3×10¹² 4×10¹² 5×10¹² 6×10¹² 7×10¹² 8×10¹² or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10₁₄, 3×10¹⁴, 4×10¹⁵, 5×10¹⁶, 6×10¹⁷, 7×10¹⁸, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range. In another embodiment, the compositions are formulated to contain at least 7.5×10¹² GC (7.5×10⁹ GC/g brain mass) to 2.7×10¹⁵ GC (2.1×10¹² GC/g brain mass). It is known in the art that the mass of the average human brain is about 1,300 g to about 1,400 g. It is also contemplated that the compositions here are useful in children, which have a range of brain mass from about 1000 g to about 1300 g. All dosages may be measured by any known method, including as measured by oqPCR or digital droplet PCR (ddPCR) as described in, e.g., M. Lock et al, Hum Gene Ther Methods. 2014 April; 25(2):115-25. doi: 10.1089/hgtb.2013.131, which is incorporated herein by reference.

In one embodiment, an aqueous suspension suitable for administration to a Batten patient is provided. The suspension comprises an aqueous suspending liquid and about 7.5×10⁹ GC or viral particles to about 2.1×10¹² GC or viral particles per gram of brain of a recombinant adeno-associated virus (rAAV) described herein useful as a therapeutic for Batten disease.

It may also be desirable to administer multiple “booster” dosages of the pharmaceutical compositions of this invention. For example, depending upon the duration of the transgene within the CNS, one may deliver booster dosages at 6 month intervals, or yearly following the first administration. The fact that AAV-neutralizing antibodies were not generated by administration of the rAAV vector should allow additional booster administrations.

Such booster dosages and the need therefor can be monitored by the attending physicians, using, for example, the TPP1 activity, and neurocognitive tests described in the examples below. Other similar tests may be used to determine the status of the treated subject over time. Selection of the appropriate tests may be made by the attending physician. Still alternatively, the method of this invention may also involve injection of a larger volume of virus-containing solution in a single or multiple infection to allow TPP1 activity levels close to those found in normal subjects.

These above doses may be administered in a variety of volumes of carrier, excipient or buffer formulation, ranging from about 100 microliters to about 50 mL, including all numbers within the range, depending on the size of the patient, the viral titer used, the route of administration, and the desired effect of the method. In one embodiment, the volume is less than 10 mL. In one embodiment, the volume of carrier, excipient or buffer is at least about 500 μL. In one embodiment, the volume is about 750 μL. In another embodiment, the volume is about 1 mL. In another embodiment, the volume is about 2 mL. In another embodiment, the volume is about 3 mL. In another embodiment, the volume is about 4 mL. In another embodiment, the volume is about 5 mL. In another embodiment, the volume is about 6 mL. In another embodiment, the volume is about 7 mL. In another embodiment, the volume is about 8 mL. In another embodiment, the volume is about 9 mL. In another embodiment, the volume is about 10 mL. In another embodiment, the volume is about 11 mL. In another embodiment, the volume is about 12 mL. In another embodiment, the volume is about 13 mL. In another embodiment, the volume is about 14 mL. In another embodiment, the volume is about 15 mL. In another embodiment, the volume is about 16 mL. In another embodiment, the volume is about 17 mL. In another embodiment, the volume is about 18 mL. In another embodiment, the volume is about 19 mL. In another embodiment, the volume is about 20 mL. In another embodiment, the volume is about 21 mL. In another embodiment, the volume is about 22 mL. In another embodiment, the volume is about 23 mL. In another embodiment, the volume is about 24 mL. In another embodiment, the volume is about 25 mL or more. In one embodiment, the maximum injected volume is about 10% of total cerebrospinal fluid volume.

In one embodiment, the viral constructs may be delivered in doses of from at least 1×10⁹ to about least 1×10¹³ GCs in volumes of about 100 microliters mL to about 1 mL for small animal subjects, such as mice. For larger veterinary subjects, the larger human dosages and volumes stated above are useful. See, e.g., Diehl et al, J. Applied Toxicology, 21:15-23 (2001) for a discussion of good practices for administration of substances to various veterinary animals. This document is incorporated herein by reference.

It is desirable that the lowest effective concentration of virus or other delivery vehicle be utilized in order to reduce the risk of undesirable effects, such as toxicity. Still other dosages in these ranges may be selected by the attending physician, taking into account the physical state of the subject, preferably human, being treated, the age of the subject, and the degree to which the disorder, has developed.

Yet another aspect described herein is a method for treating, retarding or halting progression of Batten disease in a mammalian subject. In one embodiment, an rAAV carrying the CLN2 native, modified or codon optimized sequence, preferably suspended in a physiologically compatible carrier, diluent, excipient and/or adjuvant, may be administered to a desired subject including a human subject in a therapeutically effective amount. This method comprises administering to a subject in need thereof any of the nucleic acid sequences, expression cassettes, rAAV genomes, plasmids, vectors or rAAV vectors or compositions containing them. In one embodiment, the composition is delivered intrathecally. In another embodiment, the composition is delivered via ICV. In another embodiment, the composition is delivered intracisternally. In still another embodiment, the composition is delivered using a combination of administrative routes suitable for treatment of Batten disease, and may also involve intravenous administration or other conventional administration routes.

For use in these methods, the volume and viral titer of each dosage is determined individually, as further described herein. The dosages, administrations and regimens may be determined by the attending physician given the teachings of this specification. In another embodiment, the method involves administering the compositions in two or more dosages (e.g., split dosages). In another embodiment, a second administration of an rAAV including the selected expression cassette (e.g., CLN2 containing cassette) is performed at a later time point. Such time point may be weeks, months or years following the first administration. Such second administration is, in one embodiment, performed with an rAAV having a different capsid than the rAAV from the first administration. In another embodiment, the rAAV from the first and second administration have the same capsid.

In still other embodiments, the compositions described herein may be delivered in a single composition or multiple compositions. Optionally, two or more different AAV may be delivered, or multiple viruses (see, e.g., WO 2011/126808 and WO 2013/049493). In another embodiment, multiple viruses may contain different replication-defective viruses (e.g., AAV and adenovirus).

According to the present invention, a “therapeutically effective amount” of the hTPP1 is delivered as described herein to achieve a desired result, i.e., treatment of Batten disease or one or more symptoms thereof. A Unified Batten Disease Rating Scale (UBDRS) has been proposed which is a comprehensive system which physical, seizure, behavioral, and capability assessments. See, Mink, J., The Unified Batten Disease Rating Scale, accessible at rarediseases.info.nih.gov/files/mink.pdf. The CLN2 Disease Clinical Rating Scale (CRS) (i.e., Hamburg Scale developed by the University Medical Center Hamburg-Eppendorf, Germany) is one of the most commonly used tool to assess disease progression. The scale incorporates evaluation of disease progression in multiple functional areas, including loss of motor performance, seizure activity, loss of vision, and loss of language: each of the functional areas is rated such that the normal condition is assigned a score of ‘3’, a slight or just noticeable abnormality a score of ‘2’, and a severe abnormality a score of ‘1’, and a complete loss of function a score of 0. These scores are then summed to assign each patient a Total Disability Score Steinfeld et al, Late infantile neuronal ceroid lipofuscinosis: quantitative description of the clinical course in patients with CLN2 mutations, Am J Med Genet. 2002 Nov. 1; 112(4):347-54, which is incorporated herein by reference. See also, Wyrwich et al, An Adapted Clinical Measurement Tool for the Key Symptoms of CLN2 Disease, Journal of Inborn Errors of Metabolism & Screening, 2018, Volume 6: 1-7, which is incorporated herein by reference. The majority of CLN2 disease patients experience consistent and progressive loss of motor and language function without treatment, as measured using the CLN2 Disease CRS (Nickel et al., 2018). In one embodiment, the goal of treatment is to limit progression of the disease. This may be assessed by a quantitative and qualitative evaluation of symptoms, using the CLN2 disease rating disease scale or UBDRS.

In another embodiment, the method includes performing additional testing, e.g., assays and neurocognitive testing to determine the efficacy of the treatment. Such tests include those performed as part of the UBDRS, discussed above, and include, without limitation, assessment of: speech clarity, tongue protrusion, visual acuity, tone (arms, legs, neck), strength (arms, legs), hand tapping, heel stomping, spontaneous movements (akinesia), Stereotypies, Dystonia, myoclonus, tremor, chorea, dysmetria, gait, postural stability, seizures, behavior and mood, and overall health.

In one embodiment of the methods described herein, a one-time delivery of a composition as described herein, e.g., an AAV delivery of an optimized CLN2 cassette, is useful in treating Batten disease in a subject. In another embodiment of the methods described herein, a one-time delivery of a composition as described herein, e.g., an AAV delivery of an optimized CLN2 cassette, is useful in preventing Batten disease in a subject having a CLN2 defect.

Thus, in one embodiment, the composition is administered before disease onset. In another embodiment, the composition is administered prior to the initiation of neurological impairment. In another embodiment, the composition is administered after initiation of neurological impairment. In one embodiment, neonatal treatment is defined as being administered a TPP1 coding sequence, expression cassette or vector as described herein within 8 hours, the first 12 hours, the first 24 hours, or the first 48 hours of delivery. In another embodiment, particularly for a primate (human or non-human), neonatal delivery is within the period of about 12 hours to about 1 week, 2 weeks, 3 weeks, or about 1 month, or after about 24 hours to about 48 hours. In another embodiment, the composition is delivered after onset of symptoms. In one embodiment, treatment of the patient (e.g., a first injection) is initiated prior to the first year of life. In another embodiment, treatment is initiated after the first 1 year, or after the first 2 to 3 years of age, after 5 years of age, after 11 years of age, or at an older age. In one embodiment, treatment is initiated from ages about 4 years of age to about 12 years of age. In one embodiment, treatment is initiated on or after about 4 years of age. In one embodiment, treatment is initiated on or after about 5 years of age. In one embodiment, treatment is initiated on or after about 6 years of age. In one embodiment, treatment is initiated on or after about 7 years of age. In one embodiment, treatment is initiated on or after about 8 years of age. In one embodiment, treatment is initiated on or after about 9 years of age. In one embodiment, treatment is initiated on or after about 10 years of age. In one embodiment, treatment is initiated on or after about 11 years of age. In one embodiment, treatment is initiated on or after about 12 years of age. However, treatment can be initiated on or after about 15, about 20, about 25, about 30, about 35, or about 40 years of age. In one embodiment, treatment in utero is defined as administering the composition as described herein in the fetus. See, e.g., David et al, Recombinant adeno-associated virus-mediated in utero gene transfer gives therapeutic transgene expression in the sheep, Hum Gene Ther. 2011 April; 22(4):419-26. doi: 10.1089/hum.2010.007. Epub 2011 Feb. 2, which is incorporated herein by reference.

In another embodiment, the composition is readministered at a later date. Optionally, more than one readministration is permitted. Such readministration may be with the same type of vector, a different viral vector, or via non-viral delivery as described herein.

The goals of the treatments described herein include limiting or halting the progression of Batten disease. Desirable results of the treatments include, without limitation, increases in any of the assessment scores of the UBDRS and/or CLN2 Disease Rating Scale, an increase in TPP1 activity or expression levels, increase in (or reduction in progression of impairment of) motor function, as determined by neurocognitive testing, and increase in (or reduction in progression of impairment of) cortical volume by MRI. A desired result includes reducing muscle weakness, increasing muscle strength and tone, or maintaining or increasing respiratory health, or reducing tremors or twitching. Other desired endpoints can be determined by a physician.

In yet another embodiment, any of the above described methods is performed in combination with another, or secondary, therapy. The secondary therapy may be any now known, or as yet unknown, therapy which helps prevent, arrest or ameliorate these mutations or defects or any of the effects associated therewith. The secondary therapy can be administered before, concurrent with, or after administration of the compositions described above. In one embodiment, a secondary therapy involves non-specific approaches for maintaining the health of the retinal cells, such as administration of neurotrophic factors, anti-oxidants, anti-apoptotic agents. The non-specific approaches are achieved through injection of proteins, recombinant DNA, recombinant viral vectors, stem cells, fetal tissue, or genetically modified cells. The latter could include genetically modified cells that are encapsulated. In one embodiment, the secondary therapy is intracerebroventricular cerliponase alpha (BMN 190). See, Schulz et al, Intracerebroventricular cerliponase alfa (BMN 190) in children with CLN 2 disease: results from a phase 1/2 open label, dose-escalation study, J Inherit Metab Disease, 39:S51, which is incorporated herein by reference. The recommended dosage is 30-300 mg ICV infusion administered every other week.

In one embodiment, a method of generating a recombinant rAAV comprises obtaining a plasmid containing an AAV expression cassette as described above and culturing a packaging cell carrying the plasmid in the presence of sufficient viral sequences to permit packaging of the AAV viral genome into an infectious AAV envelope or capsid. Specific methods of rAAV vector generation are described above and may be employed in generating a rAAV vector that can deliver the codon optimized CLN2 in the expression cassettes and genomes described above and in the examples below.

In certain embodiments of this invention, a subject has Batten disease, for which the components, compositions and methods of this invention are designed to treat. As used herein, the term “subject” as used herein means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

As used herein, the term “treatment” or “treating” is defined encompassing administering to a subject one or more compounds or compositions described herein for the purposes of amelioration of one or more symptoms of Batten disease. “Treatment” can thus include one or more of reducing onset or progression of Batten disease, preventing disease, reducing the severity of the disease symptoms, or retarding their progression, including the progression of neurological impairment, removing the disease symptoms, delaying onset of disease or monitoring progression of disease or efficacy of therapy in a given subject.

In one aspect, a coding sequence is provided which encodes a functional TPP1 protein. By “functional hTPP1”, is meant a gene which encodes an TPP1 protein which provides at least about 50%, at least about 75%, at least about 80%, at least about 90%, or about the same, or greater than 100% of the biological activity level of the native TPP1 protein, or a natural variant or polymorph thereof which is not associated with disease.

A variety of assays exist for measuring TPP1 expression and activity levels in vitro. See, e.g., Example 2 below. The methods described herein can also be combined with any other therapy for treatment of Batten disease or the symptoms thereof. The management of CLN2 disease is complex. Patients require extensive multidisciplinary medical care due to the high symptom load and the rapid rate of functional decline, and families require extensive psychosocial support, yet no management guidelines currently exist for this condition. See, e.g., Williams et al, Management strategies for CLN2 disease, Pediatric Neurology 69 (2017) 102e112, which is incorporated herein by reference. However, in certain embodiments, the standard of care may include intracerebroventricular cerliponase alpha (BMN 190). See, Schulz et al, Intracerebroventricular cerliponase alfa (BMN 190) in children with CLN 2 disease: results from a phase ½ open label, dose-escalation study, J Inherit Metab Disease, 39:S51, which is incorporated herein by reference. The recommended dosage is 30-300 mg ICV infusion administered every other week.

In certain embodiments, the AAV9.CLN2 vector is produced. A number of suitable purification methods may be selected. Examples of suitable purification methods are described, e.g., International Patent Application No. PCT/US2016/065970, filed Dec. 9, 2016 and its priority documents, US Patent Application Nos. 62/322,071, filed Apr. 13, 2016 and 62/226,357, filed Dec. 11, 2015 and entitled “Scalable Purification Method for AAV9”, which is incorporated by reference herein.

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate contaminating host DNA from the production process. The DNase resistant particles are then subjected to heat treatment to release the genome from the capsid. The released genomes are then quantitated by real-time PCR using primer/probe sets targeting specific region of the viral genome (for example poly A signal). Another suitable method for determining genome copies are the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013]. Alternatively, ViroCyt3100 can be used for particle quantitation, or flow cytometry. In another method, the effective dose of a recombinant adeno-associated virus carrying a nucleic acid sequence encoding the optimized TPP1 coding sequence is measured as described in S. K. McLaughlin et al, 1988 J. Virol., 62:1963, which is incorporated by reference in its entirety.

The replication-defective virus compositions can be formulated in dosage units to contain an amount of replication-defective virus that is in the range of about 1.0×10⁹ GC to about 9×10¹⁵ GC (to treat an average subject of 70 kg in body weight) including all integers or fractional amounts within the range, and preferably 1.0×10¹² GC to 2.7×10¹⁵ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹² or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 2.7×10¹⁵ GC per dose including all integers or fractional amounts within the range.

In certain embodiments, the dose may be in the range of about 1×10⁹ GC/g brain mass to about 2.1×10¹² GC/g brain mass. In certain embodiments, the dose may be in the range of about 3×10¹⁰ GC/g brain mass to about 3×10¹¹ GC/g brain mass. In certain embodiments, the dose may be in the range of about 5×10¹⁰ GC/g brain mass to about 1.85×10¹¹ GC/g brain mass.

In one embodiment, the viral constructs may be delivered in doses of from at least about least 1×10⁹ GCs to about 2.1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. In one embodiment, the volume is about 10 mL or less. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. In still other embodiments, a patient may receive an intraci sternal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

The above-described recombinant vectors may be delivered to host cells according to published methods. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal or intracisternal delivery, a pH within this range may be desired; whereas for intravenous delivery, a pH of 6.8 to about 7.2 may be desired. In one embodiment, the pH is about 7.3. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 (BASF), also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate .7H2O), potassium chloride, calcium chloride (e.g., calcium chloride.2H2O), dibasic sodium phosphate, and mixtures thereof, in water. Suitably, for intrathecal or intracisternal delivery, the osmolarity is within a range compatible with cerebrospinal fluid (e.g., about 275 to about 290); see, e.g., emedicine.medscape.com/article/2093316-overview. Optionally, for intrathecal or intracisternal delivery, a commercially available diluent may be used as a suspending agent, or in combination with another suspending agent and other optional excipients. See, e.g., Elliotts B® solution (Lukare Medical). In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

In another embodiment, the composition includes a carrier, solvent, stabilizer, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

In one embodiment, the AAV9.CB7.hCLN2 drug product proposed configuration is a 1 mL frozen solution of AAV9.CB7.hCLN2 vector in formulation buffer contained in a 2 mL vial. The proposed formulation buffer is 150 mM sodium chloride, 1.2 mM magnesium chloride, 3 mM potassium chloride, 1.4 mM calcium chloride, 1 mM sodium phosphate, 4.4 mM dextrose, and 0.001% poloxamer 188, pH 7.3. The proposed quantitative composition of AAV9.CB7.hCLN2 drug product is provided in Table 1 below.

TABLE 1
Proposed Quantitative Composition of AAV9.CB7.hCLN2
Solution for Injection, 1 mL/Vial

| | Function | | | |
|---|---|---|---|---|
| Material | Active | Grade | Amount (per vial) | |
| AAV9.CB7.hCNLN2 | Substance | GMP | >1 × 10¹³ | GC/mL |
| Sodium Chloride | Stabiliser | USP/Ph. Eur./ | 8.76 | mg/mL |
| | | JP/BP/FCC | | |
| Magnesium Chloride | Stabiliser | USP/Ph. Eur./ | 0.11 | mg/mL |
| | | JP/BP/FCC | | |
| Potassium Chloride | Stabiliser | USP/Ph. Eur./ | 0.22 | mg/mL |
| | | JP/BP/FCC | | |
| Calcium Chloride | Stabiliser | USP/Ph. Eur./ | 0.16 | mg/mL |
| | | JP/BP/FCC | | |
| Sodium Phosphate | Stabiliser | USP/Ph. Eur./ | 0.16 | mg/mL |
| | | JP/BP/FCC | | |
| Dextrose | Stabiliser | USP/Ph. Eur./ | 0.79 | mg/mL |
| | | JP/BP/FCC | | |
| Poloxamer 188 | Surfactant | GMP | 0.001 | mL |
| Water for Injection | Solvent | USP/Ph. Eur. | q.s. to 1.0 | mL/vial |

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery. In one embodiment, intrathecal delivery encompasses an injection into the spinal canal, e.g., the subarachnoid space. In one embodiment, the route of delivery is intracerebroventricular injection (ICV). In another embodiment, the route of delivery is intrathecal-lumbar (IT-L) delivery. In yet another embodiment, the route of delivery is intracisternal (IC) injection (i.e., intrathecal delivery via image-guided suboccipital puncture into the cisterna magna).

The viral vectors described herein may be used in preparing a medicament for delivering hTPP1 to a subject (e.g., a human patient) in need thereof, supplying functional TPP1 to a subject, and/or for treating Batten disease. A course of treatment may optionally involve repeat administration of the same viral vector (e.g., an AAV9 vector) or a different viral vector (e.g., an AAV9 and an AAVrh10). Still other combinations may be selected using the viral vectors and non-viral delivery systems described herein.

The hTPP1 cDNA sequences described herein can be generated in vitro and synthetically, using techniques well known in the art. For example, the PCR-based accurate synthesis (PAS) of long DNA sequence method may be utilized, as described by Xiong et al, PCR-based accurate synthesis of long DNA sequences, Nature Protocols 1, 791-797 (2006). A method combining the dual asymmetrical PCR and overlap extension PCR methods is described by Young and Dong, Two-step total gene synthesis method, Nucleic Acids Res. 2004; 32(7): e59. See also, Gordeeva et al, J Microbiol Methods. Improved PCR-based gene synthesis method and its application to the Citrobacter freundii phytase gene codon modification. 2010 May; 81(2):147-52. Epub 2010 Mar. 10; see, also, the following patents on oligonucleotide synthesis and gene synthesis, Gene Seq. 2012 April; 6(1):10-21; U.S. Pat. Nos. 8,008,005; 7,985,565. Each of these documents is incorporated herein by reference. In addition, kits and protocols for generating DNA via PCR are available commercially. These include the use of polymerases including, without limitation, Taq polymerase; OneTaq® (New England Biolabs); Q5® High-Fidelity DNA Polymerase (New England Biolabs); and GoTaq® G2 Polymerase (Promega). DNA may also be generated from cells transfected with plasmids containing the hOTC sequences described herein. Kits and protocols are known and commercially available and include, without limitation, QIAGEN plasmid kits; Chargeswitch® Pro Filter Plasmid Kits (Invitrogen); and GenElute™ Plasmid Kits (Sigma Aldrich). Other techniques useful herein include sequence-specific isothermal amplification methods that eliminate the need for thermocycling. Instead of heat, these methods typically employ a strand-displacing DNA polymerase, like Bst DNA Polymerase, Large Fragment (New England Biolabs), to separate duplex DNA. DNA may also be generated from RNA molecules through amplification via the use of Reverse Transcriptases (RT), which are RNA-dependent DNA Polymerases. RTs polymerize a strand of DNA that is complimentary to the original RNA template and is referred to as cDNA. This cDNA can then be further amplified through PCR or isothermal methods as outlined above. Custom DNA can also be generated commercially from companies including, without limitation, GenScript; GENEWIZ®; GeneArt® (Life Technologies); and Integrated DNA Technologies.

The term “expression” is used herein in its broadest meaning and comprises the production of RNA or of RNA and protein. With respect to RNA, the term “expression” or “translation” relates in particular to the production of peptides or proteins. Expression may be transient or may be stable.

The term “translation” in the context of the present invention relates to a process at the ribosome, wherein an mRNA strand controls the assembly of an amino acid sequence to generate a protein or a peptide.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, “disease”, “disorder” and “condition” are used interchangeably, to indicate an abnormal state in a subject.

As used herein, the term “about” or “˜” means a variability of 10% from the reference given, unless otherwise specified.

The term “regulation” or variations thereof as used herein refers to the ability of a composition to inhibit one or more components of a biological pathway.

Unless defined otherwise in this specification, technical and scientific terms used herein have the same meaning as commonly understood by one of ordinary skill in the art and by reference to published texts, which provide one skilled in the art with a general guide to many of the terms used in the present application.

### III. Method of Manufacturing

Also provided herein are methods of manufacturing the rAAV described herein. In certain embodiments, the method comprises growing in suspension culture a suspension cell line that is capable of producing the rAAV.

In certain embodiments, buffers may be used in the method of manufacturing. Exemplary buffers include but are not limited to Tris, Bis-tris, Bis-tris propane, phosphate, and HEPES.

In certain embodiments, the time range for the conduct of the suspension main bioreactor process may be 1 to 10 days. In certain embodiments, the time range for the conduct of the suspension main bioreactor process may be 5 to 8 days. In certain embodiments, the time length for the conduct of the suspension main bioreactor process may be 1 day, 2 days, 3 days, 4 days, 5 days, 6 days, 7 days, 8 days, 9 days, or 10 days.

In certain embodiments, during the time range for the conduct of the suspension main bioreactor process, one or more of the pH, dissolved oxygen and temperature levels may be controlled. In certain embodiments, during the time range for the conduct of the suspension main bioreactor process, the pH level may be controlled. In certain embodiments, during the time range for the conduct of the suspension main bioreactor process, the dissolved oxygen level may be controlled. In certain embodiments, during the time range for the conduct of the temperature level may be controlled.

In certain embodiments, transient transfection may be carried out after 1 to 10 days of cell growth. In certain embodiments, transient transfection may be carried out after 1 day, 2 days, 3 days, 4 days, 5 days, 6 days, 7 days, 8 days, 9 days, or 10 days of cell growth. In certain embodiments, the cell growth may be carried out in a culture media comprising DMEM and 10% FBS.

In certain embodiments, transient transfection may be carried out using a mixture comprising pAAV.CB7.CI.CLN2.RBG.KanR vector genome plasmid, pAdDeltaF6, and pAAV29KanRGXRep2 AAV plasmid. In certain embodiments, the mixture comprises pAAV.CB7.CI.CLN2.RBG.KanR vector genome plasmid in an amount of 0.1 to 100 mg. In certain embodiments, the mixture comprises pAdDeltaF6 in an amount of 1 to 500 mg. In certain embodiments, the mixture comprises pAAV29KanRGXRep2 AAV plasmid in an amount of 1 to 500 mg. In certain embodiments, the cells may be incubated for 1 to 10 days after transient transfection. In certain embodiments, the cells may be incubated for 1 day, 2 days, 3 days, 4 days, 5 days, 6 days, 7 days, 8 days, 9 days, or 10 days after transient transfection.

In certain embodiments, during vector harvest, the cell culture may be supplemented with magnesium chloride in an amount of 0.1 to 10 mg. In certain embodiments, during vector harvest, the cell culture may be supplemented with magnesium chloride in an amount of 0.1 mg, 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg.

In certain embodiments, the purification process of the rAAV may comprise four steps, concentration and buffer exchange by TFF, affinity chromatography, ion exchange chromatography, and concentration and buffer exchange by TFF.

In certain embodiments, the buffer used in the concentration and buffer exchange by tangential flow filtration may comprise Tris, sodium chloride, and at a pH in a range of 6.0 to 8.5. Exemplary buffers include but are not limited to Tris, Bis-tris, Bis-tris propane, phosphate, and HEPES.

In certain embodiments, a buffer used in the affinity chromatography may comprise Tris, sodium chloride, and at a pH in a range of 6.0 to 8.5. In certain embodiments, a buffer used in the affinity chromatography may comprise Tris, sodium citrate, and at a pH in a range of 6.0 to 8.5. In certain embodiments, a buffer used in the affinity chromatography may comprise Bis-tris propane, pluronic F68, and at a pH in a range of 7 to 14. Exemplary buffers include but are not limited to Tris, Bis-tris, Bis-tris propane, phosphate, and HEPES.

In certain embodiments, the purification process of the rAAV comprises ion exchange chromatography. In certain embodiments, In certain embodiments, the purification process of the rAAV comprises cation exchange chromatography. In certain embodiments, the purification process of the rAAV comprises anion exchange chromatography. In certain embodiments, a buffer used in the ion exchange chromatography may comprise Bis Tris Propane, pluronic F68, and at a pH in a range of 4 to 14. Exemplary buffers include but are not limited to Tris, Bis-tris, Bis-tris propane, phosphate, and HEPES.

### IV. Assays

### IV-1. Assays Related to Method of Treating Batten Disease

The skilled artisan may use the assays as described herein and/or techniques known in the art (for example, assays described in WO2018209205A1) to study the composition and methods described herein, for example to study the rAAV provided herein in method of treating Batten disease. More details on the assays are provided in Examples 1 and 2. Examples 1 and 2 also demonstrate in more detail how such assays can be used to study the rAAV provided herein.

Related assays may include but are not limited to the following: in vivo study in TPP1^{mlJ} mice model for CLN2 Batten disease, natural history study of TPP1^{mlJ} knock out mice, pharmacology study in TPP1^{mlJ} KO Mice, assays measuring TPP1 enzyme activity, assessment of intracerebroventricular efficacy in mice using non-invasive full time monitoring in a digital vivarium, measuring effects of TPP1 replacement using AAV9 Delivery (ICV) in C57BL/6 TPP1^{mlJ} KO mice, safety pharmaceutical assays, toxicity study in mice, and pharmacodynamic studies in cynomolgus monkeys, assays for vector biodistribution, assays for vector shedding, repeat dose studies, carcinogenicity studies, and other toxicity studies.

### IV-2. Assays Related to Pharmaceutical Compositions

The skilled artisan may use the assays as described herein and/or techniques known in the art to study the composition and methods described herein, for example to test the formulations provided herein. More details on the assays are provided in Examples 3 and 4. Examples 3 and 4 also demonstrate in more detail how such assays can be used to test the formulations provided herein.

As described in Li et al., 2019 Cell & Gene Therapy Insights, 5(4):537-547 (incorporated by references herein in its entirety), exemplary assays include but are not limited the following: (1) Digital Droplet PCR (ddPCR) for Genome Copy Determinations; (2) Genome Content and % Full Capsid Analysis of AAV by Spectrophotometry; (3) Size Exclusion Chromatography to Determine DNA Distribution and Purity in Capsid; (4) Assessing Capsid Viral Protein Purity Using Capillary Electrophoresis; (5) In Vitro Potency Methods—Relative Infectivity as a Reliable Method for Quantifying Differences in the Infectivity of AAV Vectors in vitro; and (6) Analytical Ultracentrifugation (AUC) to Determine Capsid Empty/Full Ratios and Size Distributions.

### A. Freeze/Thaw Cycles Assay

Controlled freeze/thaw cycles can be run in the lyophilizer. Vials can be well-spaced on the shelves and 4 vials of buffer can be thermocoupled.

### B. Temperature Stress Assay

A temperature stress development stability study can be conducted at 1.0×10¹² GC/mL over 4 days at 37° C. to evaluate the relative stability of formulations provided herein.

Assays can be used to assess stability include but are not limited to in vitro relative potency (IVRP), vector genome concentration (VGC by ddPCR), free DNA by dye fluorescence, dynamic light scattering, appearance, and pH.

### C. Long-Term Stability Assay

Long-term development stability studies can be carried out for 12 months to demonstrate maintenance of in-vitro relative potency and other quality at −80° C. (≤−60° C.) and −20° C. (−25° C. to −15° C.) in the formulations provided herein.

### D. In Vitro Relative Potency (IVRP) Assay

To relate the ddPCR GC titer to gene expression, an in vitro bioassay may be performed by transducing HEK293 cells and assaying the cell culture supernatant for anti-VEGF Fab protein levels. HEK293 cells are plated onto three poly-D-lysine-coated 96-well tissue culture plates overnight. The cells are then pre-infected with wild-type human Ad5 virus followed by transduction with three independently prepared serial dilutions of Construct II reference standard and test article, with each preparation plated onto separate plates at different positions. On the third day following transduction, the cell culture media is collected from the plates and measured for VEGF-binding Fab protein levels via ELISA. For the ELISA, 96-well ELISA plates coated with VEGF are blocked and then incubated with the collected cell culture media to capture anti-VEGF Fab produced by HEK293 cells. Fab-specific anti-human IgG antibody is used to detect the VEGF-captured Fab protein. After washing, horseradish peroxidase (HRP) substrate solution is added, allowed to develop, stopped with stop buffer, and the plates are read in a plate reader. The absorbance or OD of the HRP product is plotted versus log dilution, and the relative potency of each test article is calculated relative to the reference standard on the same plate fitted with a four-parameter logistic regression model after passing the parallelism similarity test, using the formula: EC50 reference±EC50 test article. The potency of the test article is reported as a percentage of the reference standard potency, calculated from the weighted average of the three plates.

To relate the ddPCR GC titer to functional gene expression, an in vitro bioassay may be performed by transducing HEK293 cells and assaying for transgene (e.g. enzyme) activity. HEK293 cells are plated onto three 96-well tissue culture plates overnight. The cells are then pre-infected with wild-type human adenovirus serotype 5 virus followed by transduction with three independently prepared serial dilutions of enzyme reference standard and test article, with each preparation plated onto separate plates at different positions. On the second day following transduction, the cells are lysed, treated with low pH to activate the enzyme, and assayed for enzyme activity using a peptide substrate that yields increased fluorescence signal upon cleavage by transgene (enzyme). The fluorescence or RFU is plotted versus log dilution, and the relative potency of each test article is calculated relative to the reference standard on the same plate fitted with a four-parameter logistic regression model after passing the parallelism similarity test, using the formula: EC50 reference±EC50 test article. The potency of the test article is reported as a percentage of the reference standard potency, calculated from the weighted average of the three plates.

### E. Vector Genome Concentration Assay

Vector genome concentration GC can also be evaluated using ddPCR.

### F. Free DNA Analysis Using Dye Fluorescence Assay

Free DNA can be determined by fluorescence of SYBR® Gold nucleic acid gel stain (‘SYBR Gold dye’) that is bound to DNA. The fluorescence can be measured using a microplate reader and quantitated with a DNA standard. The results in ng/μL can be reported.

Two approaches can be used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1E6 is a unit conversion factor:

Total DNA(ng/μL)estimated=1E6×GC/mL(OD)×M(g/mol)/6.02E23

In the second approach, the sample can be heated to 85° C. for 20 min with 0.05% poloxamer 188 and the actual DNA measured in the heated sample by the SYBR Gold dye assay can be used as the total. This therefore has the assumption that all the DNA was recovered and quantitated. For example, the determination of total DNA by the SYBR gold dye (relative to the UV reading) can be found to be 131% for the Construct II dPBS formulation and 152% for the Construct II modified dPBS with sucrose formulation (This variation in the conversion of ng/μL to percentage of free DNA can be captured as a range in the reported results). For trending, either the raw ng/μL can be used or the percentage determined by a consistent method can be used.

### G. Size Exclusion Chromatography (SEC)

SEC can be performed using a Sepax SRT SEC-1000 Peek column (PN 215950P-4630, SN: 8A11982, LN: BT090, 5 μm 1000 A, 4.6×300 mm) on Waters Acquity Arc Equipment ID 0447 (C3PO), with a 25 mm pathlength flowcell. The mobile phase can be, for example, 20 mM sodium phosphate, 300 mM NaCl, 0.005% poloxamer 188, pH 6.5, with a flow rate of 0.35 mL/minute for 20 minutes, with the column at ambient temperature. Data collection can be performed with 2 point/second sampling rate and 1.2 nm resolution with 25 point mean smoothing at 214, 260, and 280 nm. The ideal target load can be 1.5E11 GC. The samples can be injected with 50 μL, about ⅓ of the ideal target or injected with 5 μL.

### H. Dynamic Light Scattering (DLS) Assay

Dynamic light scattering (DLS) can be performed on a Wyatt DynaProIII using Corning 3540 384 well plates with a 30 μL sample volume. Ten acquisitions each for 10 s can be collected per replicate and there were three replicate measurements per sample. The solvent can be set according to the solvent used in the samples, for example ‘PBS’ for Construct II in dPBS and ‘4% sucrose’ for the Construct II in modified dPBS with sucrose samples. Results not meeting data quality criteria (baseline, SOS, noise, fit) can be ‘marked’ and excluded from the analysis. The low delay time cutoff can be changed from 1.4 μs to 10 μs for the modified dPBS with sucrose samples to eliminate the impact of the sucrose excipient peak at about 1 nm on causing artifactually low cumulants analysis diameter results.

### I. Differential Scanning Calorimetry

Low temperature Differential Scanning calorimetry (low-temp DSC) can be run using a TA Instruments DSC250. About 20 μL of sample can be loaded into a Tzero pan and crimped with a Tzero Hermetic lid. Samples can be equilibrated at 25° C. for 2 min, then cooled at 5° C./min to −60° C., equilibrated for 2 min, then heated at 5° C./min to 25° C. Heat flow data can be collected in conventional mode.

### J. Real-Time Buffer pH Tracking

The pH of different formulation buffers was monitored with INLAB COOL PRO-ISM low temperature pH probe, which can detect pH down to −30° C. One milliliter buffer was placed in 15 mL Falcon tube and then the pH probe was submerged in the buffer. A piece of parafilm was used to seal the gap between Falcon tube and pH probe to avoid contamination and evaporation. The probe along with the Falcon tube was placed in −20 AD freezer. The pH and temperature of the buffer were recorded every 2.5 min for around 20 hour or until the pH versus temperature behavior achieved repeating pattern. The temperature change caused by the automatic defrosting process created a stress condition for buffer pH stability.

### K. Osmolality

The osmometer uses the technique of freezing-point depression to measure osmolality. Calibration of the instrument can be performed using 50 mOsm/kg, 850 mOsm/kg, and 2000 mOsm/kg NIST traceable standards. The reference solution of 290 mOsm/kg can be used to determine the system suitability of the osmometer.

### L. Density Measurement

The density can be measured with Anton Paar DMA500 densitometer, using water as reference. The densitometer can be washed with water and then methanol, followed by air-drying between samples.

### M. Viscosity Measurement

Viscosity can be measured using methods known in the art, for example methods provide in the United States Pharmacopeia (USP) published in 2019 and previous versions thereof (incorporated by reference herein in their entirety).

### N. Virus Infectivity Assay

TCID₅₀ infectious titer assay as described in Francois, et al. Molecular Therapy Methods & Clinical Development (2018) Vol. 10, pp. 223-236 (incorporated by reference herein in its entirety) can be used. Relative infectivity assay as described in Provisional Application 62/745,859 filed Oct. 15, 2018) can be used.

### O. Crystallization and Glass Transition Temperatures

Exemplary methods are described in Croyle et al., 2001, Gene Ther. 8(17):1281-90 (incorporated by reference in its entirety herein).

IV-3. Assays Related to Recombinant Adeno-associated Virus (rAAV) and Method of Manufacturing

The skilled artisan may use the assays as described herein and/or techniques known in the art (for example, assays described in WO2018209205A1) to study the composition and methods described herein, for example to study the rAAV provided herein. More details on the assays are provided in Example 5. Example 5 also demonstrates in more detail how such assays can be used to study the rAAV provided herein.

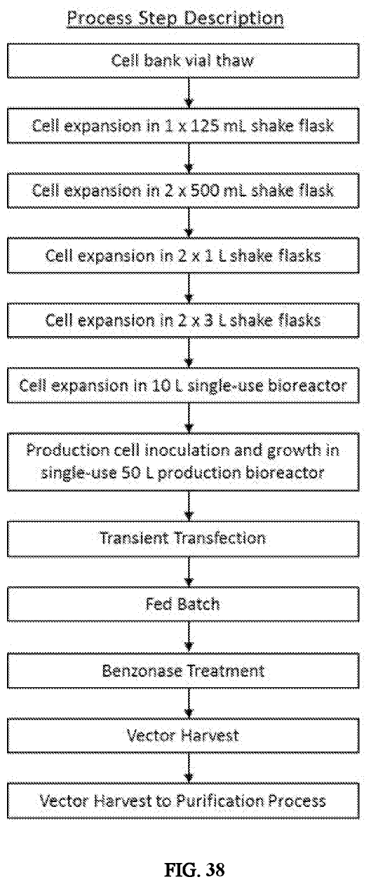

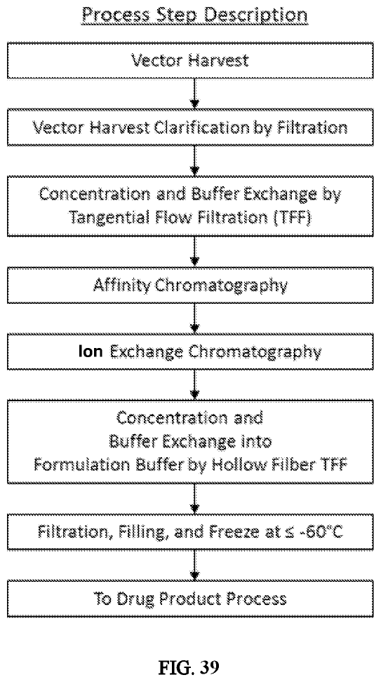

The manufacturing process for bulk drug substance is summarized in the flow diagrams presented in FIG. 38 and FIG. 39. The bulk drug substance can be manufactured by polyethylenimine (PEI)-mediated transient transfection of the HEK293 cells with the three plasmids described in Example 5.

Related assays may include but are not limited to the following: assays that involve in transient transfection, vector harvest, vector purification process, for example, concentration and buffer exchange by tangential flow filtration and affinity chromatography, ion exchange chromatography, and optional concentration by TFF or dilution.

### EXAMPLES

The following examples are illustrative only and are not intended to limit the present invention.

### Example 1: AAV9.CB7.hCLN2 Improves Survival and Neuropathology in TPP1^{mlJ} Mice, A Model For CLN2 Batten Disease

This example provides methods that may be used to evaluate the rAAV provided herein, for example AAV9.CB7.hCLN2, using TPP1^{mlJ} KO mice, a model of CLN2 disease.

TPP1^{mlJ} KO mice demonstrate characteristic features of CLN2 disease in humans, similar to alternative mouse models of CLN2 (Sleat et al, 2004. J. Neurosci; 24(41):9117-26; Geraets et al, 2017. PLoS One; 12(5):e0176526). The aim of this study was to establish the efficacy of AAV9.CB7.hCLN2 through assessment of lifespan, TPP1 activity and pathology in the brain of mice that lack the TPP1 enzyme.

Methods

Groups of TPP1^{mlJ} KO mice (n=10/sex/group; ˜4 weeks old) were administered a single intracerebroventricular (ICV) injection (5 μL) of AAV9.CB7.hCLN2 at doses of 0, 1.25×10¹⁰, 5×1010, 2×10¹¹ or 8.5×10¹¹ GC/animal. Animals were euthanized either 9 weeks after dosing (5/sex/group) or remained on study (5/sex/group) to evaluate the effect of AAV9.CB7.hCLN2 on lifespan. TPP1^{mlJ} KO mice are genotyped prior to dosing and at necropsy.

The following endpoints were evaluated: in-life observations, TPP1 activity (serum, brain [right hemisphere], spinal cord [thoracic] and liver), anti-TPP1 antibodies [serum] and neuropathology. Neuropathological examination evaluated neuronal loss in the brain, cervical and lumbar spinal cord, as well as intralysosomal accumulation of autofluorescent storage material, astrocytosis (GFAP) and microglial activation (CD68).

TPP1 enzymatic activity was assessed using an enzymatic assay that measures the cleavage of non-fluorescent AAF-AMC substrate to fluorescent free AMC by TPP1. The presence of anti-TPP1 antibodies were assessed by a solution bridging immunoassay using the MSD platform.

Results

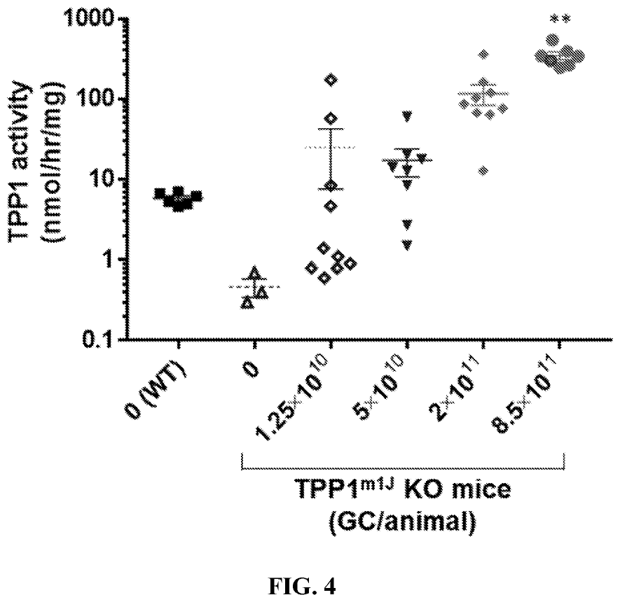

As shown in FIG. 2, survival increased in AAV9.CB7.hCLN2-treated TPP1^{mlJ} KO mice. AAV9.CB7.hCLN2 demonstrates an improvement in survival when administered to mice that lack the TPP1 enzyme at doses ≥2×10¹¹ GC/animal. Results showed 100% survival at the highest dose in both males and females.

Clinical signs observed in untreated TPP1^{mlJ} KO mice were generally seen from Week 8 onwards and included tremors and hind limbs splayed, typically associated with this disease model. In surviving AAV9.CB7.hCLN2-treated animals, clinical signs were comparable to those observed in untreated animals from Week 8 onwards (tremors and abnormal gait), however, food intake was normal and animals continued to increase body weight.

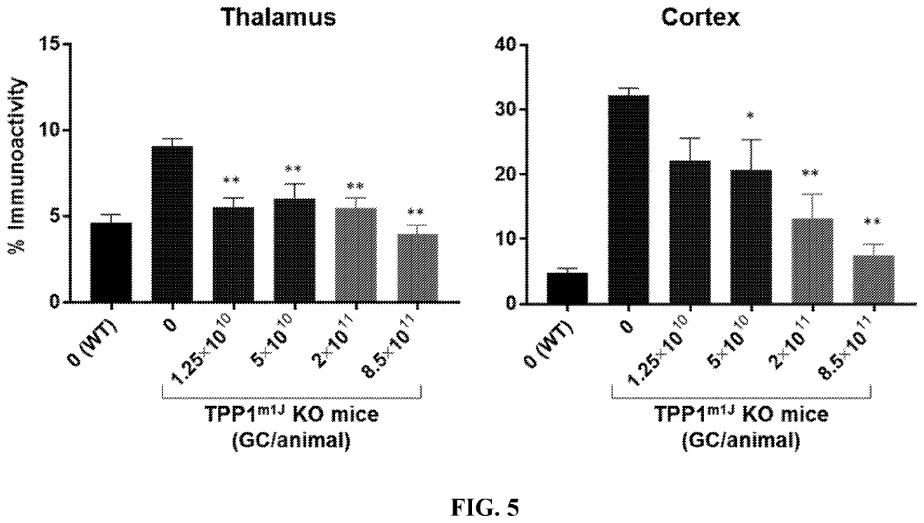

As shown in FIG. 3, TPP1 activity increased in the brain of AAV9.CB7.hCLN2-treated TPP1^{mlJ} KO mice. TPP1 activity was increased in all AAV9.CB7.hCLN2-treated animals with no sex-related differences. TPP1 activity was comparable at ≥5×10¹⁰ GC/animal.

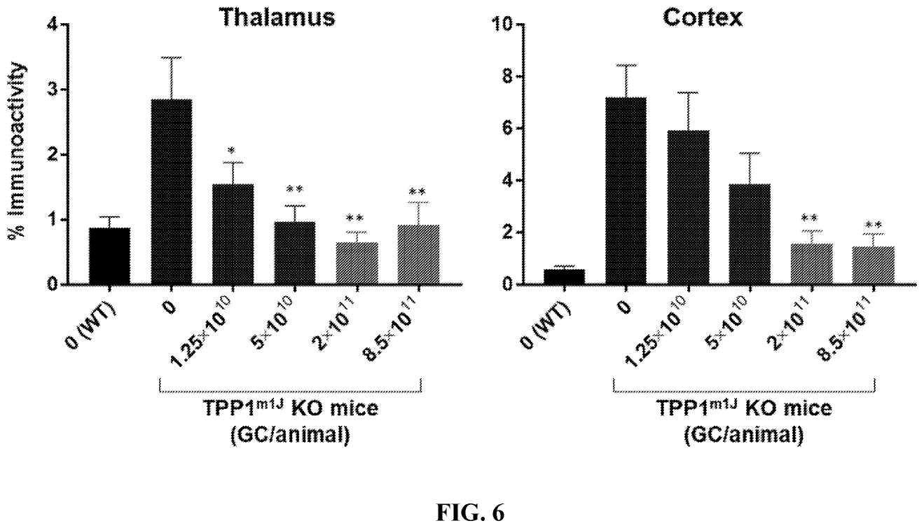

As shown in FIG. 4, TPP1 activity increased in the spinal cord of AAV9.CB7.hCLN2-treated TPP1^{mlJ} KO mice. Dose-related increases in TPP1 activity was observed in AAV9.CB7.hCLN2-treated animals.

At 9 weeks, there was a high incidence of anti-TPP1 antibodies, with the lowest signal observed at the high dose, which might be attributable to assay interference or reflect an induction of immune tolerance.

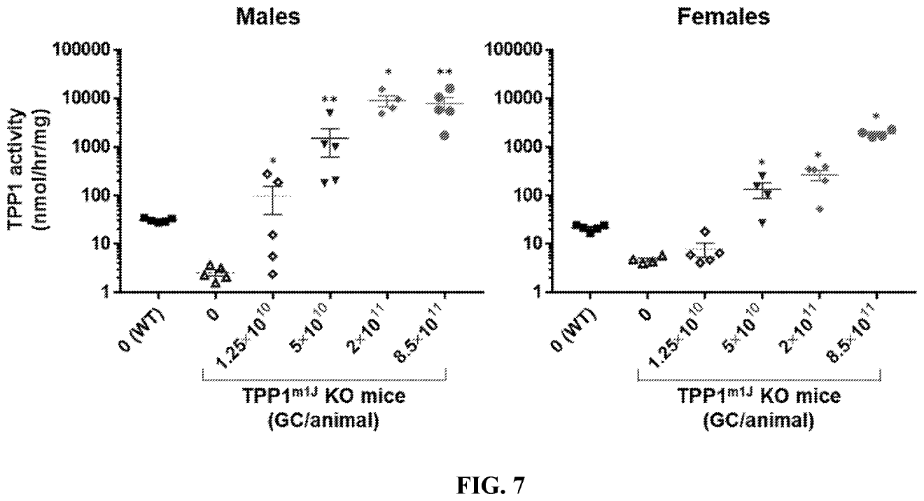

As shown in FIG. 5, astrocytosis decreased in AAV9.CB7.hCLN2-treated TPP1^{mlJ} KO mice. AAV9.CB7.hCLN2 decreased astrocytosis (GFAP) in the thalamus (VPM/VPL) and cortex (S1BF), with animals at the highest dose being comparable to WT animals.

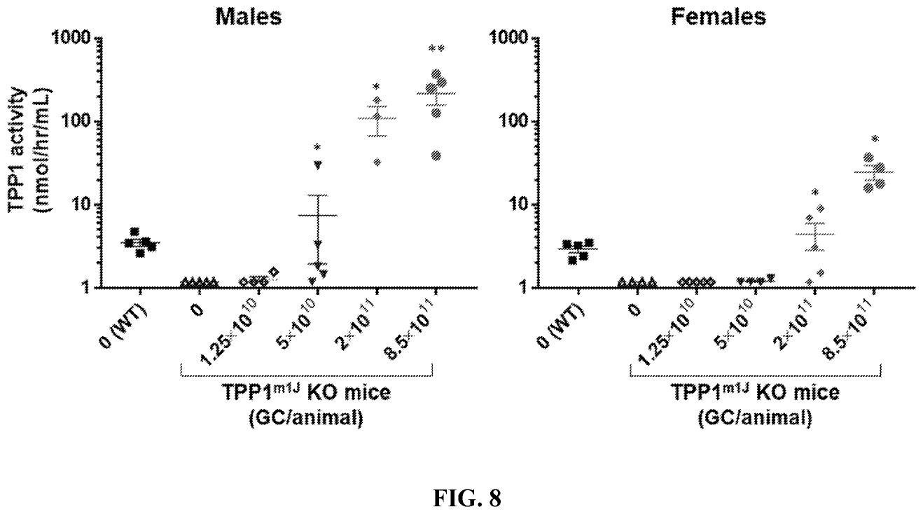

As shown in FIG. 6, microglian activation decreased in AAV9.CB7.hCLN2-treated TPP1^{mlJ} KO mice. AAV9.CB7.hCLN2 decreased microglial activation (CD68) in the thalamus (VPM/VPL) and cortex (S1BF). At ≥2×10¹¹ GC/animal, CD68 immunoreactivity was comparable to WT animals.

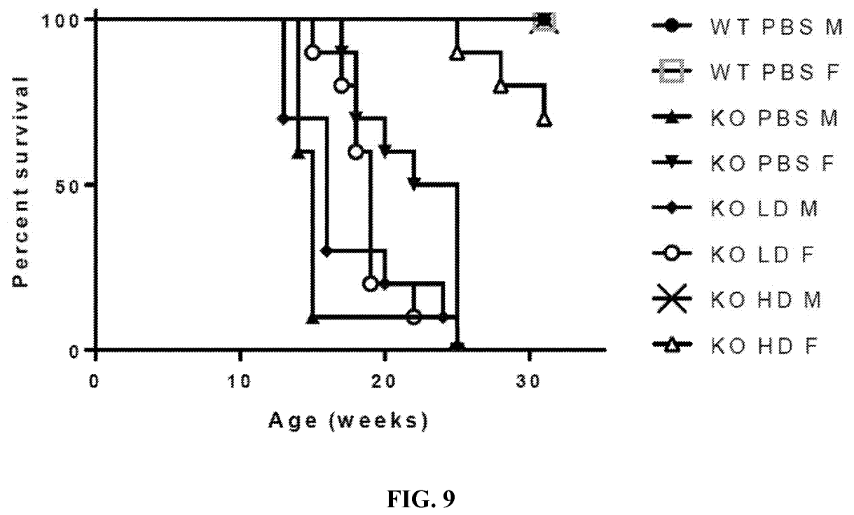

As shown in FIG. 7, hepatic TPP1 activity increased in AAV9.CB7.hCLN2-treated TPP1^{mlJ} KO mice. AAV9.CB7.CLN2 increased TPP1 activity in the liver, with greater increases seen in males.

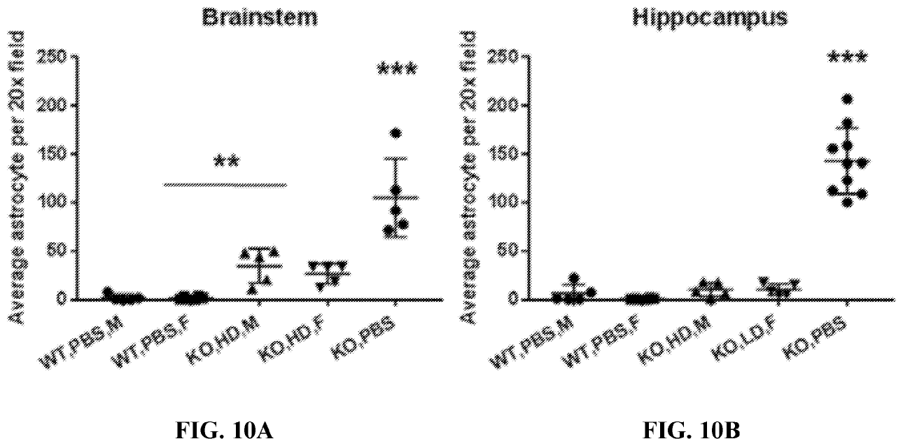

As shown in FIG. 8, serum TPP1 activity increased in AAV9.CB7.hCLN2-treated TPP1^{mlJ} KO mice. AAV9.CB7.hCLN2 increased TPP1 activity in the serum, with greater increases observed in males.

Conclusion

A single dose of AAV9.CB7.hCLN2 was shown to improve survival through increasing TPP1 activity in the brain, spinal cord and liver, decreasing astrocytosis and microglial activation in the thalamus and cortex in a biologically relevant animal model of CLN2 disease.

Increasing brain TPP1 activity alone did not improve survival indicating the potential therapeutic benefit of transducing the spinal cord and liver in this model.

The gender-related differences in transduction efficiency of the liver was considered a rodent specific androgen-dependent effect on the uptake of AAV, independent of serotype, promoter or transgene (Lonning et al, 2002. Molecular Therapy Vol. 5, No. 5; Davidoff et al, 2003. Blood. 15; 102(2):480-8). The minimum effective dose may be 2×10¹¹ GC/animal.

### Example 2: Studies on AAV9.CB7.hCLN2

This example provides methods that may be used to evaluate the rAAV provided herein, for example AAV9.CB7.hCLN2, including nonclinical studies and studies for effects in human.

### I. Nonclinical Studies

### A. Nonclinical Pharmacology

A series of in vivo nonclinical pharmacology studies were conducted with AAV9.CB7.hCLN2. These studies were conducted in a relevant mouse model of CLN2 disease and demonstrated an improvement in survival and increases in brain TPP1 activity following intracerebroventricular (ICV) administration of AAV9.CB7.hCLN2 into the CSF. Neuropathological analysis showed a reduction in neuronal loss in the brain, cervical and lumbar spinal cord as well as intralysosomal accumulation of autofluorescent storage material, astrocytosis and microglial activation.

The in vivo pharmacology studies were conducted using TPP10^{mlJ} knock out (KO) mice, a biologically relevant disease model for late-infantile neuronal ceroid lipofuscinosis (LINCL) Batten disease (CLN2). TPP1^{mlJ} KO mice have a single nucleotide mutation in the splice donor site downstream of exon 8 of the CLN2 gene, which encodes for the soluble lysosomal enzyme TPP1. This mutation resulted in lysosomal accumulation of lipofuscin, most prominently in neuronal tissue. Lipofuscin accumulation correlated with inflammation, activation of glial cells and subsequent neuronal degeneration. Neuronal degeneration occurred in the brain, spinal cord, and motor neurons. In a natural history study with TPP1^{mlJ} KO mice, these mice demonstrated characteristic features of CLN2 disease in humans, including early age at onset of clinical symptoms, rapid progression of the abnormal phenotype, and shortened lifespan, as well as similar pathophysiological, biochemical, and functional changes. The changes seen in TPP1^{mlJ} KO mice were similar to those seen in an alternative mouse models of CLN2, either the TPP1^{tmlPlob} or the Cln2^{R207X/R207X} mice (Sleat D E, et al. Am J Hum Genet. 1999; 64(6):1511-23; Geraets et al, 2017. PLoS One; 12(5):e0176526). Therefore, overall the TPP1^{mlJ} KO mice are considered a biologically relevant rodent model of CLN2 disease.

All murine studies were conducted by ICV injection of AAV9.CB7.hCLN2 into the CSF as administration into the cisterna magna is not possible in young mice. The biodistribution of AAV9 vector-based products following ICV injection is considered comparable to intrathecal injection into the cisterna magna (the proposed clinical route of administration). Nonclinical studies in multiple species have shown that biodistribution within the CNS and peripheral tissues of AAVs is comparable between these routes of administration (Haurigot V, et al. J Clin Invest. 2013; 123(8):3254-71; Hinderer et al, 2017; McLean et al, 2014, Belle et al, 2019). Therefore, this is an acceptable approach to evaluate the efficacy and safety of AAV9.CB7.hCLN2 in mice.

A summary of studies is listed in Table 2.

TABLE 2
Nonclinical Studies with AAV9.CB7.hCLN2

| | | | | | |
|---|---|---|---|---|---|
| | | | Doses | Dose | |
| Species | Study Type | ROA | (GC/animal) | (GC/g brain) | GLP Status |

Pharmacology

| | | | | | |
|---|---|---|---|---|---|
| TPP1^{m1J} KO mice | Natural history | NA | NA | NA | Non-GLP |
| | study | | | | |
| TPP1^{m1J} KO mice | In vivo efficacy | ICV | 3 × 10⁹ | 7.5 × 10⁹ | Non-GLP |
| | | | 3 × 10¹¹ | 7.5 × 10¹⁰ | |
| TPP1^{m1J} KO mice | In vivo efficacy | ICV | 3 × 10¹¹ | 7.5 × 10¹⁰ | Non-GLP |
| TPP1^{m1J} KO mice | In vivo efficacy | ICV | 1.25 × 10¹⁰ | 3.13 × 10⁹ | Non-GLP |
| | | | 5 × 10¹⁰ | 1.25 × 10¹⁰ | |
| | | | 2 × 10¹¹ | 5.00 × 10¹⁰ | |
| | | | 8.5 × 10¹¹ | 2.13 × 10¹¹ | |
| | | | (MFD) | (MFD) | |

Pharmacodynamics

| | | | | | |
|---|---|---|---|---|---|
| Cynomolgus | 4-week | CM | 3.4 × 10¹¹ | 5.26 × 10⁹ | Non-GLP |
| monkey | pharmacodynamic | CM | 3.2 × 10¹² | 4.59 × 10¹⁰ | |
| | study | CM | 2.9 × 10¹³ | 4.2 × 10¹¹ | |
| | | IT-L | 3.2 × 10¹² | 4.59 × 10¹⁰ | |

Toxicology

| | | | | | |
|---|---|---|---|---|---|
| C57B1/6 mice | 3-month | ICV | 1.25 × 10¹⁰ | 3.13 × 10⁹ | GLP |
| | toxicology | | 5 × 10¹⁰ | 1.25 × 10¹⁰ | |
| | | | 2 × 10¹¹ | 5.00 × 10¹⁰ | |
| | | | 8.5 × 10¹¹ | 2.13 × 10¹¹ | |
| | | | (MFD) | (MFD) | |
| Cynomolgus | 4-week | CM | 2.9 × 10¹³ | 4.2 × 10¹¹ | Non-GLP |
| monkey | investigative | | | | |
| | toxicity study | | | | |

GC: genome copies; GLP: Good Laboratory Practice; ICV: intracerebroventricular; CM: intrathecal cisterna magna; IT-L: intrathecal lumbar; KO: knock out; NA: not applicable; MFD: maximum feasible dose; ROA: route of administration; TPP1: tripeptidyl-peptidase-1.

### 1. In Vivo Pharmacology

### a) A Natural History Study of TPP1^{mlJ} Knock Out Mice

The purpose of this study was to characterize the phenotype and histopathology of TPP1^{mlJ} KO mice and the ability of the animal model to recapitulate key characteristics of CLN2 disease in humans. Five groups of 20 animals (10/sex/group) were included in this natural history study and were observed for up to 24 weeks. Groups of animals were also euthanized at 4 and 14 weeks of age to evaluate disease progression. Median survival was 16 weeks for male KO animals and 19.5 weeks for female KO animals and comparable to other mouse models of CLN2 disease (Sleat D E, et al. J Neurosci, 2004; 24(41):9117-26; Geraets et al, 2017. PLoS One; 12(5):e0176526). At both 1 and 3 months of age, TPP1^{mlJ} KO mice showed a lack of TPP1 enzymatic activity (undetectable levels) in the cerebrum and liver. The onset of the CLN2 clinical phenotype was approximately 3 months of age based on gait abnormality. The most notable disease symptom was sudden death after seizures at 3.5 months. Seizures were observed earlier for males than females and were associated with increased stress attributed to cage mate fights. Starting in the first weeks of life (and presumably before birth) of TPP1^{mlJ} KO mice, the deficiency in TPP1 enzymatic activity caused accumulation of lipid-containing residues of lysosomal digestion, also known as lipofuscin granules, in the cytoplasm of neurons. Lipofuscin accumulation in the cytoplasm of neurons was revealed by hematoxylin and eosin (H&E) staining in 1-month old TPP1^{mlJ} KO animals and correlated with an increase in astrocyte activation or astrocytosis (indicative of neuroinflammation). Subsequently, TPP1^{mlJ} KO mice experienced progressive deterioration of motor function and gait abnormalities, tremors, seizures, weight loss and inability to eat, and early mortality. Neuroinflammation identified by GFAP staining peaked at 3 months of age and correlated with disease onset in TPP1^{mlJ} KO animals. TPP1^{mlJ} KO animals surviving after disease onset showed progressive accumulation of storage material in neurons of the CNS.

These results demonstrate that TPP1^{mlJ} KO mice exhibit characteristic features of CLN2 disease in humans, including early age at onset of clinical symptoms, rapid progression of the abnormal phenotype, and shortened lifespan, as well as similar pathophysiological, biochemical, and functional changes. Based on this data, the TPP1^{mlJ} KO mice was considered a suitable model to evaluate the efficacy of AAV9.CB7.hCLN2.

b) Pharmacology of AAV9.CB7.hCLN2 in TPP1^{mlJ} KO Mice

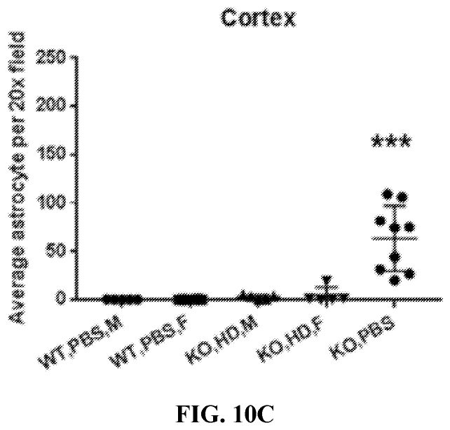

To test the therapeutic efficacy of AAV9.CB7.hCLN2 in TPP1^{mlJ} KO mice, AAV9.CB7.hCLN2 was administered ICV to 1-month old C57Bl/6 TPP1^{mlJ} KO mice (10/sex/group) at doses of 0 (PBS), 3×10⁹ GC/animal, or 3×10¹¹ GC/animal. Endpoints included clinical observations, motor coordination (rocking rotarod test), learning capacity (learning rotarod test), body weight, TPP1 activity, and anti-TPP1 antibodies. At study termination, 26 weeks post-ICV injection, TPP1 activity in brain and liver, vector liver distribution and correction of histopathology in the brain were assessed in surviving animals. There were no adverse clinical observations associated with AAV9.CB7.hCLN2 treatment. A dose-dependent increase in TPP1 levels that was associated with improvements in both CNS and peripheral parameters of CLN2-related endpoints was observed. At 3×10¹¹ GC/animal, survival in TPP1^{mlJ} KO mice was 100% for males and 70% for females after 26 weeks (FIG. 9).

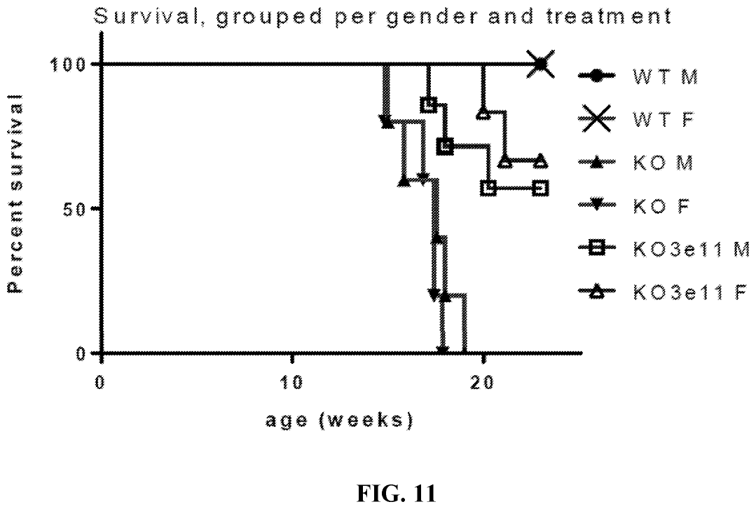

At 11 weeks after dosing, motor coordination (latency to fall from rocking rotarod) was comparable to WT animals TPP1^{mlJ} KO mice at 3×10¹¹GC/animal only; however, at 20 weeks after dosing, the latency to fall was reduced at this dose when compared with WT controls. Contrary to this, there were no differences between WT mice and TPP1^{mlJ} KO mice at 3×10¹¹GC/animal in motor learning at both 13 and 20 weeks after dosing. There were no improvements in motor coordination or learning in TPP1^{mlJ} KO mice at 3×10⁹GC/animal, which were comparable to untreated TPP1^{mlJ} KO mice. In TPP1^{mlJ} KO mice, accumulation of lysosomal storage in the cytoplasm of neurons throughout the brain was observed. In TPP1^{mlJ} KO mice at 3×10¹¹GC/animal, lysosomal storage material was reduced when compared with untreated TPP1^{mlJ} KO mice and neuronal cell morphology was considered normal. An evaluation of the brain for astrocytosis (GFAP staining) demonstrated that AAV9.CB7.hCLN2 can prevent astrocytosis with 100% efficiency in the cortex and hippocampus (FIG. 10). Decreases in astrocytosis was observed in the brainstem (FIG. 10). There was no impact on the survival or pathology 3×10⁹ GC/animal. Altogether, these results demonstrated the therapeutic efficacy of AAV9.CB7.hCLN2 to prevent clinical manifestation and histopathology of CLN2 disease.

TPP1 enzyme activity was measured in serum samples collected at 10 and 26 weeks post-injection. At 3×10¹¹GC/animal, animals expressed supra-physiologic TPP1 serum activity at 10 weeks post-injection and close to WT value 26 weeks after therapy. At 10 weeks, all AAV9.CB7.hCLN2-treated animals developed a high level of anti-TPP1 antibody in serum, particularly low dose treated animals. High dose treated males seemed to develop a milder humoral immune response to the transgene, possibly due to partial tolerization or interference with the assay at high concentrations of TPP1.

At study endpoint, TPP1 enzymatic activity was compared between PBS treated WT and high dose TPP1^{mlJ} KO mice in three organs: the cerebrum, the cerebellum/brainstem, and the liver. For each tissue, TPP1 activity was higher in treated TPP1^{mlJ} KO mice, demonstrating the high efficacy of AAV9.CB7.hCLN2 ICV therapy to restore TPP1 function. No gender-related differences were observed within the group with the exception of TPP1^{mlJ} KO mice, high dose liver TPP1 activity being 10-fold higher for male than for female.

In this study, the minimum effective dose was considered to be 3×10¹¹ GC/animal, which equates to 7.5×10¹¹ GC/g brain (estimated brain weight of 0.4 g), based on survival, in-life observations, reduction in lysosomal storage material, normal neuronal cell morphology, and prevention of astrocytosis. At this dose mean TPP1 activity was 19205 U/mg/h in the brain (combined cerebrum and cerebellum), 99991 U/mg/h in the liver, and 177 U/μL/h in serum.

c) An Assessment of Intracerebroventricular AAV9.CB7.hCLN2 Efficacy in Mice Using Non-Invasive Full Time Monitoring in a Digital Vivarium

To test the efficacy of AAV9.CB7.hCLN2 in TPP1^{mlJ} KO mice using unbiased non-invasive full-time monitoring in a digital vivarium (Vium, Inc., USA), AAV9.CB7.hCLN2 was administered ICV to 7 week-old TPP1^{mlJ} KO mice (7 males and 6 females) at 3×10¹¹ GC/animal. Treated animals were compared with age-matched vehicle-treated TPP1^{mlJ} KO (5 males and 5 females) and WT mice (5 males and 8 females). The following endpoints were continuously recorded, which included nightly motion, daily motion, breathing rate, and circadian rhythms. Recordings started 2 weeks after AAV9.CB7.hCLN2 ICV injection (age 9 weeks) and continued until the scheduled sacrifice (16 weeks post-injection, age 23 weeks) or earlier in cases of unscheduled death. At the end of the study, brain and liver weight as well as an assessment of histopathology in the brain and liver were conducted in surviving animals. Untreated TPP1^{mlJ} KO mice had tremors, hunched posture, abnormal gait, seizures and either euthanized in poor condition or found dead. Clinical signs were observed starting at 14.9 weeks; whereas, 54% of AAV9.CB7.hCLN2-treated TPP1^{mlJ} KO mice (7 out of 13) were clinically unremarkable throughout the study. The average age at the first clinical event was 16.1 weeks in TPP1^{mlJ} KO vehicle-treated mice and 18.6 weeks in the six AAV9.CB7.HCLN2-treated TPP1^{mlJ} KO mice; seven AAV9.CB7.hCLN2-treated TPP1^{mlJ} KO mice had no clinical signs.

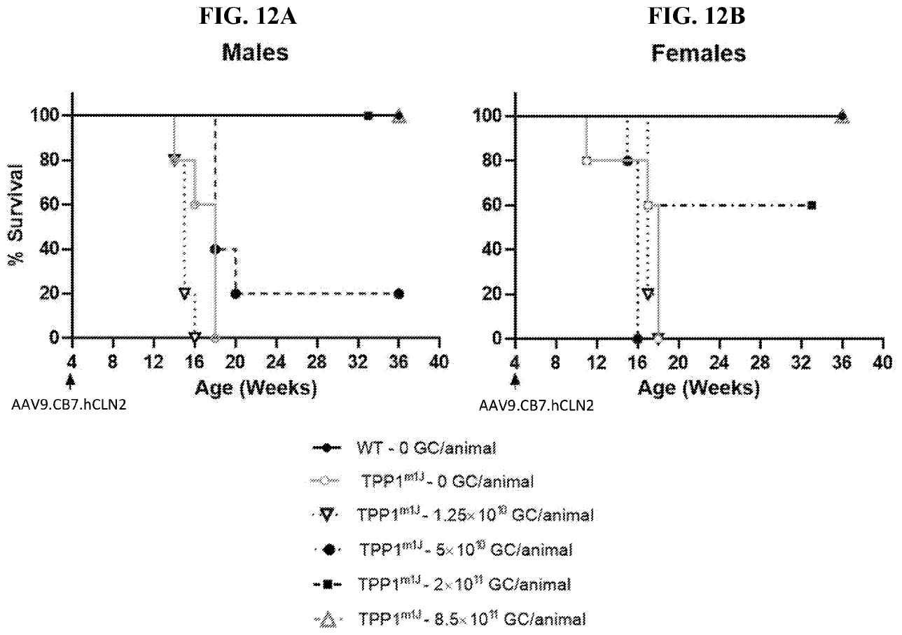

AAV9.CB7.hCLN2 was well tolerated at the dose of 3×10¹¹ GC/animal and increased the lifespan of TPP1^{mlJ} KO mice. The median survival of vehicle-treated TPP1^{mlJ} KO mice was 17.6 weeks for males and 17.4 weeks for females. On average, death occurred 1.5 weeks after the onset of clinically visible signs. At the terminal sacrifice endpoint (16 weeks post-injection, age 23 weeks), 57% of treated males (4 out of 7) and 67% of treated females (4 out of 6) were alive, whereas all untreated TPP1^{mlJ} KO mice were dead by the age of 19 weeks (FIG. 11). AAV9.CB7.hCLN2-treated TPP1^{mlJ} KO mice maintained body weight close to the WT mice throughout the study, whereas vehicle-treated mice progressively lost weight from the age of 13 weeks. The breathing rate was comparable in all groups and no effect of genotype or of treatment could be detected. Normal healthy mice have increased activity at night (dark phase) and low activity during the light phase of the day showing a clear biphasic circadian motion profile. Both male and female, vehicle-treated, TPP1^{mlJ} KO mice started to lose the biphasic profile and showed decreased activity during the dark phase of the day around 16 weeks of age. AAV9.CB7.hCLN2-treated mice presented circadian motion profiles that were identical to WT until the end of the study. Vehicle-treated TPP1^{mlJ} KO mice had decreased night-time motion speed compared with WT mice, with a statistically significant decrease observed at 7.7 weeks post-injection (age 14.7 weeks). On the contrary, AAV9.CB7.hCLN2-treated TPP1^{mlJ} KO mice maintained levels of night-time activity that were statistically significantly higher than vehicle-treated TPP1^{mlJ} KO mice and not statistically significantly different than WT mice. Microscopic examination of the brain at the end of the study showed a decrease in astrocytosis in the cortex and hippocampus of AAV9.CB7.hCLN2-treated TPP1^{mlJ} KO mice at the end of the study.

In this study, the minimum effective dose was considered to be 3×10¹¹ GC/animal, which equates to 7.5×10¹¹ GC/g brain (assuming brain weight of 0.4 g), based on an improved survival and normalization of the disease-related neurobehavioral impairments, namely circadian activity and night-time motion, which correlated with a decrease in neuroinflammation (astrocytosis).

### d) Effects of TPP1 Replacement Using AAV9 Delivery (ICV) in C57BL/6 TPP1mlJ KO Mice

The aim of this study was to establish the minimum effective dose of AAV9.CB7.hCLN2 that increases the lifespan and reduces lysosomal storage material as well as pathology in the brain of mice that lack the TPP1 enzyme. Groups of TPP1^{mlJ} KO mice received a single ICV dose of vehicle or AAV9.CB7.hCLN2 and monitored for survival. An additional group of wild type C57Bl/6 mice were included as controls. This study was divided into two parts, the first part was to evaluate the pharmacodynamics (TPP1 activity) and neuropathology after 9 weeks and the second part was to evaluate the lifespan of these mice. Groups of TPP1^{mlJ} KO mice (n=30/sex/group) were administered a single ICV injection (5 μL) of AAV9.CB7.hCLN2 at doses of 0 (vehicle), 1.25×10¹⁰, 5×10¹⁰, 2×10¹¹, 8.5×10¹¹ GC/animal. Animals (5/sex/group) were euthanized 9 weeks after dosing (13 weeks of age). Animals assigned to the second part of the study to evaluate the effect of AAV9.CB7.hCLN2 on lifespan is ongoing and described below. The following parameters and endpoints were evaluated in this study: mortality, clinical observations, body weight, neurobehavioral observations (predose and Week 8), TPP1 activity, anti-TPP1 antibodies, gross necropsy findings, organ weights and neuropathology (Week 9). Neuropathological examination evaluated neuronal loss in the brain, cervical and lumbar spinal cord as well as intralysosomal accumulation of autofluorescent storage material, astrocytosis (GFAP) and microglial activation (CD68).

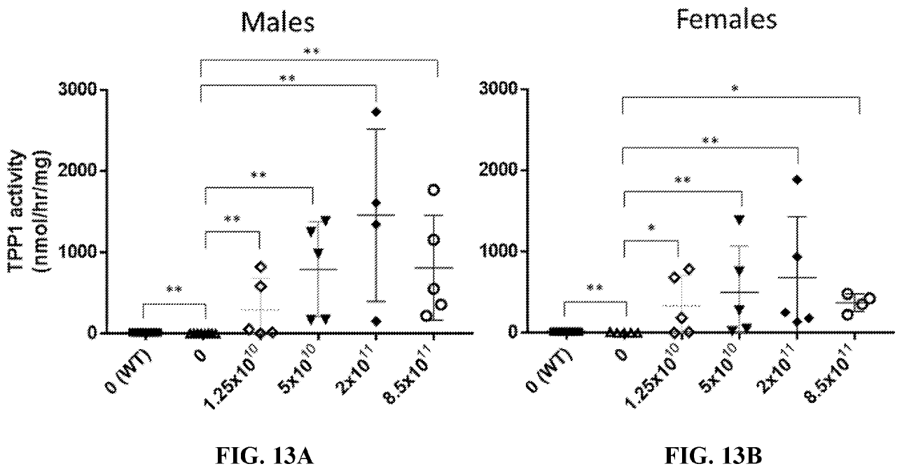

The vehicle and AAV9.CB7.hCLN2 were administered via ICV injection once on Day 1. Clinical signs seen in untreated TPP1^{mlJ} KO mice were generally seen from Week 8 onwards and included tremors and hind limbs splayed typically associated with this disease model. Mortality in this group was between 11 and 18 weeks of age (median of 18 weeks), similar to previous studies with these mice. As shown in FIGS. 12A-12B, at doses ≤5×10¹⁰ GC/animal, there was no improvement in survival. At 1.25×10¹⁰ GC/animal, median survival was 15 and 17 weeks, in males and females, respectively. At 5×10¹⁰ GC/animal, median survival was 18 and 16 weeks in males and females, respectively. At doses ≥2×10¹¹ GC/animal, a clear improvement in survival has been observed with animals currently at 36 weeks of age.

At 40 weeks old, there are 5/5 males and 3/5 females surviving at 2×10¹¹ GC/animal and at 8.5×10¹¹ GC/animal there is 100% survival in males and females. Clinical signs seen in AAV9.CB7.hCLN2-treated animals were comparable to untreated TPP1^{mlJ} KO mice, with additional observations seen including ataxia, teeth chattering, high and low carriage. At 12 weeks of age (Week 8), neurobehavioral observations recorded mild clonic behaviors in the open field and abnormal gait observations in AAV9.CB7.hCLN2-treated animals that were not recorded for untreated TPP1^{mlJ} KO mice. At 20 weeks of age, open field observations included clonic movements in the majority of animals in the two surviving AAV9.CB7.hCLN2-treated groups and convulsions were seen in two animals when they were removed from the home cage. Clonic movement was also noted for 2/5 untreated female WT animals in Week 16. At 30 weeks of age, body weights at ≥2×10¹¹ GC/animal were comparable to WT animals for males and slightly lower for females (−15%). In the 9-week cohort, there were no macroscopic findings or effects on organ weights in either untreated or AAV9.CB7.hCLN2-treated animals.

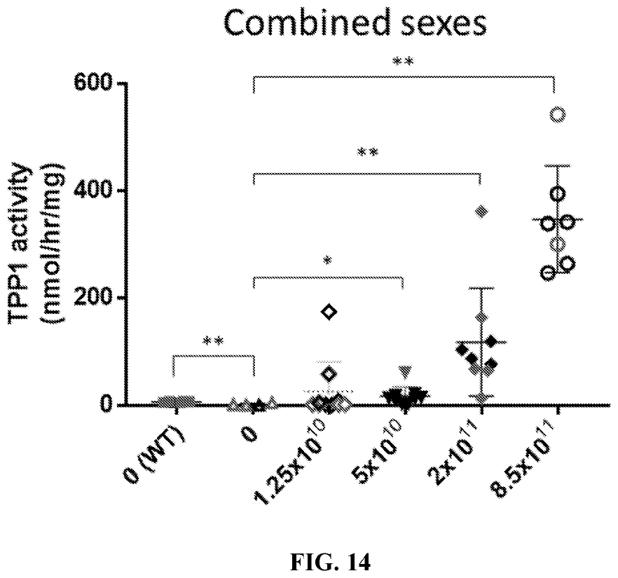

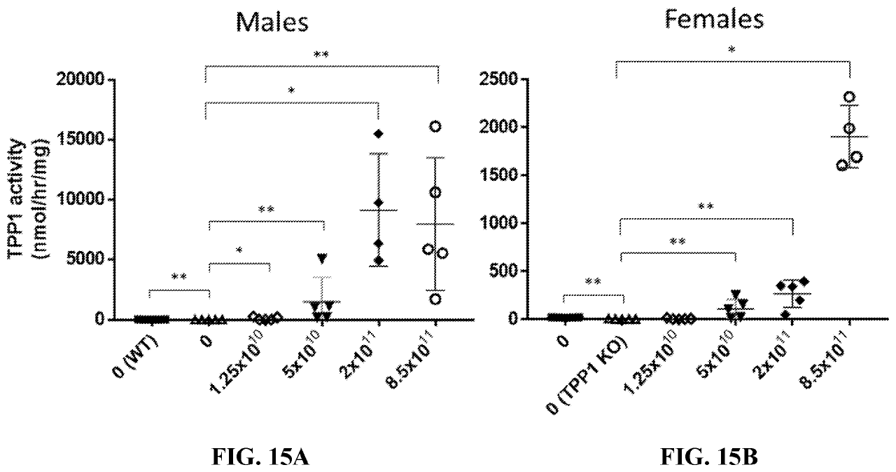

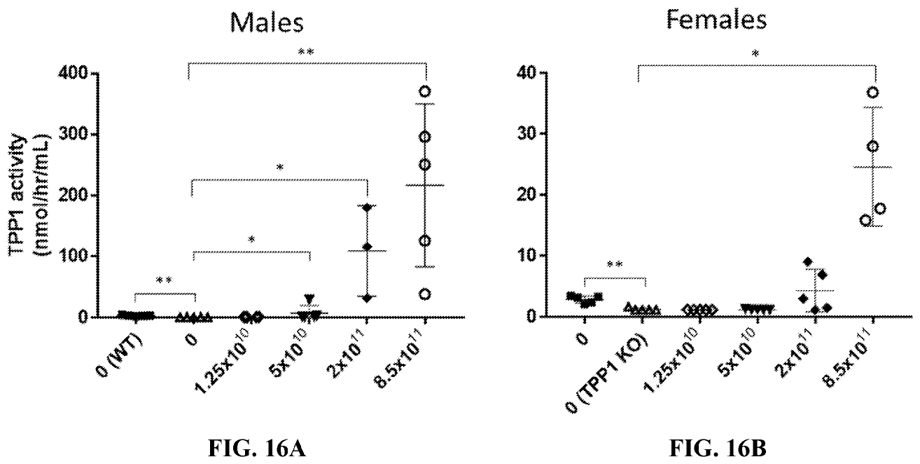

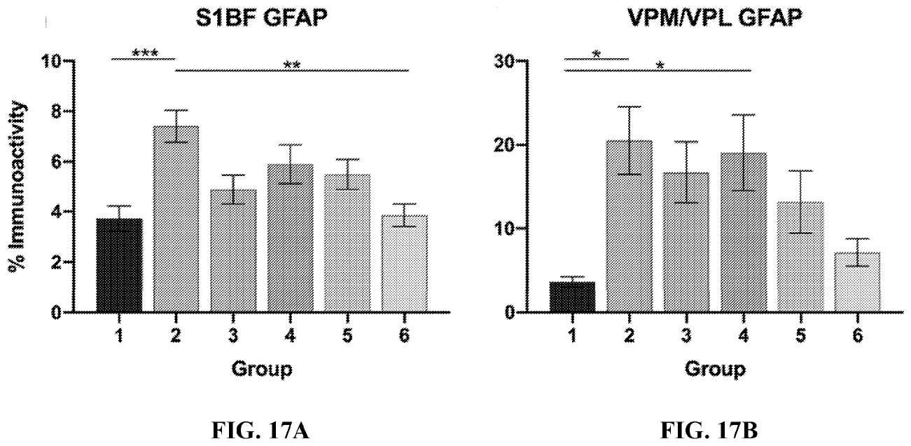

At Week 9, dose-related increases in transgene product (TPP1 activity) were observed in the brain (FIGS. 13A-13B), spinal cord (FIG. 14), liver (FIGS. 15A-15B) and serum (FIGS. 16A-16B). In general, TPP1 activity was greater in males than females, except for spinal cord. The majority of AAV9.CB7.hCLN2-treated animals were positive for anti-TPP1 antibodies. The highest ATPA response was observed at the low dose (1.25×10¹⁰ GC/animal) when compared to the high dose (8.5×10¹¹GC/animal). The difference between the low and high dose is of unknown significance as it may be attributable to the assay at high transgene product concentrations or reflect an induction of immune tolerance.

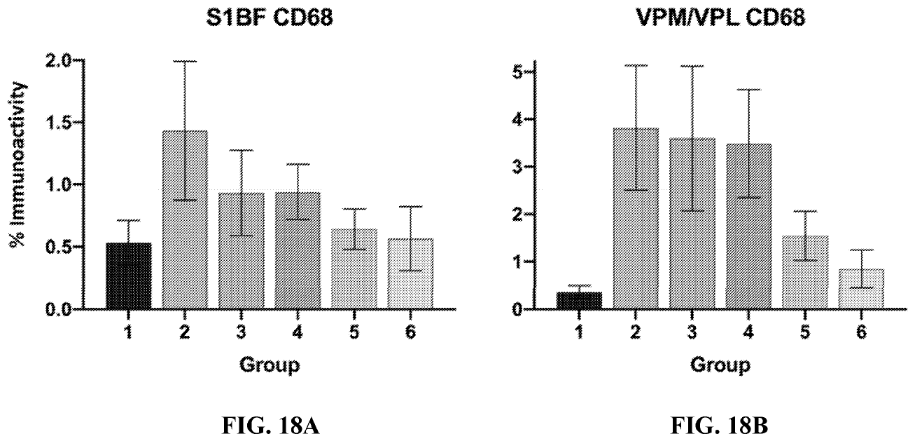

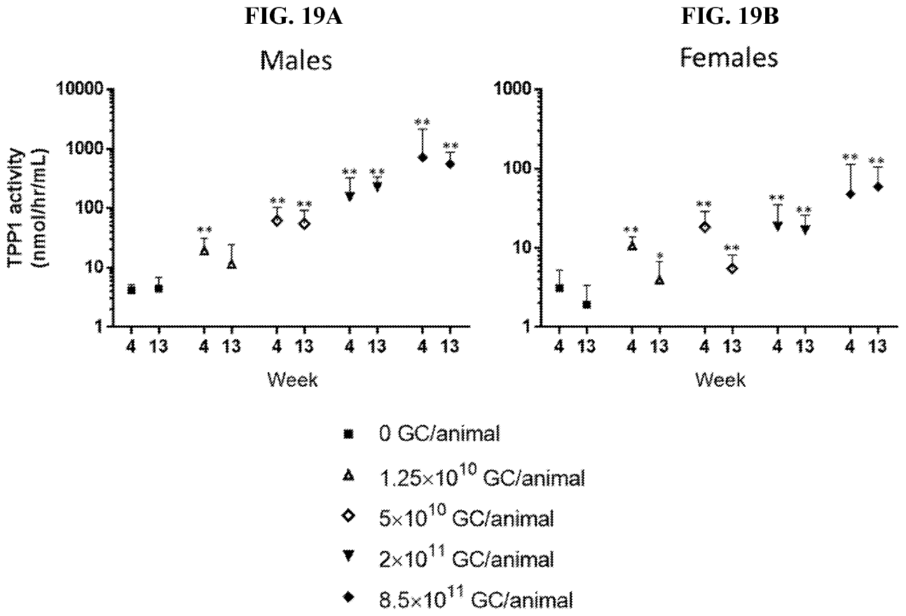

Neuropathological analysis showed a decrease in astrocytosis (FIGS. 17A-17B) and microglial activation (FIGS. 18A-18B) in the cortex and thalamus of mice at ≥2×10¹¹ GC/animal brain that correlated with the ongoing survival. Therefore, the MED in this study was considered to be 2×10¹¹ GC/animal, which equates to a dose of 5×10¹¹ GC/g brain (assuming brain weight of 0.4 g).

This study demonstrated that AAV9.CB7.hCLN2 improved survival in a mouse model of CLN2 disease through transduction of the brain, spinal cord and liver and subsequent reduction in the neuropathological hallmarks of CLN2 disease, astrocytosis and microglial activation. In animals given a dose of 5×10¹⁰ GC/animal, there was similar levels of TPP1 activity to the highest dose, but no improvement in survival in groups with comparable levels of brain TPP1 activity lacked an effect on survival (5×10¹⁰ GC/animal) and improvement in neuropathological endpoints, which could suggest that transduction of the spinal cord and liver were contributing to the overall survival of these animals.

In this study the minimum effective dose was considered to be 2×10¹¹ GC/animal, which equates to 5×10¹¹ GC/g brain (assuming brain weight of 0.4 g), based on improved survival and decreases in astrocytosis and microglial activation.

### 2. Safety Pharmacology

Central Nervous System: Neurobehavioral endpoints were included in the 3-month toxicity study in mice and 4 week pharmacodynamic study in cynomolgus monkeys.

3 month toxicity study in mice: Functional Observational Battery (FOB) evaluations were conducted in Week 13 and included an evaluation of activity, posture, rearing, behavior, response to stimulus (approach, click, tail pinch, and touch), pupil response, grip response and pain perception (latency of response to a nociceptive [thermal]) stimulus). There were no effects on these parameters, with the only finding of note being a decrease in the numbers of rears within the open field in males at ≥2.00×10¹¹ GC/animal and females at 8.50×10¹¹ GC/animal. In the absence of other findings, this was not considered to be AAV9.CB7.hCLN2-related.
4 week pharmacodynamic study in cynomolgus monkeys: In the 4 week study in cynomolgus monkeys, in addition to clinical observations, a comprehensive neurological examination in Week 4 that included general sensory and motor function, cerebral reflexes (pupillary, orbicularis and corneal reflexes) and spinal reflexes (sensory, knee jerk, cutaneous, proprioceptive and tail reflexes) was conducted. There were no AAV9.CB7.hCLN2-related effects on these endpoints and all animals had no behavioral abnormalities or clinical signs during the study.

Respiratory System: Whilst no specific respiratory study was conducted, there were no effects on the respiratory endpoints assessed in the FOB in the mouse toxicity study or microscopic changes in the lungs after 4 or 13 weeks.

Cardiovascular system: No specific cardiovascular studies were conducted and there were no microscopic changes in the mouse toxicity study after 4 or 13 weeks of treatment with AAV9.CB7.hCLN2 indicative of an effect on the cardiovascular system.

### a) Summary

A series of in vivo pharmacology studies were conducted using TPP1^{mlJ} KO mice, a disease model for LINCL Batten disease (CLN2). TPP1^{mlJ} KO mice have a single nucleotide mutation in the splice donor site downstream of exon 8 of the CLN2 gene, which encodes for the soluble lysosomal enzyme TPP1. A natural history study with these mice demonstrated characteristic features of CLN2 disease in humans, including early age at onset of clinical symptoms, rapid progression of the abnormal phenotype, and shortened lifespan, as well as similar pathophysiological, biochemical, and functional changes that were similar to other mouse models of CLN2 disease (Sleat D E, et al. J Neurosci, 2004; 24(41):9117-26; Geraets et al, 2017. PLoS One; 12(5):e0176526). When AAV9.CB7.hCLN2 was administered ICV TPP1^{mlJ} KO to mice, there was an improvement in survival from a median of 18 weeks (untreated) to 40 weeks (study ongoing). An assessment of behavioral endpoints showed an improvement in motor co-ordination and learning as well as restoration of a clear biphasic circadian motion profile in AAV9.CB7.hCLN2-treated TPP1^{mlJ} KO mice. With respect to standard clinical observations, there were no clear differences in onset and clinical signs (tremors and abnormal gait) between untreated and AAV9.CB7.hCLN2-treated animals, yet there has been no progression of these clinical observations and animals continue to gain weight as they progress on the study. In all pharmacology studies, AAV9.CB7.hCLN2 increased TPP1 activity in the brain and spinal cord. The variability observed at high doses in TPP1 activity was of unknown relevance as there were clear dose responses in survival and reduction in neuropathological endpoints. In all studies with AAV9.CB7.hCLN2, there were clear reductions in lysosomal storage material, a normal neuronal cell morphology, and decrease in astrocytosis and microglial activation. The effects of AAV9.CB7.hCLN2, in terms of attenuating the CLN2 neuropathological phenotype via expression of TPP1 are comparable to those described following administration of rhTPP1 to CLN2^{-/-} mice and TPP1-null Dachshunds (Chang et al, 2008; Vuillemenot et al, 2015). In addition to an increase in brain activity, there was an increase in liver TPP1 activity as seen with AAVrh10.hCLN2 (Sondhi et al 2007). As the blood brain barrier is likely to prevent TPP1 leaving the CNS, the activity detected systemically is likely associated to leakage of AAV9.CB7.hCLN2 into the peripheral circulation immediately following dosing. This is potentially beneficial in the treatment of CLN2 disease as systemic TPP1 may attenuate any functional impairment associated with accumulation of lysosomal storage material in tissues and organs outside the CNS (Katz, et al, Gene therapy 2017 Feb. 24(4): 215-223). The gender-related differences observed in the rodent pharmacology studies in transduction efficiency of the liver (TPP1 activity), is considered to be an androgen-dependent effect on the uptake of AAV, independent of serotype, promoter or transgene (Lonning et al, 2002. Molecular Therapy Vol. 5, No. 5; Davidoff et al, 2003. Blood. 15; 102(2):480-8). In the 4-week pharmacodynamic study in cynomolgus monkeys with AAV9.CB7.HCLN2, there were no clear difference between males and females in liver transduction.

In summary, a single dose of AAV9.CB7.hCLN2 in a biologically relevant animal model of disease, was shown to improve survival through increasing TPP1 activity in the brain, spinal cord and liver, attenuate the neuronal loss in the brain, cervical and lumbar spinal cord as well as intralysosomal accumulation of autofluorescent storage material, astrocytosis and microglial activation. In these studies, the minimum effective dose was considered to be a dose of ≥2×10¹¹GC/animal, which equates to 5×10¹¹ GC/g brain.

### B. Pharmacokinetics and Product Metabolism in Animals

An assessment of the pharmacodynamics (transgene product) and immunogenicity was evaluated in the 3-month toxicity study in mice and the 4-week pharmacodynamic study in cynomolgus monkeys.

1. AAV9.CB7.hCLN2: 3-Month Toxicity Study in C57Bl/6 Mice

Groups of C57Bl/6 mice (n=30/sex/group) were administered a single ICV injection (5 μL) of AAV9.CB7.hCLN2 at doses of 0 (vehicle), 1.25×10¹⁰, 5×10¹⁰, 2×10¹¹, 8.5×10¹¹ GC/animal. Compatibility testing using the exact dosing apparatus showed some vector loss at the lower doses, therefore actual doses administered were 0.9×10⁹ (70% recovery), 3.9×10¹⁰ (77% recovery), 1.8×10¹¹ GC/animal (90% recovery) and 8.5×10¹¹ GC/animal (100% recovery) for 1.25×10¹⁰, 5×10¹⁰, 2×10¹¹, 8.5×10¹¹ GC/animal, respectively.

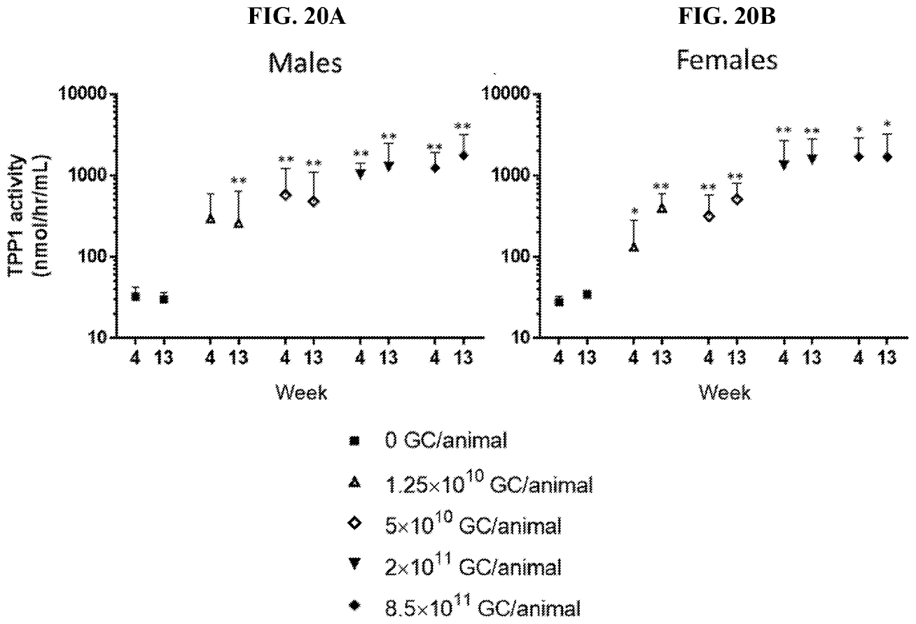

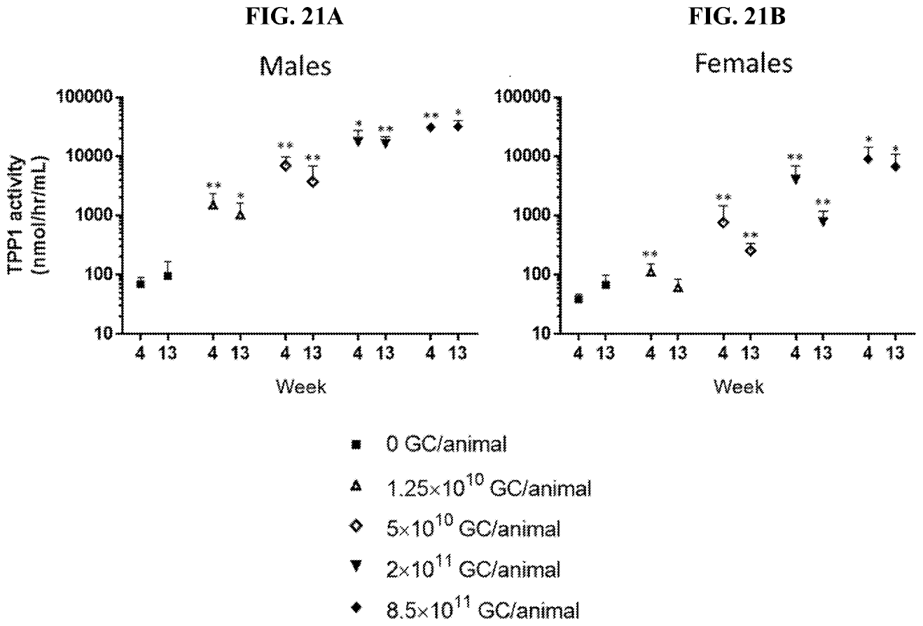

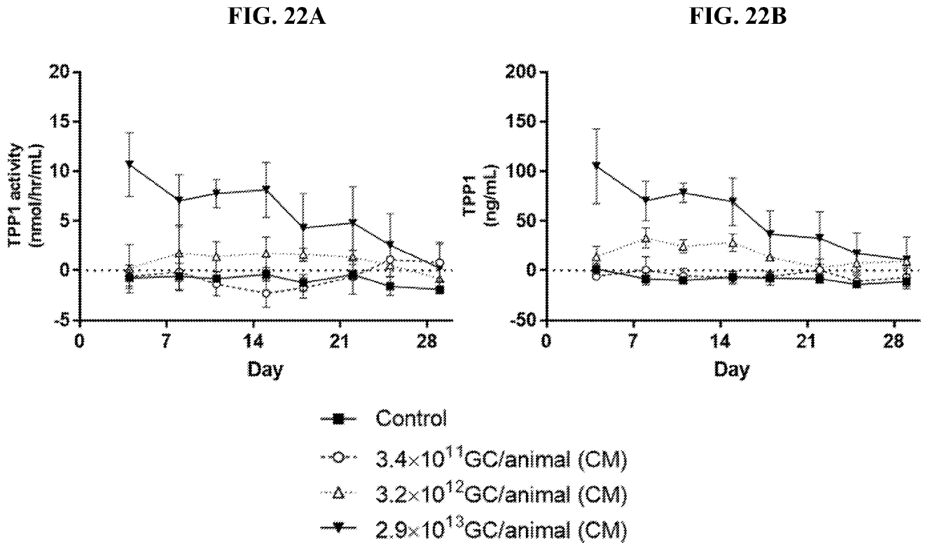

Results are shown in FIGS. 19A-19B, 20A-20B and 21A-21B. Administration of AAV9.CB7.hCLN2 to mice led to dose-dependent increases in brain TPP1 activity in males and females with no sex-related differences in brain TPP1 activity (FIGS. 20A-20B). There were no differences in brain TPP1 activity between Weeks 4 and 13 in both sexes. Liver TPP1 activity was increased in males and females in both Week 4 and 13, with no differences between Week 4 and 13 in males only (FIGS. 21A-21B). However, in females, liver TPP1 activity was consistently lower in Week 13 than Week 4. At the highest dose, 8.5×10¹¹ GC/animal, liver TPP1 activity was 18-(males) and 4 (females)-fold higher than brain TPP1 activity in Week 13. A dose-dependent increase in serum TPP1 activity was observed in males and females (FIGS. 19A-19B). In Week 13, serum TPP1 activity was generally higher in males than females across all dose groups with an approximately 11-fold increase between males and females at 8.5×10¹¹ GC/animal. Serum TPP1 activity in Week 13 was increased in males and decreased in females when compared to Week 4.

The majority of AAV9.CB7.hCLN2-treated animals were positive for anti-TPP1 antibodies in Week 4 and 13. A higher ATPA response was observed at the low dose (1.25×10¹⁰ GC/animal) when compared to the high dose (8.5×10¹¹ GC/animal).

### 2. AAV9.CB7.HCLN2: A Single Dose Pharmacodynamic Study Via Intrathecal Administration in Cynomolgus Monkeys

Groups of cynomolgus monkeys (1 male and 2 females/group) were administered AAV9.CB7.hCLN2 via intrathecal injection via cisterna magna puncture (CM) at doses of 0, 3.4×10¹¹, 3.2×10¹² or 2.9×10¹³ genome copies (GC)/animal (1 mL/animal). An additional group of animals (1 male and 2 females) were administered 3.2×10¹² GC/animal via intrathecal-lumbar puncture (IT-L; 1 mL/animal). Samples of serum and CSF were collected during the study. TPP1 activity and TPP1 concentration was evaluated for the following: serum and CSF (pre-dose [Day −1 or 1], Day 4, 8, 11, 15, 18, 22, 25 and 29), liver, spinal cord (cervical, thoracic and lumbar regions) and brain. For the brain, superficial or deep samples were analyzed from the frontal cortex, occipital cortex, cerebellum, striatum, medulla oblongata, midbrain and thalamus.

Six animals out of 15 were deemed positive for anti-transgene product (TPP1) antibody (ATPA) responses at the pre-dose sample time-point. On Day 29, the incidence of ATPA positive animals was 1/3 animals at 0 GC/animal, 2/3 animals at 3.4×10¹¹ GC/animal (CM), 3/3 animals at 3.2×10¹² GC/animal (CM), 3/3 animals at 2.9×10¹³ GC/animal (CM) and 2/3 animals at 3.2×10¹² GC/animal (IT-L).

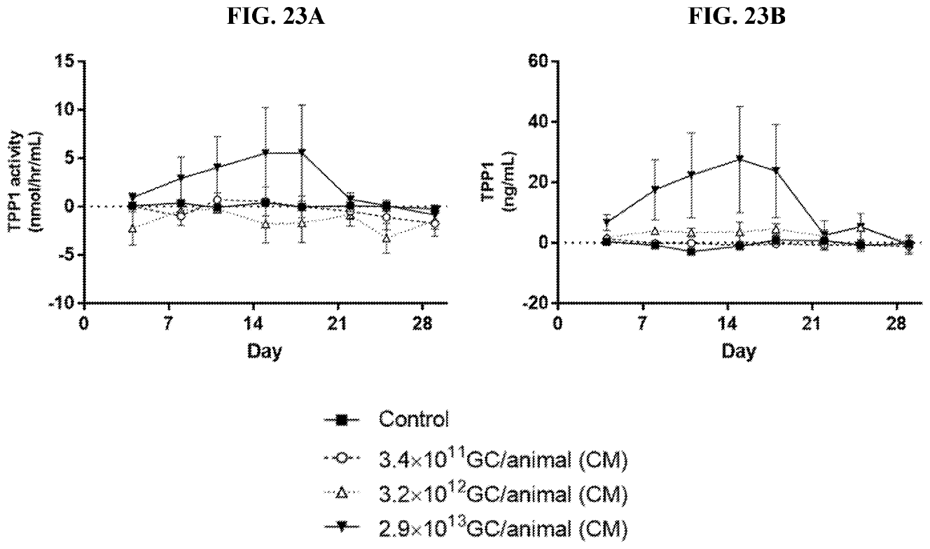

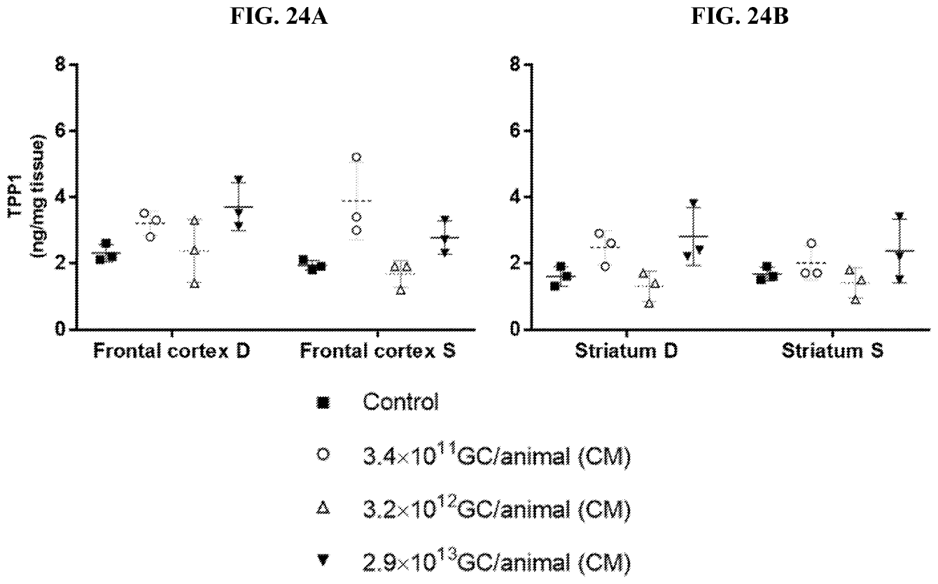

At 2.9×10¹³ GC/animal (CM), there were increases observed in TPP1 activity and concentration in both serum and CSF, with peak levels generally seen on Day 15 (FIGS. 22A-22B and 23A-23B). Thereafter, there was a trend for a decline in TPP1 activity and concentration towards the end of the study. The decline in TPP1 activity and concentration in the serum and CSF was associated with the immunogenicity observed in these animals at the end of the study and typically seen in non-human primates administered recombinant human proteins. At 3.2×10¹² GC/animal (CM), there were minimal increases in mean serum TPP1 activity and concentration, when compared to the baseline value which declined from Day 18 onwards, again likely to be associated with immunogenicity to the human transgene product. There were no clear increases in CSF TPP1 activity or concentration observed in this group, although one animal did show a small increase between Days 8 and 25. At 3.4×10¹¹ GC/animal (CM), there were no increases in TPP1 activity or concentration observed in the serum or CSF.

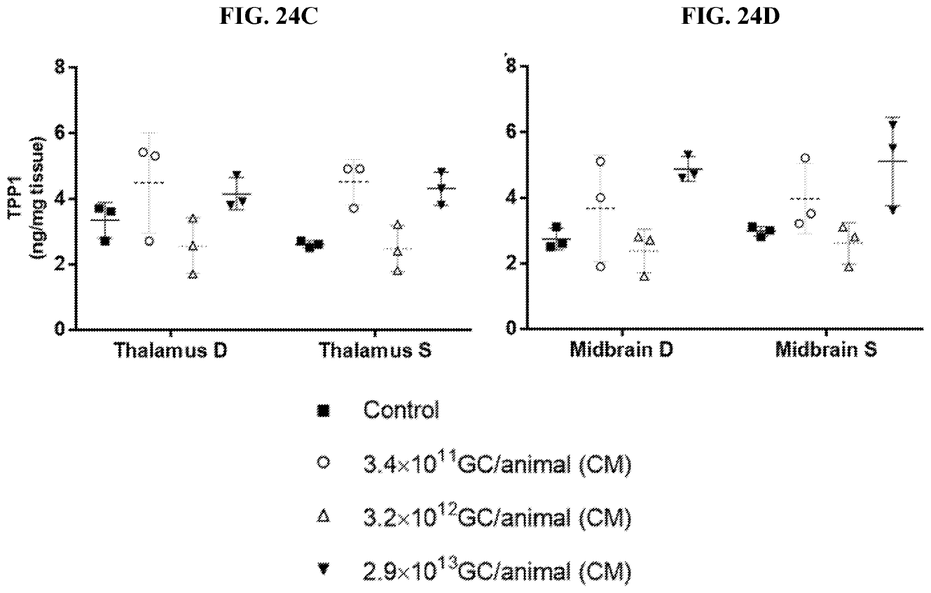

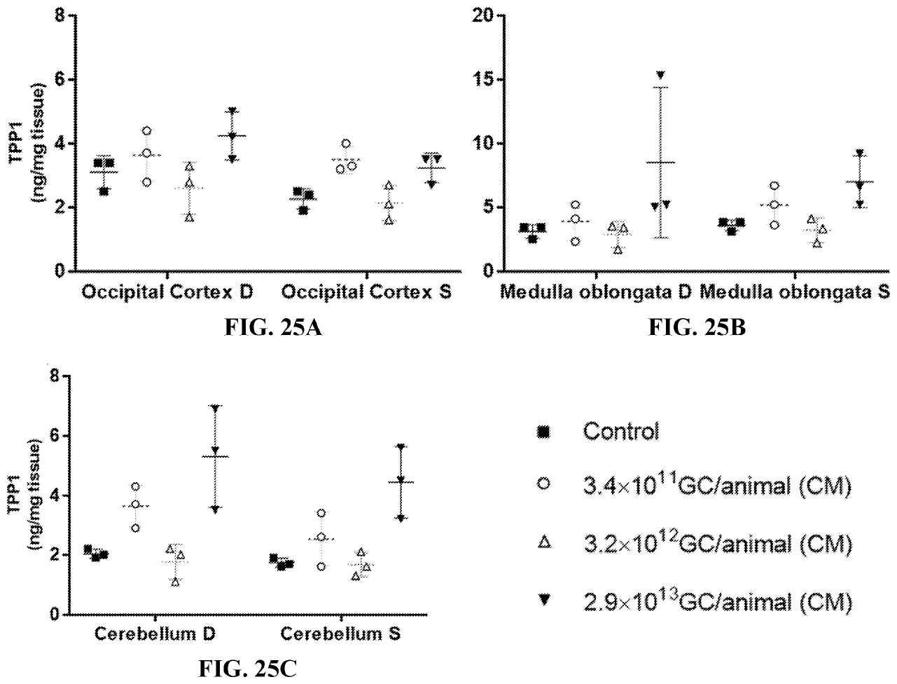

At 2.9×10¹³ GC/animal (CM), mean TPP1 activity was increased when compared to the control group for the midbrain (deep [1/3 animals] and superficial), medulla oblongata (deep and superficial) and cerebellum (deep and superficial). At this dose, mean TPP1 concentrations were increased when compared to the control group for the frontal cortex (deep and superficial), striatum (deep and superficial), thalamus (superficial), midbrain (deep and superficial), occipital cortex (deep and superficial), medulla oblongata (deep and superficial) and cerebellum (deep and superficial; Table 3; FIGS. 24A-24D and 25A-25C). At 3.2×10¹² GC/animal, there were no clear differences in TPP1 activity or TPP1 concentrations when compared to the control group. One animal did show trends for small elevations in most of the deep brain regions when compared to the control mean value. For animals at the lowest dose (3.2×10¹² GC/animal), there were increases observed in all brain regions when compared to the control group, however in the absence of a dose-response and that values were, in general, lower than the high dose, the relationship to treatment is uncertain.

TABLE 3
TPP1 concentration in brain regions of AAV9.CB7.hCLN2-
treated (CM) cynomolgus monkeys

| | Fold Versus Control | | |
|---|---|---|---|
| Dose (GC/animal) | 3.4 × 10¹¹ | 3.2 × 10¹² | 2.9 × 10¹³ |
| Adjusted dose (GC/g brain)^{A} | 5.26 × 10⁹ | 4.59 × 10¹⁰ | 4.20 × 10¹¹ |
| Brain region | | | |
| Frontal cortex | | | |
| Deep | x1.39 | x1.04 | x1.61 |
| Superficial | x2.05 | x0.89 | x1.47 |
| Striatum | | | |
| Deep | x1.56 | x0.81 | x1.75 |
| Superficial | x1.18 | x0.82 | x1.41 |
| Thalamus | | | |
| Deep | x1.36 | x0.79 | x1.24 |
| Superficial | x1.73 | x0.96 | x1.65 |
| Midbrain | | | |
| Deep | x1.33 | x0.89 | x1.81 |
| Superficial | x1.38 | x0.90 | x1.76 |
| Occipital cortex | | | |
| Deep | x1.16 | x0.84 | x1.35 |
| Superficial | x1.52 | x0.91 | x1.39 |
| Medulla oblongata | | | |
| Deep | x1.26 | x0.90 | x2.74 |
| Superficial | x1.44 | x0.89 | x1.94 |
| Cerebellum | | | |
| Deep | x1.80 | x0.90 | x2.65 |
| Superficial | x1.44 | x0.94 | x2.5 |

^{A}Adjusted for group mean brain weight on Day 29

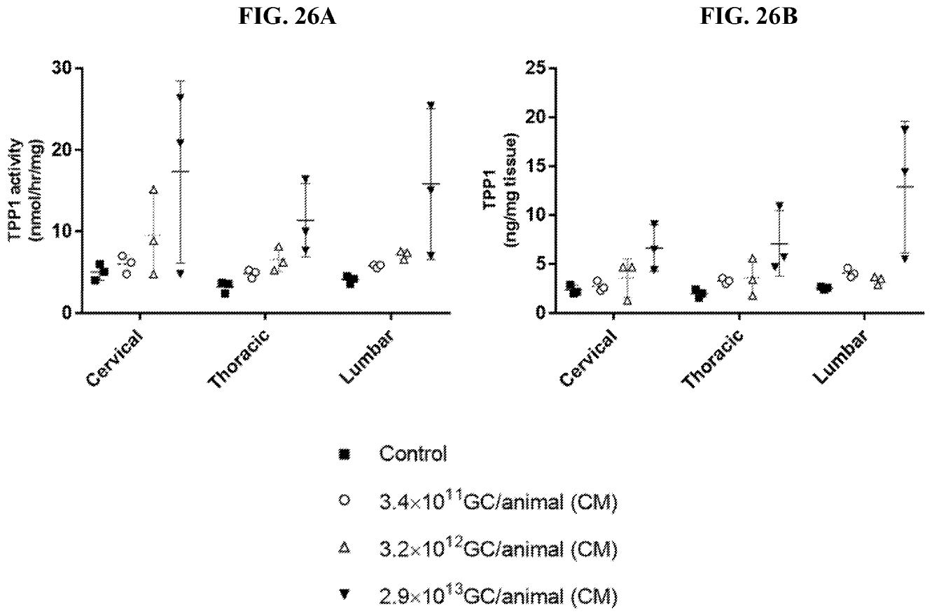

At 2.9×10¹³ GC/animal, TPP1 activity was increased in all regions (cervical, thoracic and lumbar) of the spinal cord when compared to the control group. In 2/3 animals these changes were up to 5.3-fold greater than the control group. The remaining animal in this group still showed increases in TPP1 activity, however this was less than the other two animals, being up to 2.4-fold greater than the control group. The increases in TPP1 concentrations followed the profile of TPP1 activity with increases observed in all regions (cervical, thoracic and lumbar) of the spinal cord. Whilst TPP1 activity did not show differences between the different regions of the spinal cord, TPP1 concentrations were greater in the lumbar region of the spinal cord, when compared to the cervical or thoracic regions. At 3.2×10¹² GC/animal, TPP1 activity was increased in all regions (cervical, thoracic and lumbar) of the spinal cord with an increase of up to 2-fold when compared to the control group (FIGS. 26A-26B). This was also reflected in TPP1 concentrations where there were increases up to 1.8-fold observed. At 3.4×10¹¹ GC/animal, TPP1 activity and TPP1 concentrations were increased in the cervical, thoracic and lumbar regions of the spinal cord, with an increase of up to 1.6-fold when compared to the control group.

At 2.9×10¹³ GC/animal, TPP1 activity and TPP1 concentrations were increased by 7-fold and 4-fold, respectively. At 3.2×10¹² GC/animal, TPP1 activity and TPP1 concentrations were increased by 3.7-fold and 1.5-fold, respectively. At 3.4×10¹¹ GC/animal, TPP1 activity and TPP1 concentrations were increased by 1.8-fold and 1.6-fold, respectively.

At 3.2×10¹² GC/animal (IT-L), there were no clear differences in serum TPP1 activity when compared to the control group over the course of the study. This was not reflected when serum TPP1 concentrations were evaluated as minor increases were observed from Day 4 onwards until the end of the study. For CSF, TPP1 activity was comparable to the control group, however TPP1 concentrations did show a small increase from Day 8 onwards when compared to baseline, which was not observed for the equivalent dose that was administered CM. A trend was observed for minimal elevations in TPP1 activity for the frontal cortex (deep and superficial), striatum (deep and superficial), thalamus (deep and superficial), midbrain (deep and superficial), occipital cortex (deep and superficial), medulla oblongata (deep and superficial) and cerebellum (deep and superficial). This was also seen, to a lesser extent, for TPP1 concentration, and was mainly due to small increases in one or two animals. The relationship to treatment is unclear as the increases were minimal and not observed in all animals. There were no clear differences between animals receiving AAV9.CB7.hCLN2 via CM or IT-L at this dose, with the exception of the spinal cord. In general, both TPP1 activity and concentration were greater in the spinal cord of IT-L treated animals, in particular the cervical and lumbar regions when compared to the CM group at the same dose. The increase in the cervical region of spinal cord, may be associated to animals in this group being placed in the Trendelenburg position immediately after dosing.

### 3. Summary

An assessment of TPP1 (transgene product) in the 3-month mouse toxicity study showed a dose-related increase in TPP1 activity in the brain, liver and serum. As with the pharmacology studies, liver TPP1 activity was greater in males than females which was not apparent in the cynomolgus monkey. There were no clear differences between 1 and 3 months, indicating peak transgene expression by 4 weeks. In this study, and as seen in the pharmacology studies, there was a high incidence of anti-TPP1 antibodies, with the lowest values observed at the high dose which may be attributable to assay interference or reflect an induction of immune tolerance.

In the 4 week pharmacodynamic study, administration of AAV9.CB7.hCLN2 led to increases in TPP1 activity and concentration in the serum and CSF at a dose 2.9×10¹³ GC/animal, which equates to a group mean dose of 4.20×10¹¹ GC/g brain (based on brain weight at the end of the study). The decrease in TPP1 seen in the CSF and serum by Week 2 was associated with immunogenicity to the human transgene product, similar to an effect generally seen in nonclinical studies with biotherapeutics. Whilst a decrease in TPP1 levels were seen in the CSF and serum, it is unclear if the presence of anti-Tpp1 antibodies also could have influenced the TPP1 levels in the brain and underestimated the concentration due to increased clearance or other mechanisms (neutralizing antibodies).

At the highest dose in this study, there was an increase in TPP1 concentration in the brain regions on Day 29 similar to that published for recombinant human TPP1 (rhTPP1) in cynomolgus monkeys (Vuillemenot et al, 2014). The main difference between AAV9.CB7.hCLN2 and rhTPP1 given ICV, is that AAV9.CB7.hCLN2 transduced cells constantly produce TPP1 which is almost equivalent to giving rhTPP1 by continuous infusion and achieving a zero-order pharmacokinetic profile. Therefore, upon AAV9.CB7.hCLN2 administration there will not be such a peak to trough for TPP1 in the brain and CSF as seen when rhTPP1 is delivered ICV. The lack of a clear effect at lower doses in this study suggested that minimum dose in cynomolgus monkeys to see increases in TPP1 in the brain might be 4.20×10¹¹ GC/g brain of AAV9.CB7.hCLN2. In this study, there was transduction of the spinal cord, which may account for the increase in TPP1 in the CSF, as the two animals with the greatest TPP1 activity/concentration in the spinal cord were also the animals observed with the highest CSF TPP1 concentrations. As with the pharmacology studies in TPP1^{mlJ} KO mice, there were increases in the transgene product in the liver and serum. This was associated to AAV9.CB7.hCLN2 distribution into the peripheral circulation immediately following dosing and might be potentially beneficial in the treatment of CLN2 disease as described previously. As there were no clear effects in this study at low doses, the data from cynomolgus monkeys suggested that a dose of 2.9×10¹³ GC/animal, which equates to a dose of 4.20×10¹¹ GC/g brain (dose calculated from brain weight at the end of the study) might be required to see increases in TPP1 activity in the brain and spinal cord and that these doses were similar to the minimum effect dose that was achieved in the mouse pharmacology studies.

### C. Toxicology

### 1. Single Dose Studies

a) AAV9.CB7.hCLN2: 3-Month Toxicity Study in C57Bl/6 Mice

The objective of this study was to evaluate the pharmacodynamics, toxicity, and immunogenicity of AAV9.CB7.hCLN2 in C57Bl/6 mice following a single ICV dose. Groups of mice (n=30/sex/group) were administered a single ICV injection (5 μL) of AAV9.CB7.hCLN2 at doses of 0 (vehicle) 1.25×10¹⁰, 5×10¹⁰, 2×10¹¹, 8.5×10¹¹ GC/animal. Animals were euthanized after either 4 (10/sex/group) or 13 (10/sex/group) weeks after dosing. An additional group of satellite animals (n=5/sex/group) was euthanized at each time-point to evaluate transgene product (TPP1 activity) in the brain and liver. Compatibility testing using the exact dosing apparatus showed some vector loss at the lower doses, therefore actual doses administered were 0.9×10⁹ (70% recovery), 3.9×10¹⁰ (77% recovery), 1.8×10¹¹GC/animal (90% recovery) and 8.5×10¹¹ GC/animal (100% recovery) for 1.25×10¹⁰, 5×10¹⁰, 2×10¹¹, 8.5×10¹¹ GC/animal, respectively.

The following parameters and end points were evaluated in this study: mortality, clinical observations, body weight, food consumption, ophthalmoscopic examinations, functional observational battery (FOB) evaluations, clinical pathology parameters (hematology and clinical chemistry), TPP1 enzyme activity (serum, brain and liver) and anti-TPP1 antibodies (serum), gross necropsy findings, organ weights, macroscopic and microscopic examination.

There were no clear treatment-related observations in mice given a single ICV dose of AAV9.CB7.hCLN2 at doses up to 8.5×10¹¹ GC/animal over 90 days. Three of the 60 animals administered AAV9.CB7.hCLN2 at 8.5×10¹¹ GC/animal were found dead (2/3) or were euthanized (1/3) in extremis within a week following dosing and the cause of death was not determined. Clinical signs in the male animal euthanized in extremis on Day 4 included thin appearance, decreased activity, ataxia, cold to touch, hunched posture, piloerection and severe dehydration alongside microscopic changes indicative of stress suggesting the animal did not recover fully from the ICV injection. All other microscopic changes in this animal were seen in animals euthanized in Week 4 or 13. As these mortalities occurred in the high dose only, the relationship to treatment is uncertain.

Following the FOB evaluation in Week 13, a marked decrease in the numbers of rears within the open field was observed in Week 13 in males at ≥2.00×10¹¹ GC/animal and females at 8.50×10¹¹ GC/animal. There were minor effects on hematology (decreases in MCV, hematocrit and increases in MCHC and RDW) and clinical chemistry (decreases in urea nitrogen, creatinine and triglyceride concentrations) parameters observed at Week 4 which partially resolved by Week 13. At 8.50×10¹¹ GC/animal, a decrease in albumin (X0.9) was seen in males and females in Week 4 and females only in Week 13.

Microscopic changes were observed in brain, spinal cord and sciatic nerve following administration of AAV9.CB7.hCLN2. At 8.50×10¹¹ GC/animal only, unilateral brain necrosis (at putative injection site) with associated inflammation (subacute/chronic) was noted in 1/10 male and 3/10 females in Week 4 (mild to moderate) and 1/10 male and 2/10 females in Week 13 (minimal to moderate). Perivascular mononuclear cell infiltration (minimal to moderate) was noted in the brain of mice at 2.00×10¹¹ (2/10 males and 2/10 females) and 8.50×10¹¹ (3/10 males and 7/10 females) GC/animal in Week 4. In Week 13, this was observed in fewer mice (1/10 males and 5/10 females) at 8.50×10¹¹ GC/animal only. These infiltrates were considered an immune response to necrosis, inflammation, and/or the test article.

In the spinal cord, axonal degeneration (minimal to moderate) of the peripheral nerve roots was seen in mice at ≥2.00×10¹¹ GC/animal in Week 4 and 13. In the sciatic nerves, axonal degeneration (minimal to moderate) was seen in mice at ≥5.00×10¹⁰ GC/animal in Week 4 and 13. Axonal degeneration in the sciatic nerve (minimal) was also observed in the male killed on Day 4. These findings were present in multiple nerve sections, suggesting that the findings were bilateral in distribution. Axonal degeneration was characterized by swollen eosinophilic axons, axonal fragmentation, formation of digestion chambers with phagocytic macrophages and associated reactive gliosis.

AAV9.CB7.hCLN2-related non-adverse findings occurred in the spleen the mice at ≥1.25×10¹⁰ GC/animal in Week 4 and 13 that consisted of minimal to mild lymphoid hyperplasia (germinal centers and marginal zones), and increased incidence and/or severity of cellularity in the red pulp, hematopoietic cells, and/or marginal zone hyperplasia (minimal to mild). These findings correlated with the increased spleen weights. In Week 13, the changes in the spleen were not detected in male animals only at the high dose.

Based on the findings seen in the sciatic nerve and nerve roots of the spinal cord, the No Observed Adverse Effect Level (NOAEL) was considered to be 1.25×10¹⁰ GC/animal.

b) AAV9.CB7.hCLN2: A Single Dose Pharmacodynamic Study Via Intrathecal Administration in Cynomolgus Monkeys

The objective of this study was to evaluate the pharmacodynamics and toxicity of AAV9.CB7.hCLN2 (AAV9.hCLN2) in cynomolgus monkeys after 4 weeks. Groups of cynomolgus monkeys (1 male and 2 females/group) were administered AAV9.CB7.hCLN2 via intrathecal injection via cisterna magna puncture (CM) at doses of 0, 3.4×10¹¹, 3.2×10¹² or 2.9×10¹³ genome copies (GC)/animal (1 mL/animal). An additional group of animals (1 male and 2 females) were administered 3.2×10¹² GC/animal via intrathecal-lumbar puncture (IT-L; 1 mL/animal). At the end of the study, animals were euthanized on Day 29. Endpoints included: clinical observations, body weight, food consumption (qualitative), neurological examinations (general sensory and motor function, cerebral reflexes and spinal reflexes) pharmacodynamics (TPP1 activity and concentration), CSF total cell count, organ weights, macroscopic and microscopic examination (brain [spanning the forebrain to brain stem], spinal cord with attached spinal nerve roots and ganglion [cervical, thoracic, lumbar and lumbosacral regions], sciatic nerve and trigeminal ganglia). For pharmacodynamic evaluation, samples of serum, CSF, liver, spinal cord (cervical, thoracic and lumbar regions) and brain (superficial or deep samples were analyzed from the frontal cortex, occipital cortex, cerebellum, striatum, medulla oblongata, midbrain and thalamus) were evaluated.

There were no treatment-related clinical signs, effects on body weight or physical and neurological examinations observed during the study. At the end of the study, there were no meaningful changes in CSF cell counts.

At 2.9×10¹³ GC/animal (CM), there were microscopic changes observed in the dorsal root ganglia (DRG), predominantly the lumbosacral DRGs of ⅔animals. The adverse microscopic changes in DRG were neuronal degeneration (minimal to mild) and increased cellularity, predominantly infiltrates of lymphocytes/macrophages (moderate to marked) associated with an increased severity of degeneration in spinal nerve roots, dorsal nerve roots (⅓animals only) and dorsal spinal cord tracts in the lumbar (moderate) and lumbosacral (moderate) regions. In a single animal at this dose, microscopic changes (neuron degeneration, gliosis) in the brain were also considered to be potentially adverse, although the changes in the brain were so slight no nervous system dysfunction would be expected. Neuronal degeneration in the brain was considered unlikely in the other animals on this study because there was no evidence of a glial response. While necrotic neurons were cleared quickly, typically there was a lasting glial response that could be detected. In the two affected animals, the DRG findings were associated with a greater degree of transduction (as shown by TPP1 activity and concentration) compared to the animal that was not affected. TPP1 activity in the lumbar region in these animals was more than 2-fold greater than the unaffected animal.

At 3.2×10¹² GC/animal (CM), there were microscopic changes, predominantly in the lumbosacral DRGs of ⅓animals observed. Changes in the DRG were neuronal degeneration (moderate) and increased cellularity, predominantly infiltrates of lymphocytes/macrophages (marked) associated with an increased severity of degeneration in spinal nerve roots of lumbar and lumbosacral region (moderate) as well as dorsal nerve roots in lumbosacral (moderate) region.

At 3.2×10¹² GC/animal (IT-L), changes in DRG were neuronal degeneration in the lumbosacral region (mild) and increased cellularity, predominantly infiltrates of lymphocytes/macrophages in the lumbar and lumbosacral region (moderate). In one animal, increased cellularity (marked) and neuronal degeneration (minimal) was observed in the cervical region of the DRG. In the same animal, an increased severity of degeneration (moderate) in spinal nerve roots of lumbosacral region and degeneration (mild) in the dorsal nerve roots of the cervical region. The changes in the cervical region of this animal, might have been associated with the greater degree of transduction (i.e. TPP1 activity and concentration) of the cervical region, when compared to the IT-CM group. This animal had the highest TPP1 activity in the cervical region, being approximately 3-fold higher than the other two animals in this group. It was also noted in this group, that the two animals with the highest TPP1 activity in the lumbar region of the spinal cord were seen with degeneration in the lumbar/lumbosacral DRG.

Whilst degeneration in spinal nerve roots, dorsal nerve roots and dorsal spinal cord tract seen in treated animals at 2.9×10¹′ or 3.2×10¹² GC/animal might be associated with placement of an IT catheter and was observed in control animals, a portion of the degeneration at this level was attributed to increased cellularity and neuron degeneration in DRG in some ganglia.

The exact cellular constituency of the increased cellularity observed in some DRGs was not determined. The morphologic appearance suggested a combination of an increase of satellite glial cells with infiltrates of lymphocytes and macrophages. The IBA-1 stain (for microglia) of spinal cord sections verified the macrophage component of the infiltrate in some ganglia present on the spinal cord sections of multiple animals. It was suspected that the predominant cell type making up the increased cellularity was infiltrating lymphocytes. There was no clear correlation of animals that were ATPA positive with the occurrence of the findings in the DRG. At the highest dose, all animals were ATPA positive, yet the DRG changes were seen in only 2/3 animals. However, in the one animal that was negative post dose at 3.2×10¹² GC/animal (IT-L) did not show DRG findings.

At 3.4×10¹¹ GC/animal, there were no test article-related changes. Therefore, based on the findings in the DRG, the no observed adverse effect level (NOAEL) was considered to be 3.4×10¹¹ GC/animal.

### 2. Genotoxicity

AAV9.CB7.hCLN2 is a recombinant AAV that does not contain the integration machinery of WT AAV. Once the AAV is internalized into the host cell, it undergoes transport into the nucleus and uncoating to form circularized episomes that persist in the nucleus. AAV9.CB7.hCLN2 is unable to replicate in transduced cells because the source helper plasmid used to produce the product does not contain the necessary cis elements critical for replication. This is contrary to WT AAV, which, in addition to existence of the rep elements, also require active helper virus to reproduce.

Based on the available scientific literature and clinical data, integration of AAVs has not been observed. There are a few articles suggestive of an increased incidence of liver tumors in AAV treated mice; however, vector DNA was low in these tumors and lacked expression of transgene product (Bell P, et al. Mol Ther 2006; 25:34-44). A recent study reported that tumor incidence was very similar between AAV9-treated MPS II mice and non-treated WT mice (38% and 36%, respectively) (Fu H, et al. Mol Ther Methods Clin Dev. 2018; 10:327-340). Therefore, the weight of evidence from recent studies alongside the recently approved AAV9, onasemnogene abeparvovec-xioi, supports that rAAVs have a low risk for insertional mutagenicity.

### 3. Carcinogenicity Studies

In the 3-month mouse toxicity study with AAV9.CB7.hCLN2, no pre-neoplastic or neoplastic lesions were observed.

### 4. Other Toxicity Studies

a) Immunotoxicity

Data from the toxicity studies with AAV9.CB7.hCLN2 in mice and monkeys showed that there were no AAV9.CB7.hCLN2-related effects on leukocytes, or morphological changes in the spleen, lymph nodes or thymus indicative of immunotoxicity (as per ICH S8). The changes in the spleen observed in the mouse toxicity study may be considered to be an adaptive response to the observed immunogenicity to the human transgene product.

### b) Summary

The nonclinical safety of AAV9.CB7.hCLN2, including pharmacodynamics (transgene product) and immunogenicity (anti-TPP1 antibodies) was evaluated in single dose toxicity studies in cynomolgus monkeys and mice for up to 1 and 3 months duration, respectively. In both these studies, there were no AAV9.CB7.hCLN2-related adverse in life findings. Principal findings in both the mouse and cynomolgus monkey were axonal degeneration in the spinal cord, dorsal root ganglia and sciatic nerve.

### Example 3: Free/Thaw Study of Pharmaceutical Compositions Comprising rAAV

### I. Introduction

Freezing and thawing rates can impact the stability of biologics (Cao et al., 2003, Biotechnol. Bioeng. 82(6):684-90). Crystallization of water during slow freezing can result in concentration of excipients which can impact the stability of biologics. Phase separation or pH shifts may also occur with an impact the stability of biologics. Fast freezing can lead to smaller ice crystals and a larger ice-water interface area which could impart interfacial stresses. Fast freezing could also entrap air bubbles in the ice leading to air-water interfacial stress during thawing. Slow thawing can result in re-crystallization of ice which can impact the stability of biologics in solution due to interfacial stress.

Freeze-thaw stress can potentially disrupt AAV capsids resulting in release of small amounts of free DNA. During clinical trials, FDP is shipped between the multiple vendors used for fill finish, storage, clinical packaging and labelling, and is ultimately delivered to clinical sites. Un-planned temperature excursions encountered during shipment or product handling could lead to product warming or even thawing and re-freezing. The relative impacts of the rates of freezing and thawing could be used to assess excursions as well as guide freezing and thawing instructions at CMOs and at the clinic. The impact of freezing and thawing may also depend on the AAV type and its formulation. These factors were assessed in this study.

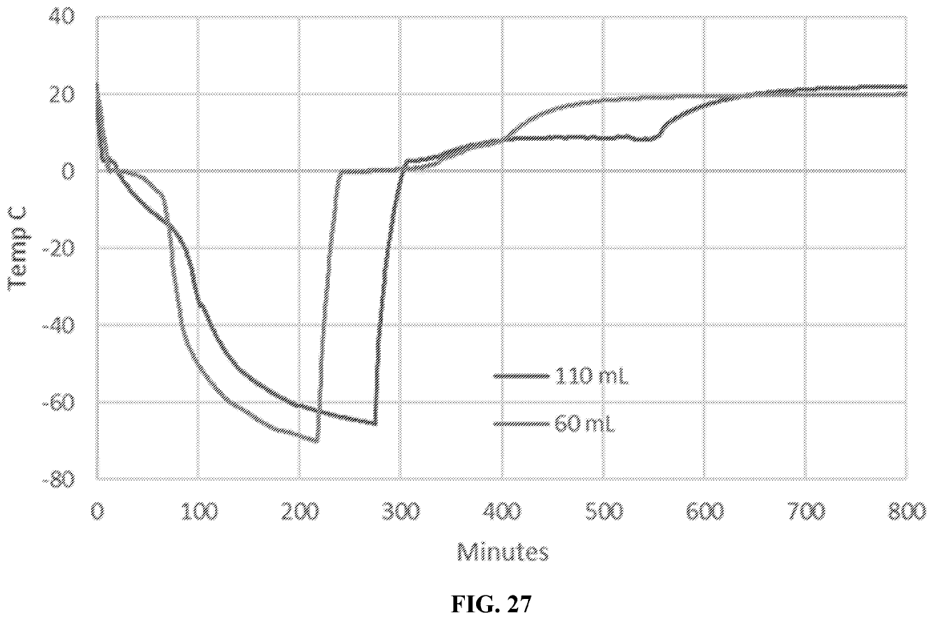

Larger volumes (60-110 mL) in BDS bottles were found to freeze at an overall average rate of about 0.5° C./min and 0.3° C. /min for 60 and 110 mL of water, respectively. Smaller 0.6 mL fills in CZ vials took about 30-40 minutes to freeze from either room temperature or −20° C. (rates of about 2.0° C. /min) while thawing took 30 minutes from room temperature and 10 minutes from −20° C. (rates of about 2.4° C./min and 4.5° C. /min, respectively). In a prior study, it was shown that 250 mL Nalgene HDPE (BDS) bottles filled with 60 and 110 mL of water, took 163 and 266 minutes to freeze below −65° C., respectively. The corresponding freeze rates for these volumes were 0.53° C./min and 0.33° C./min for 60 and 110 mL of water, respectively. During thawing, a rapid rise in temperature was observed until the melting point was achieved—at which point the temperature increased slowly toward room temperature. Thawing took 273 and 337 minutes for 60 and 110 mL bottles, respectively (equivalent to an overall averaged rate of 0.32° C. to 0.25° C./min). The temperature profiles for freezing and thawing in bottles is shown in FIG. 27.

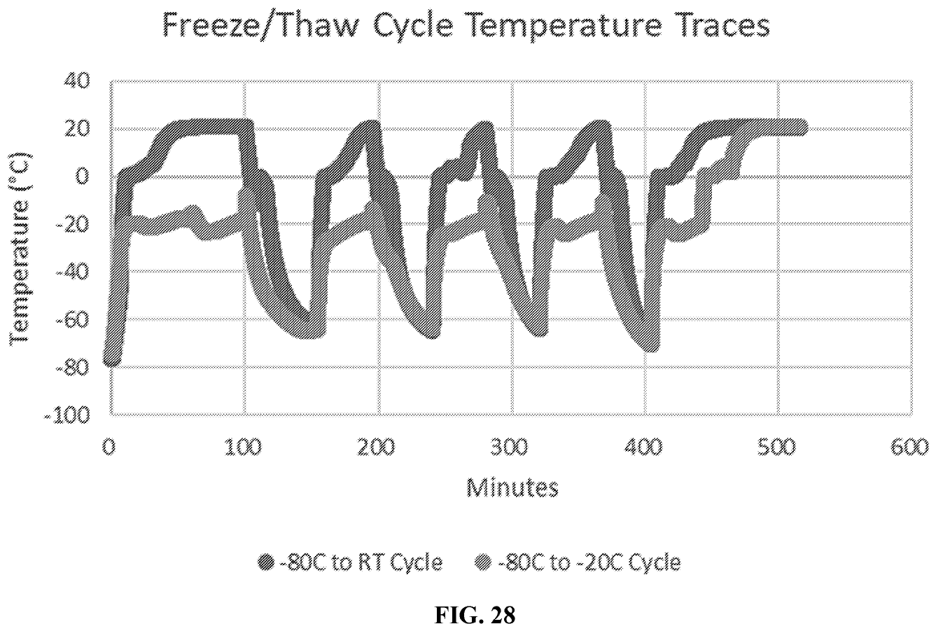

In a separate prior study, freeze/thaw temperature profiles of 0.6 mL water fill into 2 mL Nalgene cryovials was explored. Temperature cycling occurred between a −80° C. freezer and either a −20° C. freezer or benchtop (room temperature). Data is shown in FIG. 28. On average, freezing from room temperature to −60° C. took about 40 minutes while freezing from −20° C. to −60° C. took about 30 minutes. This corresponds to rates of about 2.0° C./min for both studies. Thawing from −60° C. to −20° C. occurred relatively rapid and took about 10 minutes while thawing to room temperature took around 30 minutes. This corresponds to rates of around 4.5° C./min for the −20° C. study and 2.4° C./min for the room temperature study.

In this example, the impact of multiple cycles of freeze/thaw at slow and fast freezing and thawing rates was evaluated. The impact of freeze/thaw rates of about 0.13° C./min (11 hour freeze or thaw) and 1.5° C./min (1 hour freeze or thaw) on the product quality of AAV9.CB7.hCLN2 was assessed. This was performed to further characterize the potential for variability in real-life excursions of temperature on the quality of AAV9. The slow rate was selected to be slower than expected for BDS slow freezing and thawing. For the fast rate, the maximum achievable rate that of about 1° C./min (about 10× faster than the slow rate) was studied as representative of fast thawing and freezing. Multiple cycles were applied to stress the samples beyond what might occur in the clinic. Samples were analyzed by in vitro relative potency, size-exclusion chromatography purity, free DNA levels (using a new SYBR Gold dye-based assay that was found to be sensitive to freeze-thaw stress), and size distribution by dynamic light scattering.

### II. Summary of Freeze-Thaw Studies Results

Overall, the data show that there is minimal impact to the quality attributes of AAV9.CB7.hCLN2 in the intrathecal formulation for five freeze/thaw cycles with rates ranging from as slow as 0.12° C./min (or over about 11 hours) to as fast as 1° C./min (or over about 1 hour).

The freeze/thaw rates were selected to bracket the expected rates that could occur in the clinic for bottles of BDS or vials of DP. Multiple cycles were applied to stress the samples beyond what might occur in the clinic.

An overall summary of the freeze-thaw studies results is provided in Table 4.

TABLE 4
Summary of Findings

| | AAV9.CB7.HCLN2 in formulation | | | |
|---|---|---|---|---|
| | | | | In vitro |
| Freeze and | Free DNA | SE-HPLC² | DLS Cumulants | Relative |
| Thaw Rates³ | (%)¹ | Pre-peak (%) | Diameter (nm) | Potency (%) |
| Control | 1.1 | 0.6 | 27.5 | 93 |
| 5 × FF/FT | 1.7 | 1.2 | 27.3 | 87 |
| 5 × FF/ST | 1.6 | 0.8 | 27.4 | 90 |
| 5 × SF/FT | 1.4 | 0.8 | 27.1 | 97 |
| 5 × SF/ST | 1.1 | 0.8 | 27.1 | 94 |

¹Percent free DNA is based on the measured level compared to the total calculated from GC/mL (OD 260 nm). See results section for more details on the ng/μL levels and comparison to total results after capsid disruption by heat.
²SEC results calculated based on the 260 nm wavelength channel.
³The actual product temperature ‘fast’ rate was about an hour for freezing and 1.5 hours for thawing. The ‘slow’ rate was about 11 hours for both freezing and thawing.

### III. Materials

Vials: CZ 2 mL vials, 13 mm, 19550057 (West, Daikyo)

### Stoppers: 13 mm Serum NovaPure RP S2-F451 4432/50 Gray (West)

AAV9.CB7.hCLN2: Formulated at 3×10¹³ GC/mL in ‘modified Elliott's B intrathecal formulation’ (8.77 g/L sodium chloride, 0.244 g/L magnesium chloride, 0.0278 g/L sodium phosphate monobasic monohydrate, 0.114 g/L sodium phosphate dibasic anhydrous, 0.224 g/L potassium chloride, 0.206 g/L calcium chloride, 0.793 g/L dextrose, 0.001% poloxamer 188, pH 7.26) and vialed at 0.5 mL in CZ vials (Table 5).

TABLE 5
AAV9.CB7.hCLN2 formulation

| | | | | | | Vendor | | Molecular |
|---|---|---|---|---|---|---|---|---|
| | | Quality | Concentration | Concentration | | and Part | Chemical | Weight |
| Ingredient | Function | Standard | (mg/mL) | (mM or %) | | Number | Formula | (g/mol) |
| AAV9.CB7.hCLN2 | API | Internal | Varies based | — | | — | — | — |
| BDS | | | on dose level | | | | | |
| Sodium | Buffering | USP, | 8.77 | 150 | mM | Avantor, | NaCl | 58.440 |
| Chloride | Agent | Ph. Eur, | | | | 3627 | | |
| | Stabilizer | BP, JPE | | | | | | |
| Magnesium | To maintain | USP, BP, | 0.244 | 1.2 | mM | Avantor, | MgCl₂•6H₂O | 203.300 |
| Chloride, 6- | isotonicity | Ph. Eur | | | | 2449 | | |
| Hydrate | | | | | | | | |
| Sodium | | USP, BP | 0.0278 | 0.20 | mM | Avantor, | NaH2PO4•H2O | 137.990 |
| Phosphate | | | | | | 3802 | | |
| Monobasic | | | | | | | | |
| Monohydrate | | | | | | | | |
| Sodium | | USP, | 0.114 | 0.80 | mM | Avantor, | Na₂HPO₄ | 141.960 |
| Phosphate | | Ph. Eur, | | | | 3804 | | |
| Dibasic | | JPE | | | | | | |
| Anhydrous | | | | | | | | |
| Potassium | | USP, BP, | 0.224 | 3.0 | mM | Avantor, | KCl | 74.5513 |
| Chloride | | Ph. Eur, | | | | 3045 | | |
| | | JPE | | | | | | |
| Calcium | | USP, | 0.206 | 1.4 | mM | Avantor, | CaCl₂•2H2O | 147.010 |
| Chloride | | Ph. Eur | | | | 1335 or | | |
| Dihydrate | | USP, | | | | Avantor, | | |
| | | Ph. Eur | | | | 1336 | | |
| Dextrose, | | USP, | 0.793 | 4.4 | mM | Avantor, | C₆H₁₂O₆ | 180.160 |
| Anhydrous | | Ph. Eur. | | | | 1920 | | |
| | | BP, JPE | | | | | | |

| | | | | | | | |
|---|---|---|---|---|---|---|---|
| Poloxamer 188 | Surfactant | NF, | 0.010 | 0.001% | BASF, | HO(C₃H₆O)ₐ | 7680-9510 |
| | | Ph. Eur, | | | 50424596 | (C₂H₄O)_{b}(C₃H₆O)ₐH | |
| | | JPE | | | | | |
| Water | Aqueous | WFI | Approximately | Approximately | Varies | H₂O | 18.0153 |
| | Vehicle | | 994 mg/mL | 55M | | | |

### IV. Equipment

Genesis 25EL Lyophilizer (SP Scientific) Asset Tag 0941 (FFF) with temperature probe thermocouples.

Cytation 5 plate reader (BioTek, Winooski, Vt.), Asset Tag 0867 (FFF instrument)

Cary 60 UV-Visible Spectrophotometer (Agilent, Santa Clara, Calif.), Asset Tag 0999 (FFF)

Thermal mixer/Heat block (Thermo Scientific), S/N: 01014318110768

Waters Acquity Arc Equipment ID 0447 (C3PO) and Sepax SRT SEC-1000 Peek column (PN 215950P-4630, SN: 8A11982, LN: BT090, 5 μm 1000 A, 4.6×300 mm)

TA instruments differential scanning calorimeter, DSC250/RCS90, Serial number DSC2A-00980, Asset Tag 0866.

### V. Methods

A. Controlled Freeze-Thaw Cycles in the Lyophilizer

Controlled freeze/thaw cycles were run in the lyophilizer according to Table 6. Vials were well-spaced on the shelves and 4 vials of buffer were thermocoupled.

TABLE 6
Controlled Freeze and Thaw Rate Settings

| Condition^{a, b} | Freeze Rate | Thaw Rate |
|---|---|---|
| Frozen Control | NA | NA |
| 5 × Fast Freeze/Fast | 25° C. to −60° C. at | −60° C. to 25° C. at |
| Thaw (FF/FT) | 1.5° C./min (1 hour) | 1.5° C./min (1 hour) |
| 5 × Fast Freeze/Slow | 25° C. to −60° C. at | −60° C. to 25° C. at |
| Thaw (FF/ST) | 1.5° C./min^{c} (1 hour) | 0.13° C./min (11 hours) |
| 5 × Slow Freeze/Fast | 25° C. to −60° C. at | −60° C. to 25° C. at |
| Thaw (SF/FT) | 0.13° C./min (11.3 hours) | 1.5° C./min (1 hour) |
| 5 × Slow Freeze/Slow | 25° C. to −60° C. at | −60° C. to 25° C. at |
| Thaw (SF/ST) | 0.13° C./min (11 hours) | 0.13° C./min (11 hours) |

^{a}Shelves were programmed to hold at −60° C. and 25° C. for at least 1 hour between freeze and thaw cycles (there was a longer frozen hold was for some runs for laboratory scheduling purposes).
^{b}All samples and the frozen control were subjected to an uncontrolled freeze to −80° C. in the freezer and a thaw on the bench at room temperature at the end of the study before analysis.
^{c}The first cycle of the FF/ST was run from 25° C. to −55° C. in an attempt to reduce the load on the condenser. The subsequent 4 cycles were set to −60° C.

B. In-vitro Relative Potency

IVRP of AAV9.CB7.hCLN2 was performed. To relate the ddPCR GC titer to functional gene expression, an in vitro bioassay was performed by transducing HEK293 cells and assaying for tripeptidyl peptidase I (TPP1) enzyme activity. HEK293 cells were plated onto three 96-well tissue culture plates overnight. The cells were then pre-infected with wild-type human adenovirus serotype 5 virus followed by transduction with three independently prepared serial dilutions of AAV9.CB7.hCLN2 reference standard and test article, with each preparation plated onto separate plates at different positions. On the second day following transduction, the cells were lysed, treated with low pH to activate the TPP1 enzyme, and assayed for TPP1 enzyme activity using a peptide substrate that yields increased fluorescence signal upon cleavage by TPP1. The fluorescence or RFU was plotted versus log dilution, and the relative potency of each test article was calculated relative to the reference standard on the same plate fitted with a four-parameter logistic regression model after passing the parallelism similarity test, using the formula: EC50 reference±EC50 test article. The potency of the test article was reported as a percentage of the reference standard potency, calculated from the weighted average of the three plates.

C. Free-DNA Analysis

Free DNA was determined by fluorescence of SYBR® Gold nucleic acid gel stain (‘SYBR Gold dye’) that is bound to DNA. The fluorescence was measured using a microplate reader and quantitated with a DNA standard. The results in ng/μL were reported.

Two approaches were used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1E6 is a unit conversion factor:

Total DNA(ng/μL)estimated=1E6×GC/mL(OD)×M(g/mol)/6.02E23

In the second approach the sample was heated to 85° C. for 20 min with 0.05% poloxamer 188 and the actual DNA measured in the heated sample by the SYBR Gold dye assay was used as the total. This therefore has the assumption that all the DNA was recovered and quantitated. The determination of total DNA by the SYBR gold dye (relative to the UV reading) was found to be lower for the AAV9.CB7.hCLN2 (60%) and slightly higher for the Construct II dPBS formulation (131%), and higher still for the Construct II modified dPBS with sucrose formulation (152%). This variation in the conversion of ng/μL to percentage of free DNA was captured as a range in the reported results. For trending, either the raw ng/μL can be used or the percentage determined by a consistent method can be used.

D. Size-Exclusion HPLC Analysis

SEC was performed using a Sepax SRT SEC-1000 Peek column (PN 215950P-4630, SN: 8A11982, LN: BT090, 5 μm 1000 A, 4.6×300 mm) on Waters Acquity Arc Equipment ID 0447 (C3PO), with a 25 mm pathlength flowcell. The mobile phase was (20 mM sodium phosphate, 300 mM NaCl, 0.005% Pluronic F-68, pH 6.5-VA 15 April 19), with a flow rate of 0.35 mL/minute for 20 minutes, with the column at ambient temperature. Data collection was performed with 2 point/second sampling rate and 1.2 nm resolution with 25 point mean smoothing at 214, 260, and 280 nm. The ideal target load was 1.5E11 GC. The AAV9.CB7.hCLN2 were injected with 5 μL.

E. Dynamic Light Scattering

Dynamic light scattering (DLS) was performed on a Wyatt DynaProIII using Corning 3540 384 well plates with a 30 μL sample volume. Ten acquisitions each for 10 s were collected per replicate and there were three replicate measurements per sample. The solvent was set to ‘PBS’ for the AAV9.CB7.hCLN2. Results not meeting data quality criteria (baseline, SOS, noise, fit) were ‘marked’ and excluded from the analysis. The low delay time cutoff was changed from 1.4 μs to 10 μs for the modified dPBS with sucrose samples to eliminate the impact of the sucrose excipient peak at about 1 nm on causing artifactually low cumulants analysis diameter results.

F. Differential Scanning Calorimetry

Low temperature Differential Scanning calorimetry (low-temp DSC) was run using a TA Instruments DSC250. About 20 μL of sample was loaded into a Tzero pan and crimped with a Tzero Hermetic lid. Samples were equilibrated at 25° C. for 2 min, then cooled at 5° C./min to −60° C., equilibrated for 2 min, then heated at 5° C./min to 25° C. Heat flow data was collected in conventional mode.

### VI. Results

A. Freeze-Thaw Study Temperature Profiles

The product temperature did not match the shelf exactly due to heat transfer limitations and phase transitions of the buffer during freezing and melting. The average rates determined using duration between when the product was near 25° C. and −60° C. for a representative portion of the cycle to calculate the overall average rates are summarized in Table 7.

There was a characteristic upward spike in temperature where the product was slightly warmer than the shelf temperature at approximately −10° C. as energy is released during freezing. Similarly, the product was at a slightly lower temperature relative to the shelf during melting of ice at around 0° C. The sections below show the probe temperature data. In addition, rates for the shelf and probes (i.e. 5 point slope of temperature and time) are shown for the FT/FT and the SF/SF as representative of the actual rates achieved for slow and fast rates.

The fast freeze average rate was limited to about 1° C./min and the fast thaw average rate was limited to about 0.8 to 1° C./min.

The actual product temperature ‘fast’ rate was about an hour for freezing and 1.5 hours for thawing. The ‘slow’ rate was about 0.12° C./min taking about 11 hours for both freezing and thawing.

TABLE 7
Actual Product Temperature and Rates during Freeze and Thaw Cycles

| Condition^{1, 2} | Freeze Rate | Thaw Rate |
|---|---|---|
| 5 × Fast Freeze/Fast | Target: 25° C. to −60° C. at | Target: −60° C. to 25° C. at |
| Thaw (FF/FT) | 1.5° C./min (1 hour) | 1.5° C./min (1 hour) |
| | Actual: 20° C. to −3° C. at | Actual: −53° C. to 20° C. at |
| | 1° C./min (~1 hour) | 1° C./min (~1.5 hour) |
| 5 × Fast Freeze/Slow | Target: 25° C. to −60° C. at | Target: −60° C. to 25° C. at |
| Thaw (FF/ST) | 1.5° C./min(1 hour) | 0.13° C./min (11 hours) |
| | Actual: 23° C. to −55° C. at | Actual: −55° C. to 23° C. at |
| | 1.2° C./min (~1 hour) | 0.12° C./min (~11 hours) |
| 5 × Slow Freeze/Fast | Target: 25° C. to −60° C. at | Target:−60° C. to 25° C. at |
| Thaw (SF/FT) | 0.13° C./min (11.3 hours) | 1.5° C./min (1 hour) |
| | Actual: 23° C. to −55° C. at | Actual: −55° C. to 23° C. at |
| | 0.12° C./min (~11 hours) | 0.8° C./min (~1.5 hours) |
| 5 × Slow Freeze/Slow | Target: 25° C. to −60° C. at | Target: −60° C. to 25° C. at |
| Thaw (SF/ST) | 0.13° C./min (11 hours) | 0.13° C./min (11 hours) |
| | Actual: 24° C. to −56° C. at | Actual: −56° C. to 24° C. at |
| | 0.12° C./min (~11 hours) | 0.12° C./min (~11 hours) |

B. Fast Freeze/Fast Thaw (FF/FT)

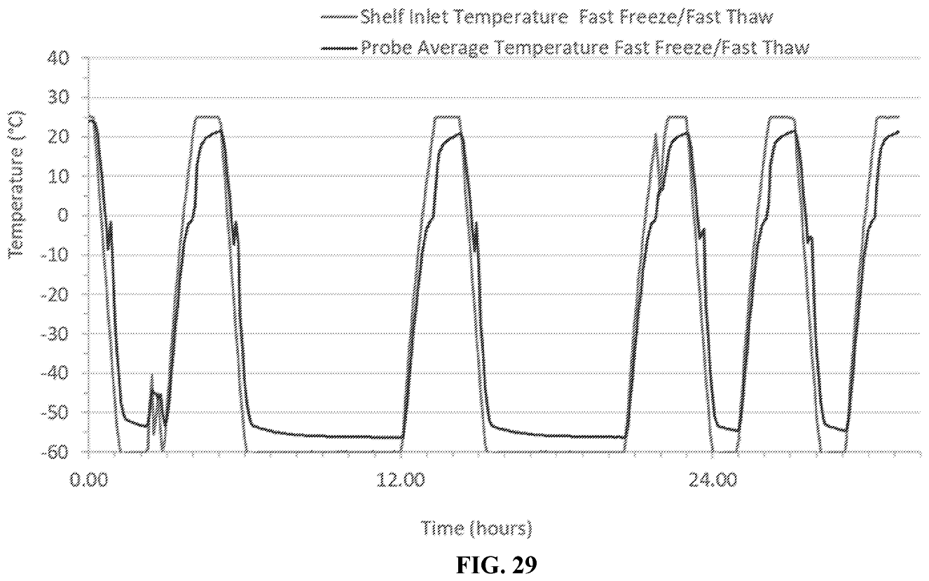

FIG. 29 shows the shelf and probe temperature profile for the FF/FT. There was a longer frozen hold was for some cycles for laboratory scheduling purposes.

The fast freeze average rate was limited to about 1° C./min and the fast thaw average rate was limited to about 0.8 to 1° C./min.

The temperature spike during the frozen portion of the first cycle appears to be an instrument spike. The spike near room temperature on the third cycle was due to a manual reset of the system to continue the cycles and associated temporary (for a few minutes) decrease in shelf temperature setting to a default closer to 10° C.

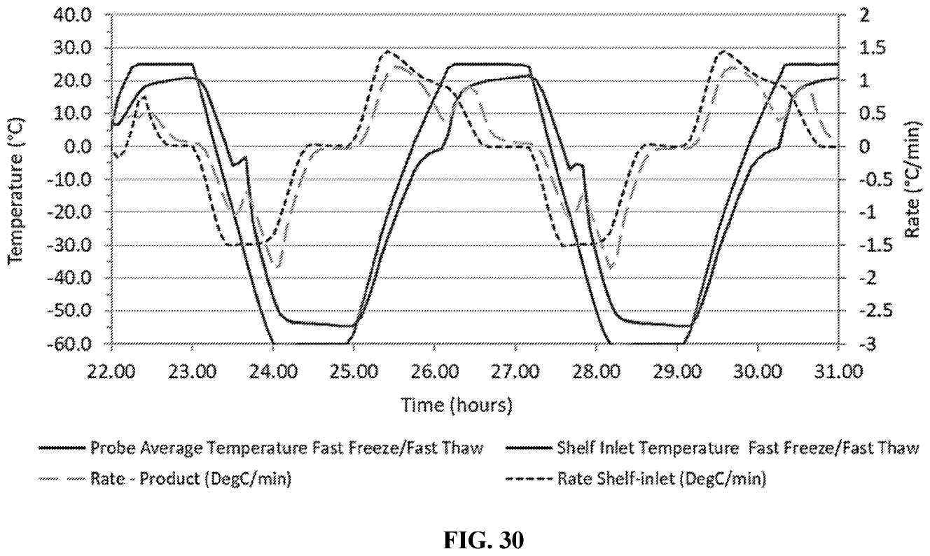

FIG. 30 shows a zoom in of both the shelf and product temperatures and their rates (averaged over 25 min). The actual rates show that the average product and shelf temperatures were impacted by the very rapid freezing and melting and associated limitations of heat transfer during these processes at the fast rates programmed. The melting of the product extracted heat from the shelf and resulted in a reduction in the shelf temperature rate during the melting temperature range.

C. Fast Freeze/Slow Thaw (FF/ST)

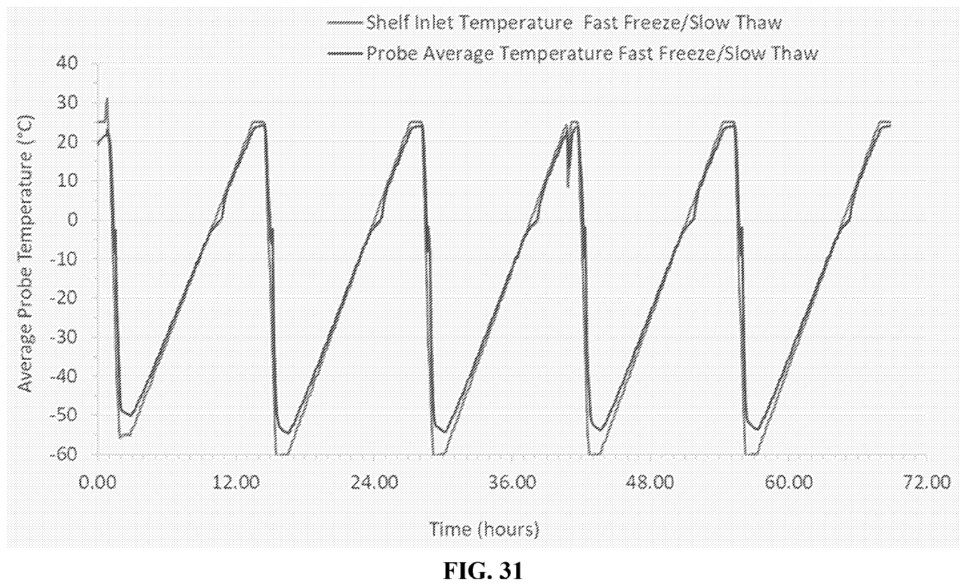

FIG. 31 shows the shelf and probe temperature profile for the FF/ST. The first cycles of the FF/ST were run from 25° C. to −55° C. and back to 25° C. in an attempt to reduce the load on the condenser. The subsequent 4 cycles were set to −60° C. The time to freeze and thaw were not updated which increased the target rates to 1.6° C./min (from 1.5° C./min) and to 0.13° C./min (from 0.125° C./min). This difference is negligible at less than 10% from the original target rates. The spike near room temperature on the third cycle was due to a manual reset of the system to continue the cycles and associated temporary (for a few minutes) decrease in shelf temperature setting to a default closer to 10° C.

D. Slow Freeze/Fast Thaw (SF/FT)

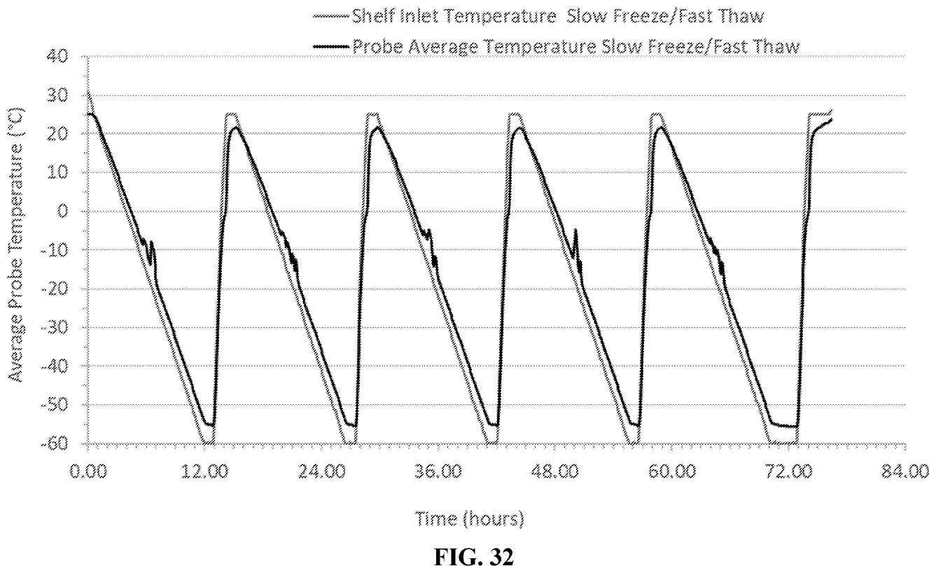

FIG. 32 shows the shelf and probe temperature profile for the SF/FT. There was a longer frozen hold was for the last cycle for laboratory scheduling purposes.

E. Slow Freeze/Slow Thaw (SF/ST)

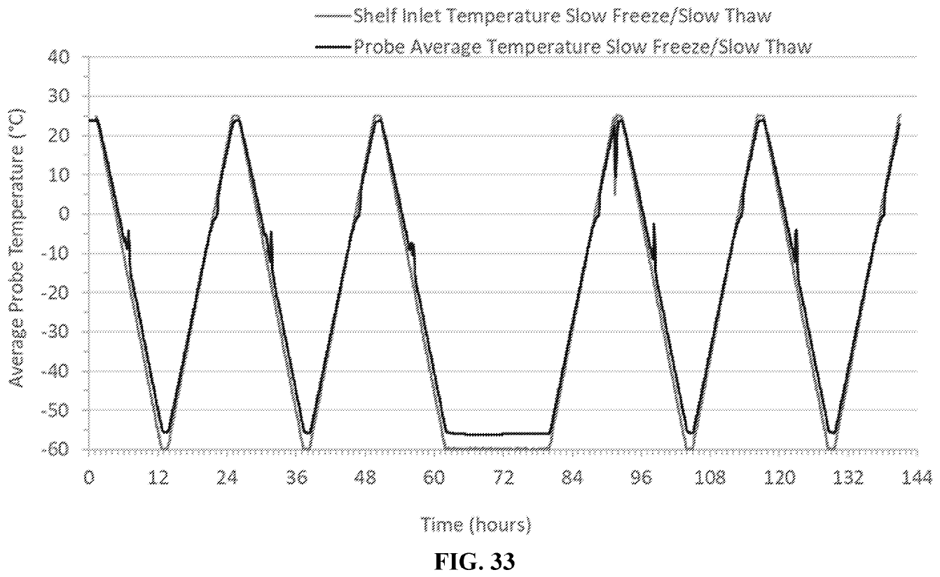

FIG. 33 shows the shelf and probe temperature profile for the FF/FT. There was a longer frozen hold for the third cycles for laboratory scheduling purposes. The spike near room temperature on the third cycle was due to a manual reset of the system to continue the cycles and associated temporary (for a few minutes) decrease in shelf temperature setting to a default closer to 10° C.

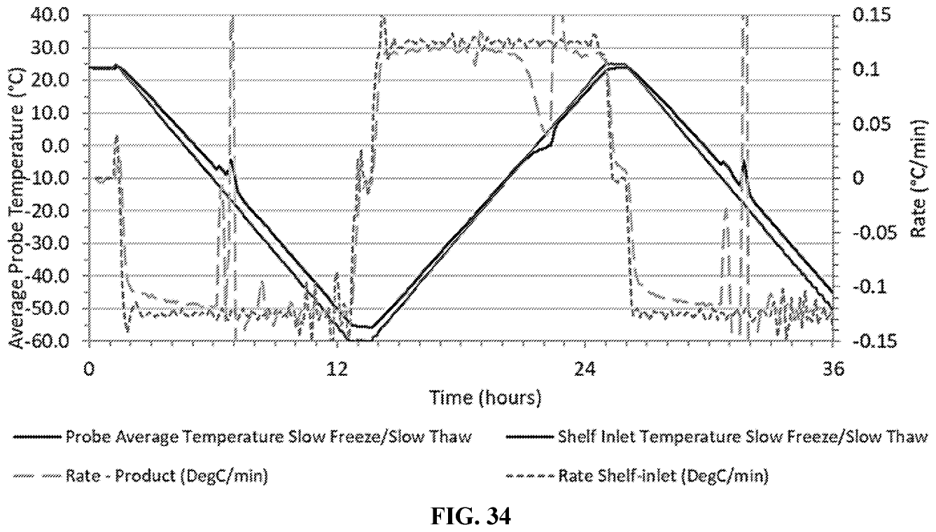

FIG. 34 shows a zoom in of both the shelf and product temperatures and their rates (averaged over 25 min). The rates show that the average product temperatures were impacted by the freezing and melting processes but that the shelf temperature rates remained stable at these slower rates (as compared with the FT/FT).

### VII. In-vitro Relative Potency

The in-vitro relative potency (IVRP) results were similar to the control and within method variability for all permutations of fast and slow rates of freezing and thawing for AAV9.CB7.HCLN2 in the intrathecal formulation (Table 8).

TABLE 8
IVRP Results

| | In-vitro Relative Potency (%) |
|---|---|
| Freeze and | AAV9.CB7.hCLN2 in intrathecal |
| Thaw Rates | formulation |
| Control | 93 |
| 5 × FF/FT | 87 |
| 5 × FF/ST | 90 |
| 5 × SF/FT | 97 |
| 5 × SF/ST | 94 |

### VIII. Free-DNA Results by SYBR Dye Binding and SEC

An overall result summary for free DNA is provided in Table 9. A range is provided for free DNA by SYBR Gold binding which represents the percentage based on either the GC/mL(OD) value for 100% or the heat-stressed result for 100% basis.

TABLE 9
Free DNA by SYBR Gold and Pre-Peak by SEC Results

AAV9.CB7.hCLN2 in intrathecal formulation

| Freeze and | | SE-HPLC^{b} |
|---|---|---|
| Thaw Rates | Free DNA^{a} | Pre-peak (%) |
| Frozen | 1.08 ng/μL | 0.6% |
| Control | 1.1% to 1.9% | Relative 1.0 |
| | Relative 1.0 | |
| 5 × FF/FT | 1.66 ng/μL | 1.2% |
| | 1.7% to 2.9% | Relative 2.1 |
| | Relative 1.5 | |
| 5 × FF/ST | 1.59 ng/μL | 0.8% |
| | 1.6% to 2.7% | Relative 1.3 |
| | Relative 1.5 | |
| 5 × SF/FT | 1.35 ng/μL | 0.8% |
| | 1.4% to 2.3% | Relative 1.5 |
| | Relative 1.3 | |
| 5 × SF/ST | 1.06 ng/μL | 0.8% |
| | 1.1% to 1.8% | Relative 1.4 |
| | Relative 1.0 | |

^{a}Percent free DNA range is calculated as the percentage of the total detected in an 85° C. 20 min heat-stressed sample and the percentage calculated from GC/mL (OD 260 nm). ‘Relative’ is the ratio of the free DNA in the freeze-thaw samples compared to the frozen controls using the GC/mL (OD) values.
^{b}SEC results calculated based on the 260 nm wavelength channel.

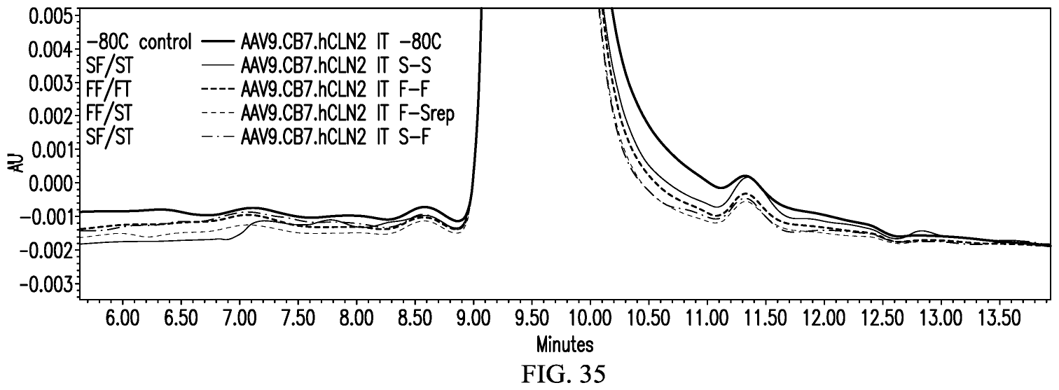

A zoomed-in view of SEC result profiles are shown in FIG. 35. The 260 nm UV channel was used to determine percent pre-peak which represents free DNA (earlier peaks) and some protein (closed pre-peak). A spectral analysis of the peaks and their elution positions indicate that the pre-peaks were predominantly free DNA. Peaks after the main peak are related to excipients.

### IX. Dynamic Light Scattering Results

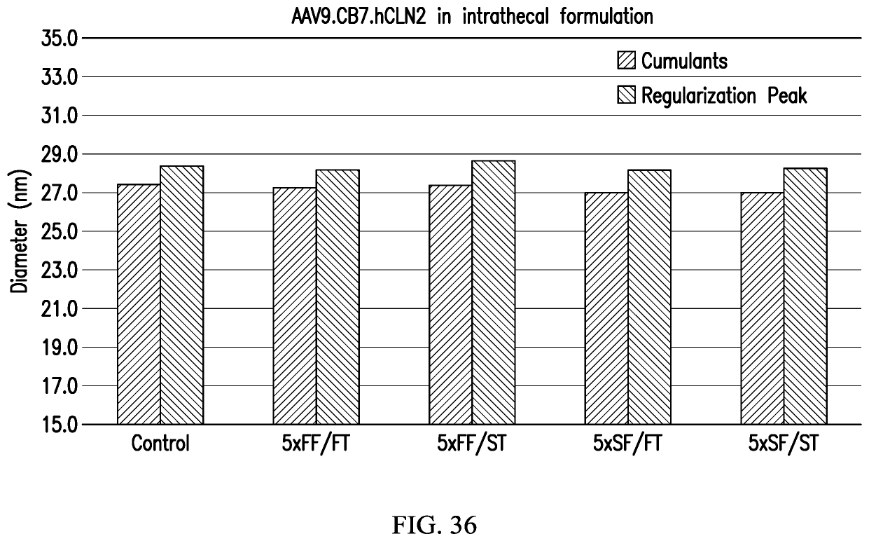

DLS results are shown in FIG. 36. The diameter results for cumulants fitting and the main peak by regularization fitting are shown in Table 10.

There was no change in the size distribution within method variability for AAV9.CB7.hCLN2 after any freeze-thaw condition. The cumulants diameters ranged from 27.1 to 27.5 nm (control=27.5) and the regularization fit results ranged from 28.2 to 28.7 nm (control=28.4). The range in the cumulants data was 0.4 nm and the range in the regularization data was 0.5 nm. The standard deviation of the mean diameter for replicate measurements was about 0.2 for cumulants fitting and up to 0.8 nm for regularization fitting.

TABLE 10
DLS Diameter Results

AAV9.CB7.HCLN2 in
intrathecal formulation

| Freeze and | | Regularization |
|---|---|---|
| Thaw Rates | Cumulants | Peak |
| Control | 27.5 | 28.4 |
| 5 × FF/FT | 27.3 | 28.2 |
| 5 × FF/ST | 27.4 | 28.7 |
| 5 × SF/FT | 27.1 | 28.2 |
| 5 × SF/ST | 27.1 | 28.3 |
| Range in data (nm) | 0.4 | 0.5 |

### X. Thermal Properties of Formulations

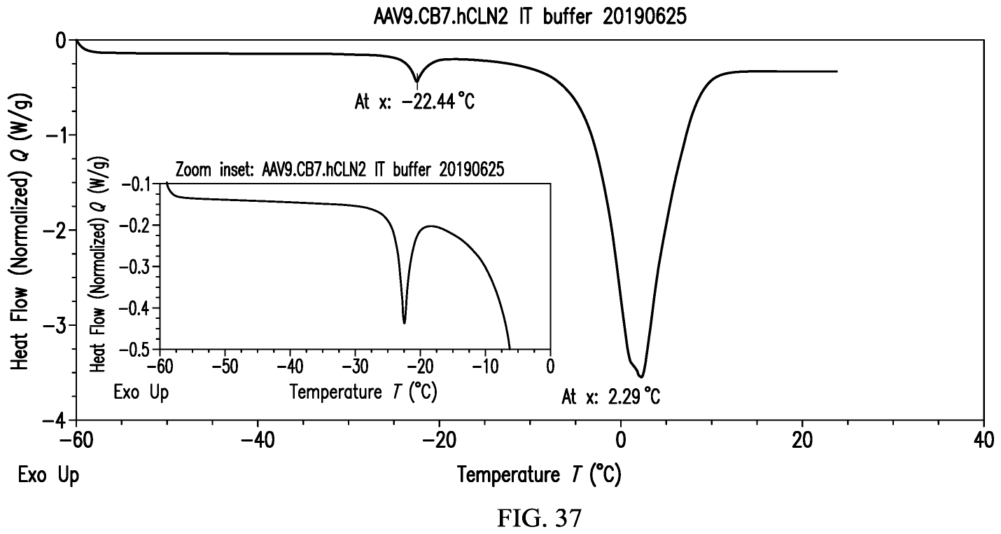

The low temperature DSC thermogram for the modified Elliott's B formulation buffer shown in FIG. 37 has a eutectic melt with a peak at −22.4° C. followed by a large endothermic peak due to melting of ice. No other transitions were observed. The eutectic melt is consistent with a sodium chloride and water eutectic.

### XI. Conclusions

These results of this study demonstrated that up to five freeze/thaw cycles with either slow (0.12° C./min or over about 11 hours) and/or fast (1° C./min or over about 1 hour) rates were acceptable allowable excursions for AAV9.CB7.hCLN2 in the intrathecal formulation.

The freeze/thaw rates were selected to bracket the expected rates that could occur in the clinic for bottles of BDS or vials of DP. Multiple cycles were applied to stress the samples beyond what might occur in the clinic to support multiple excursions.

The potency results were similar to the control and within method variability for all permutations of fast and slow rates of freezing and thawing for AAV9.CB7.hCLN2 in the intrathecal formulation.

There was no change in the size distribution within method variability for AAV9.CB7.hCLN2 intrathecal formulation.

Freeze-thaw stress was shown to disrupt a small number of capsids of AAV9.CB7.hCLN2 (AAV9) resulting in release of small amounts of free DNA when formulated without a cryoprotective excipient.

A very small increase from 1.1 to up to 1.7% free DNA was detected after multiple freeze/thaw for the for AAV9.CB7.hCLN2 in the modified Elliott's B intrathecal formulation. There was little differentiation in results for different rates of fast and slow rates of freezing and thawing. The impact of fast or slow rates were similar.

### Example 4: Studies of Pharmaceutical Compositions Comprising rAAV

This example provides methods that may be used to evaluate the formulations of the pharmaceutical compositions comprising rAAV provided herein, particularly, the methods to compare the formulations to a reference composition, for example the cerebrospinal fluid (CSF).

The formulation buffer (Table 5) closely matched the electrolyte composition, glucose content, and osmolality of cerebrospinal fluid (CSF).

The formulation buffer was different from the composition of CSF in at least that (1) the formulation does not contain bicarbonate buffer; and (2) the formulation contains sodium phosphate monobasic monohydrate for pH buffering. The level of sodium chloride was increased to maintain the overall solution electrolyte content and osmolality. Surprisingly, the removal of the bicarbonate improved the robustness in maintaining the formulation pH on storage. The formulation also included an addition of 0.001% (w/v) poloxamer 188 at least to mitigate the adsorption of capsid particles on the container and other surfaces.

The formulation buffer was different from certain formulations that had been used for delivery to the CSF in approved drugs. For example, the formulations of Spinraza® (nusinersen sodium; an intrathecally administered drug) and Brineura® (cerliponase alfa; the co-packaged electrolytes solution is an intraventricularly administered drug) do not contain dextrose. Surprisingly, the use of dextrose in the AAV9.CB7.hCLN2 formulation may improve compatibility with the CSF. Surprisingly, dextrose in the AAV9.CB7.hCLN2 formulation may inhibit crystallization of salts during freezing and thawing and therefore result in improved stability of the product during shipping and storage logistics.

The comparability of formulation buffer to the CSF is summarized in Table

TABLE 11
Comparison of Electrolyte Composition (pH and Non-electrolytic
Constituents of Formulation Buffer and CSF)

| | Na⁺ | K⁺ | Ca⁺⁺ | Mg⁺⁺ | HCO₃ | Cl⁻ | | Phosphate | Glucose |
|---|---|---|---|---|---|---|---|---|---|
| Solution | mEq/L | mEq/L | mEq/L | mEq/L | mEq/L | mEq/L | pH | mEq/dL | Mg/dL |
| CSF | 117-137 | 2.3-4.6 | 2.2 | 2.2 | 22.9 | 113-127 | 7.31 | 1.2-2.1 | 45-80 |
| Formulation | 151 | 3.0 | 2.8 | 2.4 | 0.0 | 156 | 6.0-7.5 | 1.8 | 79 |
| Buffer ¹ | | | | | | | | | |

¹ Formulation buffer is comparable to cerebrospinal fluid in pH, electrolyte composition, glucose and osmolarity.

The stability of AAV9.CB7.hCLN2 in formulation buffer was confirmed for five freeze/thaw cycles (room temperature ˜23° C. to ≤−60° C.). Permutations of slow (over about 11 hours or 0.13° C./min) and fast (over about 1 hour or 0.8 and 1° C./min) controlled rates were performed to assess robustness to different rates of freezing and thawing. No significant change was observed in free DNA levels, size-exclusion high performance liquid chromatography (SE-HPLC) pre-peak, dynamic light scattering (DLS) diameter (only a single main peak was detected for all samples), or in vitro potency after all permutations of five freeze/thaw cycles between 2-8° C. and ≤−60° C., as shown in Table 12.

TABLE 12
Freeze/Thaw Study Results for Pharmaceutical
Composition Comprising AAV9.CB7.hCLN2

| Freeze/Thaw | Free DNA | SE-HPLC² | DLS | In vitro Relative |
|---|---|---|---|---|
| Condition | (%)¹ | Pre-peak (%) | Diameter(nm) | Potency (%) |
| Control | 1.1 | 0.6 | 27.5 | 93 |
| 5 × fast freeze/fast thaw | 1.7 | 1.2 | 27.3 | 87 |
| 5 × fast freeze/slow thaw | 1.6 | 0.8 | 27.4 | 90 |
| 5 × slow freeze/fast thaw | 1.4 | 0.8 | 27.1 | 97 |
| 5 × slow freeze/slow thaw | 1.1 | 0.8 | 27.1 | 94 |

¹Percent free DNA is based on the measured value using SYBR Gold ® dye fluorescence compared to the total calculated from the UV absorbance at 260 and 280 mu.
²The SE-HPLC pre-peak contains both free DNA and aggregates.

### Example 5: Manufacturing

Briefly, the human embryonic kidney (HEK) 293 suspension cell line is used to produce the AAV9.CB7.hCLN2 vector and is a permanent line transformed by sheared human Adenovirus type 5 DNA (Graham et al., 1977). HEK293 cells from the master cell bank (MCB) are transfected with 3 plasmids to produce the packaged vector genome: a plasmid containing the CLN2 expression cassette (cis plasmid) and 2 helper plasmids encoding AAV9 capsid proteins and replication elements (trans plasmid and Adenovirus Helper plasmid).

The cis plasmid contains the hCLN2 expression cassette and encodes the rAAV vector genome. The hCLN2 expression cassette is flanked by AAV2 inverted terminal repeats (ITRs) and expression is driven by a CB7 promoter, a hybrid between a cytomegalovirus (CMV) immediate-early enhancer and the chicken β-actin promoter. Transcription from this promoter is enhanced by the presence of the chicken β-actin intron (CI). The polyadenylation signal for the expression cassette is from the rabbit β-globin (rBG) gene.

Trans Plasmid: The packaging construct, pAAV29KanRGXRep2. This plasmid encodes the 4 wild-type AAV2 rep proteins and the 3 wild-type AAV VP capsid proteins from AAV9. A novel AAV sequence was obtained from the liver tissue DNA of a rhesus monkey. A PCR fragment of the AAV9 cap gene amplified from liver DNA was inserted as a replacement for the AAV2 cap gene in plasmid p5E18. The plasmid backbone is from pBluescript KS, with the ampicillin resistance gene replaced by the kanamycin resistance gene in the manufacture of pAAV29KanRGXRep2. Due to an altered AAV2 DNA sequence that was discovered in replacing the ampicillin resistance gene, a segment of DNA was removed by restriction enzyme digest and replaced with a newly synthesised DNA fragment so that the rep gene encoded the correct AAV2 rep amino acid sequence. The identity of the plasmid pAAV29KanRGXRep2 capsid gene was confirmed by DNA plasmid sequencing performed by Genewiz.

Adenovirus Helper Plasmid: Plasmid pAdDeltaF6 contains regions of the Adenovirus (Ad) genome that are important for AAV replication, namely E2A, E4 and viral-associated (VA) RNA. pAdDeltaF6 does not encode any additional Ad replication or structural genes and does not contain cis elements, such as the Ad ITRs, that are necessary for replication; therefore, no infectious Ad is expected to be generated. Adenoviral E1 essential gene functions are supplied by the HEK293 cells in which the rAAV vectors are produced.

Each of the cis, trans and helper plasmids described above contains a kanamycin-resistance cassette, so β-lactam antibiotics are not used in their production. This eliminates the concern that patients will show reactivity to β-lactam antibiotics (Mignon et al., 2015).

The manufacturing process for bulk drug substance is summarized in the flow diagrams presented in FIG. 38 and FIG. 39. The bulk drug substance is manufactured by polyethylenimine (PEI)-mediated transient transfection of the HEK293 cells with the three plasmids described above. The vector is produced in the HEK293 cells and released from the cells into culture supernatant. The recombinant AAV is then purified and concentrated using several orthogonal methods.

The cell culture and harvest manufacturing process comprises 4 main manufacturing steps: 1) vial thaw, cell seeding and expansion in a shake flasks and disposable bag bioreactor, 2) transient transfection, 3) post-transfection media feeds, and 4) crude cell supernatant harvest. The purification process comprises 6 main manufacturing steps: 1) vector harvest clarification by filtration, 2) concentration and diafiltration by tangential flow filtration (TFF), 3) purification by affinity chromatography, 4) purification by ion exchange chromatography, 5) concentration and buffer exchange by hollow fibre TFF into formulation buffer, and 6) final filtration, filling, and freezing.

Suspension: Alternatively, an in-house, suspension master cell bank (MCB) is derived from an established, characterized, adherent HEK293 MCB by adaptation of cells into suspension culture using serum-free and animal component-free culture medium. The suspension HEK293 MCB was tested extensively for microbial and viral contamination. Additional testing was conducted to confirm the absence of a range of specific pathogens of human, simian, bovine, and porcine origin. The human origin of the HEK293 MCB was demonstrated by polymerase chain reaction (PCR) and standard tandem repeat (STR) profiling. The MCB is used to produce GMP vector.

Seed propagation method increased volume to achieve a target cell density. The time range for the conduct of the suspension main bioreactor process was 5 to 8 days. During that time, the pH, dissolved oxygen and temperature levels were controlled. The downstream suspension process follows closely the sequence of operations that is described for the alternative cell culture process.

Transient Transfection: Following a few days of cell growth to desired cell densities in the production bioreactor, cells are transfected with the 3 production plasmids using an optimized polyethylenimine (PEI)-based transfection method with pAAV.CB7.CI.CLN2.RBG.KanR vector genome plasmid, pAdDeltaF6, pAAV29KanRGXRep2 AAV plasmid and PEI. After mixing, the solution is allowed to sit at room temperature for several minutes and then added to serum-free media to quench the reaction and then added to the bioreactor. The cells are incubated at 37° C. for a few days.

Vector Harvest: The cell culture is supplemented with MgCl₂ to a final concentration of a few mM (co-factor for Benzonase) and Benzonase nuclease is added. The harvest material is mixed and incubated at 37° C. for a few hours to provide sufficient time for enzymatic digestion of residual cellular and plasmid DNA present in the harvest as a result of the transfection procedure. This step is performed to minimize the amount of residual DNA in the final vector drug product. After the incubation period, NaCl is added to a final concentration of 500 mM to end digestion and aid in the recovery of the product during filtration and downstream tangential flow filtration (TFF).

Vector Clarification: Cells and cellular debris are removed from the Benzonase-treated harvest material using a filter. Bioburden reduction filtration ensures that at the end of the filter train, any bioburden potentially introduced during the upstream production process will be reduced before downstream purification.

Vector Purification Process

The purification process comprises 4 main manufacturing steps that are described in detail below: concentration and buffer exchange by TFF, affinity chromatography, ion exchange chromatography, and concentration and buffer exchange by TFF.

Concentration and Buffer Exchange by Tangential Flow Filtration: Volume reduction (10-fold) of the clarified product is achieved by TFF using a custom sterile, closed bioprocessing tubing, bag and membrane set. The principle of TFF is to flow a solution under pressure parallel to a membrane of suitable porosity (100 kDa or tighter). The pressure differential drives molecules of smaller size through the membrane and effectively into the waste stream while retaining molecules larger than the membrane pores. By recirculating the solution, the parallel flow sweeps the membrane surface preventing membrane pore fouling. By choosing an appropriate membrane pore size and surface area, a liquid sample may be rapidly reduced in volume while retaining and concentrating the desired molecule. Diafiltration in TFF applications involves addition of a fresh buffer to the recirculating sample at the same rate that liquid is passing through the membrane and to the waste stream. With increasing volumes of diafiltration, increasing amounts of the small molecules are removed from the recirculating sample. This results in a modest purification of the clarified product, but also achieves buffer exchange compatible with the subsequent affinity column chromatography step.

Affinity Chromatography: The diafiltered product is subsequently applied to an affinity matrix that efficiently captures the AAV9 serotype. Under these ionic conditions, a significant percentage of residual cellular DNA and proteins flow through the column, while AAV9 particles are efficiently captured. Following application, the column is washed using two buffers to remove additional feed impurities followed by a low pH step elution. The eluate is immediately neutralized by the addition of a neutralization buffer

Ion Exchange Chromatography: To achieve further reduction of in-process impurities including empty AAV particles, an ion exchange chromatography step is employed. For this step, the affinity elution pool is diluted multiple-fold to reduce ionic strength to enable binding to a to the ion exchange matrix. Following a low-salt wash, vector product is eluted using a multiple-column volume (CV) NaCl linear salt gradient. This shallow salt gradient separates capsid particles without a vector genome (empty particles) from particles containing vector genome (full particles) and results in a preparation enriched in full capsids.

Concentration and Buffer Exchange by Hollow Fiber Tangential Flow Filtration: The pooled ion exchange intermediate is concentrated and buffer exchanged by using TFF. During the step, the product is brought to a determined target concentration. Following this concentration step, buffer exchange is achieved by diafiltration with multiple volumes of formulation buffer. Samples are removed for BDS testing after 0.2 μm filtration. The process time for the concentration and buffer exchange step is less than 1 day.

Filling and Storage: Following filtration and sampling, the BDS is filled into bottles and stored frozen at ≤−60° C. in a quarantine location until release for FDP processing.

TABLE 13

| Proposed Formulation for | Proposed formulation for |
|---|---|
| AAV9.CB7.hCLN2 Excipient | AAV9.CB7.hCLN2 (mM) |
| Sodium Chloride | 150 |
| Magnesium Chloride | 1.2 |
| Potassium Chloride | 3 |
| Calcium Chloride | 1.4 |
| Sodium Phosphate | 1 |
| Dextrose | 4.4 |
| Poloxamer 188 | 0.001% |

Thaw and Pooling: Frozen aliquots of BDS are thawed at room temperature. Multiple BDS batches may be pooled and mixed via swirling. The thawed BDS may be held overnight at 2-8° C.

Optional Concentration by TFF or Dilution: The BDS may be concentrated using hollow fiber TFF, or diluted with formulation buffer, to achieve the desired concentration (in GC/mL). The optional concentration step is performed using TFF identical to the hollow fiber TFF concentration step in the BDS process.

Sterile Filtration: A bioburden sample of the DP intermediate is taken immediately prior to filtration. The DP is 0.22 μm filtered using a pre-sterilized assembly. The 0.22 μm filter is flushed with formulation buffer, then drained. The DP intermediate is then filtered, and the filter, which is pre-use integrity tested by the filter manufacturer, is post-use integrity tested prior to filling the DP into vials. If the filter fails post-use integrity testing, the filtered intermediate may be re-filtered using a different filter.

Filling, Storage, and Transportation: Filtered DP is filled into CZ vials using a peristaltic pump. The initial vials will be filled at a volume optimized for testing (for example 1 mL in a 10 mL vial). These will be used for sterility, endotoxin, and other release or stability testing. The fill volume will be increased for the next set of vials (for example 5 mL in a 10 mL vial), which will be used in the clinic. The final set of vials will again be filled at a lower volume (for example 1 mL in a 10 mL vial), and used for sterility, endotoxin, and other release or stability testing. Weight checks are performed at pre-defined intervals. Vials are capped and crimped, then 100% visually inspected, labelled, packaged in cartons, and frozen at ≤−60° C.

The drug product proposed configuration is a 1 mL frozen solution of AAV9.CB7.hCLN2 vector in formulation buffer contained in a 2 mL vial. The proposed formulation buffer is 150 mM sodium chloride, 1.2 mM magnesium chloride, 3 mM potassium chloride, 1.4 mM calcium chloride, 1 mM sodium phosphate, 4.4 mM dextrose, and 0.001% poloxamer 188, pH 7.3. The proposed quantitative composition of drug product is provided in the Table 14 below.

TABLE 14
Proposed Quantitative Composition of AAV9.CB7.hCLN2
Solution for Injection, 1 mL/Vial

| Material | Function | Grade | Amount (per vial) | |
|---|---|---|---|---|
| AAV9.CB7.hCNLN2 | Active | GMP | >1 × 10¹³ | GC/mL |
| | Substance | | | |
| Sodium Chloride | Stabiliser | USP/Ph. Eur./ | 8.76 | mg/mL |
| | | JP/BP/FCC | | |
| Magnesium Chloride | Stabiliser | USP/Ph. Eur./ | 0.11 | mg/mL |
| | | JP/BP/FCC | | |
| Potassium Chloride | Stabiliser | USP/Ph. Eur./ | 0.22 | mg/mL |
| | | JP/BP/FCC | | |
| Calcium Chloride | Stabiliser | USP/Ph. Eur./ | 0.16 | mg/mL |
| | | JP/BP/FCC | | |
| Sodium Phosphate | Stabiliser | USP/Ph. Eur./ | 0.16 | mg/mL |
| | | JP/BP/FCC | | |
| Dextrose | Stabiliser | USP/Ph. Eur./ | 0.79 | mg/mL |
| | | JP/BP/FCC | | |
| Poloxamer 188 | Surfactant | GMP | 0.001 | mL |
| Water for Injection | Solvent | USP/Ph. Eur. | q.s. to 1.0 | mL/vial |

Fill and finish is performed under aseptic conditions in accordance with regulatory guidelines for sterile products. The drug product is a clear, colorless solution that has been developed as a single-use, sterile solution for injection. The drug product is to be administered via IC injection.

For shipping to clinical sites, the FDP vials previously packaged into cartons are placed into a pre-qualified cardboard shipping box with a temperature logger. The box is filled with dry ice to maintain the shipping temperature of ≤−60° C. Upon receipt of the shipment, the temperature logger is read to confirm no temperature excursion during shipping.

All published documents cited in this specification are incorporated herein by reference, as are U.S. Provisional Patent Application No. 62/652,006, filed Apr. 3, 2018, U.S. Provisional Patent Application No. 62/599,816, filed Dec. 18, 2017 and U.S. Provisional Patent Application No. 62/504,817, filed May 11, 2017. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### Sequence Listing Free Text

The following information is provided for sequences containing free text under numeric identifier <223> or <213>.

| SEQ ID NO: | |
|---|---|
| (containing | |
| free text) | Free text under <223> or <213> |
| 3 | <223> constructed sequence |
| 5 | <223> constructed sequence |
| 6 | <213> adeno-associated virus 9 |
| 7 | <213> adeno-associated virus 9 |
| 8 | <223> constructed sequence |

What is claimed is:

1. A method of treating CLN2 Batten disease in a subject comprising administering to a subject in need thereof a recombinant adeno-associated virus (rAAV) via a first route and a second route, wherein said first route and said second route are into the central nervous system (CNS), wherein said first route is into the brain region and said second route is into the spinal cord region, and
wherein said recombinant adeno-associated virus (rAAV) comprises an AAV capsid and a vector genome packaged therein, and wherein said vector genome comprising:
(a) an AAV 5′ inverted terminal repeat (ITR) sequence;
(b) a promoter;
(c) a CLN2 coding sequence encoding a human TPP1; and
(d) an AAV 3′ ITR.

2. The method of claim 1, wherein said first route is intracerebroventricular (ICV) or intracisternal (IC).

3. The method according to any one of claims 1 to 2, wherein said second route is intrathecal-lumbar (IT-L).

4. The method according to any one of claims 1 to 3, wherein said method further comprises administering to said subject said rAAV via a third route, wherein said third route is selected from the group consisting of intracerebroventricular (ICV), intracisternal (IC), intrathecal-lumbar, intracranial, intravenous, intravascular, intraarterial, intramuscular, intraocular, subcutaneous, and intradermal.

5. A method of treating CLN2 Batten disease in a subject comprising administering to a subject in need thereof a recombinant adeno-associated virus (rAAV) via a first route and a second route, wherein said first route is into the central nervous system (CNS), wherein said second route delivers the rAAV to the liver, and
wherein said recombinant adeno-associated virus (rAAV) comprises an AAV capsid and a vector genome packaged therein, and wherein said vector genome comprising:
(a) an AAV 5′ inverted terminal repeat (ITR) sequence;
(b) a promoter;
(c) a CLN2 coding sequence encoding a human TPP1; and
(d) an AAV 3′ ITR.

6. The method of claim 5, wherein said first route is intrathecal-lumbar (IT-L), intracerebroventricular (ICV) or intracisternal (IC).

7. The method according to any one of claims 5 to 6, wherein said second route is selected from the group consisting of intravenous, intravascular, intraarterial, intramuscular, intraocular, subcutaneous, and intradermal.

8. The method according to claim 7, wherein said second route is intravenous.

9. The method according to any one of claims 1 to 8, wherein said method comprises administering said rAAV via said first route simultaneously with administering said rAAV via said second route.

10. The method according to any one of claims 1 to 8, wherein said method comprises administering said rAAV via said first route prior to administering said rAAV via said second route.

11. The method according to any one of claims 1 to 8, wherein said method comprises administering said rAAV via said first route after administering said rAAV via said second route.

12. The method according to any one of claims 10 to 11, wherein the interval between administration said rAAV via said first route and administering said rAAV via said second route is about 0.5 hour, about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

13. The method according to any one of claims 1 to 12, wherein said method results in a TPP1 activity in the spinal cord of said subject that is at least 2%, 3%, 5%, 6%, 7%, 8%, 9%, 10%, 12%, 15%, 17%, 20%, 25%, 30%, 35%, 40%, 45%, or 50% higher than a reference TPP1 activity in the spinal cord of a second subject, and wherein the reference TPP1 activity in the spinal cord is measured when said second subject does not receive the treatment using said method, and wherein said second subject is the same or different from said subject.

14. The method according to any one of claims 1 to 13, wherein said method results in a hepatic TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference hepatic TPP1 activity in a second subject, and wherein the reference hepatic TPP1 activity is measured when said second subject does not receive the treatment using said method, and wherein said second subject is the same or different from said subject.

15. The method according to any one of claims 1 to 14, wherein said method results in a serum TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference serum TPP1 activity in a second subject, and wherein the reference serum TPP1 activity is measured when said second subject does not receive the treatment using said method, and wherein said second subject is the same or different from said subject.

16. The method according to any one of claims 1 to 15, wherein said method results in a microglial activity in the cortex of said subject that is at least 2%, 3%, 5%, 6%, 7%, 8%, 9%, 10%, 12%, 15%, 17%, 20%, 25%, 30%, 35%, 40%, 45%, or 50% lower than a reference microglial activity in the cortex in a second subject, and wherein the reference microglial activity in the cortex is measured when said second subject does not receive the treatment using said method, and wherein said second subject is the same or different from said subject.

17. The method according to any one of claims 1 to 16, wherein said method results in a TPP1 activity in the brain of said subject that is at least 2%, 3%, 5%, 6%, 7%, 8%, 9%, 10%, 12%, 15%, 17%, 20%, 25%, 30%, 35%, 40%, 45%, or 50% higher than a reference TPP1 activity in the brain of a second subject, wherein the reference TPP1 activity in the brain is measured when said second subject does not receive the treatment using said method, and wherein said second subject is the same or different from said subject.

18. The method according to any one of claims 1 to 17, wherein said rAAV is administered in a therapeutically effective amount.

19. The method according to any one of claims 1 to 18, wherein said subject is human.

20. The method according to any one of claims 1 to 19, wherein the coding sequence of (c) is a codon optimized human CLN2, which is at least 70% identical to the native human coding sequence of SEQ ID NO: 2.

21. The method according to any one of claims 1 to 20, wherein the coding sequence of (c) is SEQ ID NO: 3.

22. The method according to any one of claims 1 to 21, wherein the rAAV capsid is an AAV9 or a variant thereof.

23. The method according to any one of claims 1 to 22, wherein the promoter is a chicken beta actin (CBA) promoter.

24. The method according to any one of claims 1 to 23, wherein the promoter is a hybrid promoter comprising a CBA promoter sequence and cytomegalovirus enhancer elements.

25. The method according to any of claims 1 to 24, wherein the AAV 5′ ITR and/or AAV3′ ITR is from AAV2.

26. The method according to any of claims 1 to 25, wherein the vector genome further comprises a polyA.

27. The method according to claim 26, wherein the polyA is a synthetic polyA or from bovine growth hormone (bGH), human growth hormone (hGH), SV40, rabbit β-globin (RGB), or modified RGB (mRGB).

28. The method according to any of claims 1 to 27, wherein the vector genome further comprises an intron.

29. The method according to claim 28, wherein the intron is from CBA, human beta globin, IVS2, SV40, bGH, alpha-globulin, beta-globulin, collagen, ovalbumin, or p53.

30. The method according to any of claims 1 to 29, wherein the vector genome further comprises an enhancer.

31. The method according to claim 30, wherein the enhancer is a CMV enhancer, an RSV enhancer, an APB enhancer, ABPS enhancer, an alpha mic/bik enhancer, TTR enhancer, en34, ApoE.

32. The method according to any of claims 1 to 31, wherein the vector genome is about 3 kilobases to about 5.5 kilobases in size.

33. The method according to any of claims 1 to 32, wherein the vector genome is about 4 kilobases in size.

34. The method according to any of claims 1 to 33, wherein the rAAV is manufactured using a method comprising growing in suspension culture a suspension cell line that is capable of producing the rAAV.

35. The method according to claim 34, wherein said suspension cell line is HEK293 suspension cell line.

36. A pharmaceutical composition comprising:
(a) a recombinant adeno-associated virus (rAAV),
(b) sodium chloride,
(c) magnesium chloride,
(d) potassium chloride,
(e) dextrose,
(f) poloxamer 188,
(g) sodium phosphate monobasic, and
(h) sodium phosphate dibasic,
wherein said recombinant adeno-associated virus (rAAV) comprises an AAV capsid and a vector genome packaged therein, and wherein said vector genome comprising: (i) an AAV 5′ inverted terminal repeat (ITR) sequence; (ii) a promoter; (iii) a CLN2 coding sequence encoding a human TPP1; and (iv) an AAV 3′ ITR.

37. The pharmaceutical composition according to claim 36 further comprising calcium chloride.

38. The pharmaceutical composition according to claim 37, wherein said sodium chloride, said magnesium chloride, said potassium chloride, said dextorse, said poloxamer 188, said sodium phosphate monobasic, said sodium phosphate dibasic, and said calcium chloride are each in anhydrous, monohydrate, dihydrate, 3-hydrate, 4-hydrate, 5-hydrate, 6-hydrate, 7-hydrate, 8-hydrate, 9-hydrate, or 10-hydrate form.

39. The pharmaceutical composition according to any one of claims 36 to 38 comprising:
(a) said rAAV,
(b) sodium chloride at a concentration of about 8.77 g/L,
(c) magnesium chloride 6-hydrate, at a concentration of about 0.244 g/L,
(d) potassium chloride at a concentration of about 0.224 g/L,
(e) calcium chloride dihydrate at a concentration of about 0.206 g/L,
(f) dextorse anhydrous at a concentration of about 0.793 g/L,
(g) poloxamer 188 at a concentration of about 0.001% (volume/volume),
(h) sodium phosphate monobasic monohydrate at a concentration of about 0.0278 g/L, and
(i) sodium phosphate dibasic anhydrous at a concentration of about 0.114 g/L.

40. The pharmaceutical composition according to any one of claims 36 to 39, wherein the vector genome concentration (VGC) of the pharmaceutical composition is about 1×10¹¹ GC/mL, about 3×10¹¹ GC/mL, about 6×10¹¹ GC/mL, about 1×10¹² GC/mL, about 3×10¹² GC/mL, about 6×10¹² GC/mL, about 1×10¹³ GC/mL, about 2×10¹³ GC/mL, about 3×10¹³ GC/mL, about 4×10¹³ GC/mL, about 5×10¹³ GC/mL, about 6×10¹³ GC/mL, about 7×10¹³ GC/mL, about 8×10¹³ GC/mL, about 9×10¹³ GC/mL, or about 1×10¹⁴ GC/mL, about 3×10¹⁴ GC/mL, about 6×10¹⁴ GC/mL, or about 1×10¹⁵ GC/mL.

41. The pharmaceutical composition according to any one of claims 36 to 40, wherein the pH of the pharmaceutical composition is in a range from about 6.0 to about 9.0.

42. The pharmaceutical composition according to claim 41, wherein the pH of the pharmaceutical composition is about 7.4.

43. The pharmaceutical composition according to any one of claims 36 to 42, wherein said rAAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant rAAV in a reference pharmaceutical composition.

44. The pharmaceutical composition according to claim 43, wherein the stability of said rAAV is determined by
(a) the infectivity of rAAV,
(b) the levels of aggregation of rAAV, or
(c) the levels of free DNA released by the rAAV.

45. The pharmaceutical composition according to any one of claims 36 to 44, wherein the pharmaceutical composition is a liquid composition.

46. The pharmaceutical composition according to any one of claims 36 to 44, wherein the pharmaceutical composition is a frozen composition.

47. The pharmaceutical composition according to any one of claims 36 to 44, wherein the pharmaceutical composition is a lyophilized composition or a reconstituted lyophilized composition.

48. The pharmaceutical composition according to any one of claims 36 to 47, wherein the pharmaceutical composition has a property that is suitable for intracerebroventricular (ICV), intracisternal (IC), intrathecal-lumbar, intracranial, intravenous, intravascular, intraarterial, intramuscular, intraocular, intramuscular, subcutaneous, or intradermal administration.

49. The pharmaceutical composition according to any one of claims 36 to 48, wherein the coding sequence of (iii) is a codon optimized human CLN2, which is at least 70% identical to the native human coding sequence of SEQ ID NO: 2.

50. The pharmaceutical composition according to any one of claims 36 to 49, wherein the coding sequence of (iii) is SEQ ID NO: 3.

51. The pharmaceutical composition according to any one of claims 36 to 50, wherein the rAAV capsid is an AAV9 or a variant thereof.

52. The pharmaceutical composition according to any one of claims 36 to 51, wherein the promoter is a chicken beta actin (CBA) promoter.

53. The pharmaceutical composition according to any one of claims 36 to 52, wherein the promoter is a hybrid promoter comprising a CBA promoter sequence and cytomegalovirus enhancer elements.

54. The pharmaceutical composition according to any one of claims 36 to 53, wherein the AAV 5′ ITR and/or AAV3′ ITR is from AAV2.

55. The pharmaceutical composition according to any one of claims 36 to 54, wherein the vector genome further comprises a polyA.

56. The pharmaceutical composition according to any one of claims 36 to 55, wherein the polyA is a synthetic polyA or from bovine growth hormone (bGH), human growth hormone (hGH), SV40, rabbit β-globin (RGB), or modified RGB (mRGB).

57. The pharmaceutical composition according to any one of claims 36 to 56, wherein the vector genome further comprises an intron.

58. The pharmaceutical composition according to any one of claims 36 to 57, wherein the intron is from CBA, human beta globin, IVS2, SV40, bGH, alpha-globulin, beta-globulin, collagen, ovalbumin, or p53.

59. The pharmaceutical composition according to any one of claims 36 to 58, wherein the vector genome further comprises an enhancer.

60. The pharmaceutical composition according to any one of claims 36 to 59, wherein the enhancer is a CMV enhancer, an RSV enhancer, an APB enhancer, ABPS enhancer, an alpha mic/bik enhancer, TTR enhancer, en34, ApoE.

61. The pharmaceutical composition according to any one of claims 36 to 60, wherein the vector genome is about 3 kilobases to about 5.5 kilobases in size.

62. The pharmaceutical composition according to any one of claims 36 to 61, wherein the vector genome is about 4 kilobases in size.

63. The pharmaceutical composition according to any one of claims 36 to 62, wherein the rAAV is manufactured using a method comprising growing in suspension culture a suspension cell line that is capable of producing the rAAV.

64. A method of treating CLN2 Batten disease in a subject comprising administering to said subject the pharmaceutical composition according to any one of claims 36 to 63.

65. The method according to claim 64, wherein said pharmaceutical composition is administered in a therapeutically effective amount.

66. The method according to any one of claims 64 to 65, wherein said subject is human.

67. A kit comprising one or more containers and instructions for use, wherein the one or more containers comprise the pharmaceutical composition according to any one of claims 36 to 63.


SEQUENCE LISTING (8 sequences)

SEQ ID NO: 1 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 2 - DNA, 1693 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgac

SEQ ID NO: 3 - DNA, 1693 bp, Artificial Sequence
  constructed sequence
atgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgat

SEQ ID NO: 4 - protein, 320 aa, Homo sapiens
MRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 5 - DNA, 6841 bp, Artificial Sequence
  constructed sequence
tcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgatgac

SEQ ID NO: 6 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 2211 bp, adeno-associated virus 9
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 8 - DNA, 4020 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag